(19) United States
(12) Patent Application Publication
(10) Pub. No.: US 2025/0257337 A1
(43) Pub. Date: Aug. 14, 2025

(54) AUGMENTING CAR T CELL ACTIVITY

(71) Applicant: THE TRUSTEES OF THE UNIVERSITY OF PENNSYLVANIA, PHILADELPHIA, PA (US)

(72) Inventors: James L. Riley, Downingtown, PA (US); Meidi Gu, Havertown, PA (US)

(21) Appl. No.: 19/048,503

(22) Filed: Feb. 7, 2025

Related U.S. Application Data
(60) Provisional application No. 63/551,910, filed on Feb. 9, 2024.

(51) Int. Cl. C12N 9/14; A61K 40/11; A61K 40/31; A61K 40/42; A61P 35/00; C07K 16/28; C07K 16/30; C12N 5/0783

(52) U.S. Cl. CPC C12N 9/14; A61K 40/11; A61K 40/31; A61K 40/4211; A61K 40/4253; A61K 40/4255; A61P 35/00; C07K 16/2803; C07K 16/30; C12N 5/0636; C12Y 306/05002; C07K 2319/00; C12N 2510/00

(57) ABSTRACT

The present invention relates to fusion proteins comprising a chimeric antigen receptor linked to a second polypeptide by a linker domain, wherein the second polypeptide prolongs the surface expression of the CAR, and wherein the linker is a cleavable linker. Also included are nucleic acids encoding the same, methods of treatments, and other methods or uses thereof.

### CROSS-REFERENCE TO RELATED APPLICATION

The present application is entitled to priority under 35 U.S.C. § 119 (e) to U.S. Provisional Patent Application No. 63/551,910, filed Feb. 9, 2024, which is hereby incorporated herein by reference in its entirety.

### SEQUENCE LISTING

The present application contains a Sequence Listing which has been submitted in XML format via Patent Center and is hereby incorporated by reference in its entirety. Said XML copy, created on Feb. 6, 2025, is named “046483-7445US1(03802) Sequence Listing ST_26.xml” and is 17.9 kilobytes in size.

### BACKGROUND

Engineered T cells and cellular therapies using them have revolutionized the treatment and management of many cancers. Among engineered T cell therapies, Chimeric Antigen Receptors, or CARs, represent one of the most successful strategies. While CAR T cells can be very effective in some patients, recent clinical experiences with CAR-expressing T cells have demonstrated that durable expression of the CAR constructs significantly correlates with durable patient responses and better treatment outcomes. Here, downregulation of CAR construct expression reduces the cytotoxic function of the engineered T cell, and thus negative affects treatment efficacy. CAR construct surface expression is regulated, in part, by ligand-induced down-regulation. Thus, there is a need in the art for strategies that help maintain surface expression of CAR constructs, thereby optimizing T cell-based cellular therapies. The current invention addresses this need.

### SUMMARY OF THE INVENTION

As described herein, the present disclosure relates to fusion proteins comprising a chimeric antigen receptor (CAR) linked to a second polypeptide by a linker domain, wherein the second polypeptide prolongs the surface expression of the CAR, and wherein the linker is a cleavable linker. Also included are nucleic acids encoding the same, methods of treatments comprising the same, and other methods or uses thereof.

As such, in one aspect, the present invention provides a fusion protein comprising a chimeric antigen receptor linked to a second polypeptide by a linker domain, wherein the CAR comprises an antigen binding domain, a transmembrane domain, and an intracellular domain, and wherein the second polypeptide prolongs the surface expression of the CAR, and wherein the linker is a cleavable linker.

In certain embodiment, the second polypeptide is a Rab5 protein.

In certain embodiments, the second polypeptide is a Rab11 protein.

In certain embodiments, the linker is a self-cleaving peptide.

In certain embodiments, the self-cleaving peptide is a 2A peptide.

In certain embodiments, the CAR has a binding specificity for a cancer-related antigen.

In certain embodiments, the cancer-related antigen is CD19.

In certain embodiments, the cancer-related antigen is mesothelin.

In certain embodiments, the cancer-related antigen is selected from a group consisting of: TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In certain embodiments, the intracellular domain comprises a signaling domain selected from the group consisting of a 4-1BB signaling domain, a CD28 signaling domain, and a KIR signaling domain.

In certain embodiments, the 4-1BB signaling domain comprises the amino acid sequence set forth in SEQ ID NO: 8.

In certain embodiments, the intracellular domain comprises a CD3zeta signaling domain.

In certain embodiments, the CD3zeta domain comprises an amino acid sequence set forth in SEQ ID NO: 9.

In certain embodiments the KIR signaling domain is a KIR2DS2 domain and comprises the amino acid sequence set forth in SEQ ID NO: 41.

In certain embodiments, the fusion protein of the above embodiments or any other aspect or embodiment disclosed herein further comprises a CD8 leader sequence.

In certain embodiments, the CD8 leader sequence comprises the amino acid sequence set forth in SEQ ID NO: 4.

In certain embodiments, the transmembrane domain is a CD8 transmembrane domain.

In certain embodiments, the CD8 transmembrane domain comprises the amino acid sequence set forth in SEQ ID NO: 5.

In certain embodiments, the fusion protein comprises the amino acid sequence set forth in SEQ ID NO: 1.

In certain embodiments, the fusion protein comprises the amino acid sequence set forth in SEQ ID NO: 2.

In certain embodiments, the second polypeptide comprises the amino acid sequence set forth in SEQ ID NO: 3.

In certain embodiments, the cleavable linker comprises the amino acid sequence set forth in SEQ ID NO: 10.

In certain embodiments, the CAR comprises the amino acid sequence set forth in SEQ ID NOs: 1-2 or 43-85

In another aspect, the present invention provides a nucleic acid vector encoding the modified fusion protein of any of the embodiments or aspects disclosed herein.

In certain embodiments, the vector is an expression vector.

In another aspect, the present invention provides a modified immune cell or precursor thereof, comprising the fusion protein of claim 1 or the nucleic acid vector of any of the embodiments or aspects disclosed herein.

In certain embodiments, the immune cell is selected from the group consisting of a T cell, a macrophage, and a NK cell.

In certain embodiments, the immune cell is a T cell.

In certain embodiments, the T cell is selected from the group consisting of a CD4 T cell, a CD8 T cell, and a NK T cell.

In certain embodiments, the T cell is a CD8 T cell.

In another aspect, the present invention provides a method of treating cancer in a subject in need thereof, comprising administering to the subject an effective amount of a modified immune cell comprising a fusion protein comprising a chimeric antigen receptor linked to a second polypeptide by a linker domain; wherein the CAR comprises an antigen binding domain, a transmembrane domain, and an intracellular domain, and wherein the second polypeptide prolongs the surface expression of the CAR, and wherein the linker is a cleavable linker.

In certain embodiments, the second polypeptide is a Rab5 protein.

In certain embodiments, the second polypeptide is a Rab11 protein.

In certain embodiments, the linker is a self-cleaving peptide.

In certain embodiments, the self-cleaving peptide is a 2A peptide.

In certain embodiments, the CAR has a binding specificity for a cancer-related antigen.

In certain embodiments, the cancer-related antigen is CD19.

In certain embodiments, the cancer-related antigen is mesothelin.

In certain embodiments, the cancer-related antigen is selected from a group consisting of: TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In certain embodiments, the intracellular domain comprises a signaling domain selected from the group consisting of a 4-1BB signaling domain, a CD28 signaling domain, and a KIR2DS2 domain.

In certain embodiments, the 4-1BB signaling domain comprises the amino acid sequence set forth in SEQ ID NO: 8.

In certain embodiments, the KIR2DS2 domain comprises the amino acid sequence set forth in SEQ ID NO: 41.

In certain embodiments, the intracellular domain comprises a CD3zeta signaling domain.

In certain embodiments, the CD3zeta domain comprises an amino acid sequence set forth in SEQ ID NO: 9.

In certain embodiments of any of the aspects or embodiments disclosed herein, the method further comprises a CD8 leader sequence.

In certain embodiments, the CD8 leader sequence comprises the amino acid sequence set forth in SEQ ID NO: 4.

In certain embodiments, the transmembrane domain is a CD8 transmembrane domain.

In certain embodiments, the CD8 transmembrane domain comprises the amino acid sequence set forth in SEQ ID NO: 5.

In certain embodiments, the fusion protein comprises the amino acid sequence set forth in SEQ ID NO: 1.

In certain embodiments, the fusion protein comprises the amino acid sequence set forth in SEQ ID NO: 2.

In certain embodiments, the second polypeptide comprises the amino acid sequence set forth in SEQ ID NO: 3.

In certain embodiments, the cleavable linker comprises the amino acid sequence set forth in SEQ ID NO: 10.

In certain embodiments, the CAR comprises an amino acid sequence selected from the group consisting of SEQ ID NOs: 1-2 and 43-85.

In certain embodiments, the cancer is selected from the group consisting of B cell acute lymphoblastic leukemia, chronic lymphocytic leukemia, myeloma, mesothelioma, ovarian cancer, lung cancer, squamous carcinoma, non-pulmonary adenocarcinoma, pancreatic cancer, stomach cancer, and colon cancer.

In certain embodiments, the subject is human.

### BRIEF DESCRIPTION OF THE DRAWINGS

The following detailed description of embodiments of the present invention will be better understood when read in conjunction with the appended drawings. For the purpose of illustrating the present invention, there are shown in the drawings embodiments which are presently preferred. It should be understood, however, that the present invention is not limited to the precise arrangements and instrumentalities of the embodiments shown in the drawings.

FIG. 1 is a map of an exemplary CD19 CAR/Rab5 expressing construct.

FIG. 2 is a map of an exemplary mesothelin CAR/Rab5 expressing construct.

FIGS. 3A-3F illustrate that CAR expression is downregulated after repeated tumor challenge or at low effector to target ratios. FIG. 3A. Schematic representation of coculture with CAR-T and tumor cells. FIG. 3B. Primary human T cells were activated with CD3/28 coated beads transduced with a lentiviral vector that expresses a CD19 CAR linked to 4-1BB and CD3zeta signaling domains. After 9 days of expansion these cells were cultured with K562 cells expressing CD19 as indicated in FIG. 3A. Surface CAR expression level was determined by flow cytometry using APC-labelled CD19 on the indicated days. FIG. 3C. GFP+CD19+K562 cells population was analyzed in the co-cultured described in FIG. 3B. FIG. 3D. Overlay of surface CAR expression profile and tumor cell survive profile summarized from (FIG. 3B) and (FIG. 3C). FIGS. 3E-3F. Human CD19 (FIG. 3E) or mesothelin (FIG. 3F) CAR T cells were generated as described in FIG. 3B. and coculture with GFP+CD19+K562 cells (FIG. 3E) or EM-MESO-GFP+cells (FIG. 3F) as indicated effector to target ratio.

FIGS. 4A-4D illustrate that tumor cells uptake CAR when mixed with CAR T cells. FIG. 4A. Primary human cells were transduced with a lentiviral vector encoding CD19−BBz CAR fused with the mCherry. After 9 days of culture, these cells were mixed CD19+K562 cells expressing GFP, and these cells were cocultured as the indicated time point and CAR location was visualized using confocal microscopy. FIG. 4B. Co-cultures were permeabilized and stained with APC-labeled CD19 protein 24 hr after the 3rd cycle of coculture with null transduced human T cells (gray), CD19−BBz CAR-expression human T cells (black) and CAR expression visualized by flow cytometry. FIG. 4C. Representative TEM images showing CAR is released from T cells cross synapse. Human T cell expressing CD19−BBz-APEX2 CAR (dark) incubate with GFP+CD19+K562 cells (light) for overnight coculture. Scale bar is 1 μm (left) and 500 nm (right). FIG. 4D. Representative TEM images showing CAR is up taken into tumor cells. Human T cell null transducing or expressing CD19−BBz-APEX2 incubate with GFP+CD19+K562 cells. 24 hr after the 3rd cycle of coculture, T cells were removed by CD3+ beads, TEM analysis GFP+CD19+K562 cells containing CD19−BBz-APEX2 CAR (black) in the vesicles. Scale bar is 1 μm.

FIGS. 5A-5E illustrate that engineering CD19−CAR with RAB5 increased its persistence and activity under repeated tumor stimulation. FIG. 5A. Schematic showing CD19−BBz-RAB5 CAR design. FIG. 5B. Primary human T cells were transduced with the indicated vector and cultured for 9 days. Immunoblot analysis of RAB5 protein levels is displayed. FIG. 5C-5E. The experiment described in FIG. 3 was repeated with the addition of CAR T cells that co-expressed RAB5.

FIGS. 6A-6D illustrate that Rab5-armed MSLN-CAR-T improves in vitro tumor control. FIG. 6A. Schematics of the MSLN-BBz-RAB5 CAR design. FIG. 6B. Primary human T cells were transduced with the indicated vector and cultured for 18 days. Immunoblot analysis of RAB5 protein levels is displayed. FIG. 6C-6D. FACS analysis (top) and quantification (bottom) of surface CAR expression level (FIG. 6C) and EM-MESO-GFP+ cells population (FIG. 6D). MSLN-BBz CAR-expression human T cells were cocultured with EM-MESO-GFP+ cells as shown in FIG. 3A at indicated time point.

FIGS. 7A-7C illustrate that Rab5 regulates surface CAR persistence directly. FIG. 7A. Immunoblot analysis of CAR level in CD19−BBz CAR or CD19−BBz-RAB5 CAR-expression human T cells isolated from four different donors (D1-D4). CD19−CAR T cells incubate with GFP+CD19+K562 cells. 48 hr after the 3rd cycle of coculture, T cells were isolated by FACS sorting for immunoblotting. FIG. 7B. Volcano plot of RNA sequencing analysis of differentially expressed genes in CD19−BBz CAR-expressing human T cells relative to CD19−BBz-RAB5 CAR-expressing human T cells. All the samples are the same as shown in FIG. 7A. FIG. 7C. Immunoblot analysis of CD19 induced signaling pathway in CD19−BBz CAR-T and CD19−BBz-RAB5 CAR-T cells.

FIGS. 8A-8C describe a RAB5 mutant vector design strategy. FIG. 8A. The Rab GTPase cycle mechanism. FIG. 8B. Immunoblot analysis of protein level of wild-type or mutant RAB5 in CD19−CAR-T cells. FIG. 8C. RAB5 dominant negative vector design.

FIGS. 9A-9D illustrate that RAB5 enables CAR expression dependent on its GTPase function. FACS analysis (FIGS. 9A, 9C) and quantification (FIGS. 9B, 9D) of GFP+CD19+K562 cells population (FIG. 9A) and surface CAR expression level (FIG. 9C). CD19−BBz CAR, CD19−BBz-RAB5 CAR and CD19−BBz-RAB5-S35N CAR-expression human T cells were coculture with GFP+CD19+K562 cells as shown in FIG. 1.

FIGS. 10A-10B illustrate that Rab5 promotes surface CAR endocytosis. FIG. 10A. Diagram illustrating the different types of endocytosis. CME: clathrin-mediated endocytosis. CIE: clathrin-independent endocytosis. FIG. 10B. FACS analysis of surface CAR expression in the presence of indicated inhibitors. CD19−BBz CAR or CD19−BBz-RAB5 CAR-expression human T cells were coculture with GFP+CD19+K562 cells, 24 hr after the 3rd cycle of coculture, CAR-T cells were treated with inhibitors for additional 24 hr, the surface CAR level was detected by APC-CD19 staining.

FIGS. 11A-11E illustrate that RAB5 maintains CAR surface persistence via promoting CAR endocytosis under repeated tumor stimulation. FIGS. 11A-11B. Representative confocal images showing CAR location after recognizing tumor cells. CD19−BBz (BBz) and CD19−BBz-RAB5 (R5) CAR fused BFP (blue) (FIG. 11A) or mCherry (red) (FIG. 11B), CD19+K562 cells fused GFP (green), coculture as the indicated time point. FIG. 11C. FACS analysis of CAR expression level inside of GFP+CD19+K562 cells. Intracellular staining of CAR with tumor cells 24 hr after the 3rd cycle of coculture with null transduced human T cells (gray), CD19−BBz CAR-expression human T cells (black) or CD19−BBz-RAB5 CAR-expression human T cells (green) separately. FIG. 11D. Representative TEM images showing CAR is up taken into the vehicles of tumor cells. Human T cell null transducing, expressing CD19−BBz-APEX2 or CD19−BBz-APEX2-RAB5 incubate with GFP+CD19+K562 cells. 24 hr after the 3rd cycle of coculture, T cells were deleted by CD3+ beads, TEM analysis GFP+CD19+K562 cells containing APEX2 CAR (black) in the vesicles. Scale bar is 1 um. FIG. 11E. Quantification of APEX2-CAR of FIG. 11D. Each dot represents a single GFP+CD19+K562 cell.

FIGS. 12A-12D illustrate that CAR-T constructs with RAB5 fusion demonstrated improved antitumor efficacy. FIG. 12A. 1M GFP T2A luciferase CD19+K562 cells were implanted in NSG mice. Seven days later, 1M of the indicated CAR-T cells linked to mCherry were adoptive transferred to the tumor bearing mice by i.v. Bioluminescence imaging of the mice was performed on the indicated days. FIGS. 12B-12D. Peripheral blood was obtained 21 days after CAR T cell infusion from surviving NSG mice and the number of total T cells (top) and CAR T cells (bottom) by flow cytometry. Data is summarized in FIGS. 12C-12D with each dot representing the data obtained from a mouse.

### DETAILED DESCRIPTION

### Definitions

In the detailed description, certain specific details are set forth in order to provide a thorough understanding of various embodiments. However, one skilled in the art will understand that the embodiments provided can be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The articles “a” and “an” are used herein to refer to one or to more than one (i.e., to at least one) of the grammatical object of the article. By way of example, “an element” means one element or more than one element. As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules comprising two heavy chain and two light chain polypeptides. Each polypeptide chain contains three complementarity-determining regions (CDRs), which bind to the antigen and defines the antibody's antigen specificity.

As used herein, the term “antibody” and “antibodies” can also include polypeptides or polypeptide complexes derived from full-length antibodies. These polypeptide complexes can be naturally occurring or constructed from single chain antibodies or antibody fragments and retain an antigen-specific binding ability. The antibodies of the present invention can exist in a variety of forms including, for example, polyclonal antibodies, monoclonal antibodies, Fv, Fab and F(ab′)₂, as well as single chain antibodies, scFv, humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). For preparation of suitable antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies of this invention. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding can be the result of two distinct binding moieties that are structurally distinct at the molecular level, including but not limited to distinct non-identical amino acid sequences; or a single binding moiety that is able to specifically bind to two structurally distinct targets with high affinity (e.g., with a KD less than about 1×10⁻⁶). A molecule, peptide, polypeptide, antibody, or antibody fragment referred to as “multi-specific” refers to a molecule that possesses the ability to specifically bind to at least three structurally distinct targets. A “bispecific antibody” including grammatical equivalents refers to a bispecific molecule that preserves at least one fragment of an antibody able to specifically bind a target, for example, a variable region, heavy or light chain, or one or more complementarity determining regions from an antibody molecule. A “multi-specific antibody” including grammatical equivalents refers to a multi-specific molecule that preserves at least one fragment of an antibody able to specifically bind with a target, for example, a variable region, heavy or light chain, or complementarity determining region from an antibody molecule.

A “linker” herein is also referred to as “linker sequence” “spacer” “tethering sequence” or grammatical equivalents thereof. A “linker” as referred herein connects two distinct molecules that by themselves possess target binding, catalytic activity, or are naturally expressed and assembled as separate polypeptides, or comprise separate domains of the same polypeptide. For example, two distinct binding moieties or a heavy-chain/light-chain pair. A number of strategies can be used to covalently link molecules together. Linkers described herein can be utilized to join a light chain variable region and a heavy chain variable region in an scFv molecule; or can be used to tether an scFv or other antigen binding fragment on the N- or C-terminus of an antibody heavy chain; or the N- or C-terminus of a light chain to create a bispecific or multi-specific binding molecule. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide can predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length can be used. Useful linkers include glycine-serine polymers, including for example (GS)n, (GSGGS)n (SEQ ID NO: 11), (GGGGS)n (SEQ ID NO: 12), and (GGGS)n (SEQ ID NO: 13), where n is an integer of at least one, glycine-alanine polymers, alanine-serine polymers, and other flexible linkers. Exemplary, linkers for linking antibody fragments or single chain variable fragments can include AAEPKSS (SEQ ID NO: 14), AAEPKSSDKTHTCPPCP (SEQ ID NO: 15), GGGG (SEQ ID NO: 16), or GGGGDKTHTCPPCP (SEQ ID NO: 17). Alternatively, a variety of non-proteinaceous polymers, including but not limited to polyethylene glycol (PEG), polypropylene glycol, polyoxyalkylenes, or copolymers of polyethylene glycol and polypropylene glycol, can find use as linkers.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3): 657-70, (“Aho” numbering scheme); and Whitelegg N R and Rees A R, “WAM: an improved algorithm for modelling antibodies on the WEB,” Protein Eng. 2000 December; 13(12): 819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR can vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In certain embodiments, the CDRs of the antibodies described herein can be defined by IMGT method.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain can be sufficient to confer antigen-binding specificity. Furthermore, antibodies that bind a particular antigen can be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

An “epitope” refers to the binding determinant of an antibody or fragment described herein minimally necessary for specific binding of the antibody or fragment thereof to a target antigen. When the target antigen is a polypeptide, the epitope will be a continuous or discontinuous epitope. A continuous epitope is formed by one region of the target antigen, while a discontinuous epitope can be formed from two or more separate regions. A discontinuous epitope, for example, can form when a target antigen adopts a tertiary structure that brings two amino acid sequences together and forms a three-dimensional structure bound by the antibody. When the target antigen is a polypeptide, the epitope will generally be a plurality of amino acids linked into a polypeptide chain. A continuous epitope can comprise 5, 6, 7, 8, 9, 10, 11, 12, 13, 14, 15, 16, 17, 18, 19, or 20 contiguous amino acids. While an epitope can comprise a contiguous polymer of amino acids, not every amino acid of the polymer can be contacted by an amino acid residue of the antibody. Such non-contacted amino acids will still comprise part of the epitope as they can be important for the structure and linkage of the contacted amino acids. The skilled artisan can determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

The term “antibody fragment” refers to a polypeptide comprising or derived from a portion of an intact antibody and comprises the antigen-binding determining variable regions of an intact antibody. Examples of antibody fragments include, but are not limited to, Fab, Fab′, F(ab′)₂, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multi-specific antibodies formed from antibody fragments. Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A Fab or Fab fragment contains the constant domain of the light chain and the first constant domain (CH1) of the heavy chain. Fab′ or Fab′ fragments differ from Fab fragments by the addition of a few residues at the carboxyl terminus of the heavy chain CH1 domain including one or more cysteines from the antibody hinge region. Fab′ fragments are produced by cleavage of the disulfide bond at the hinge cysteines of the F(ab′)₂ pepsin digestion product. Additional chemical couplings of antibody fragments are known to those of ordinary skill in the art. Fab and F(ab′)₂ fragments lack the Fragment crystallizable (Fc) region of an intact antibody, clear more rapidly from the circulation of animals, and can have less nonspecific tissue binding than an intact antibody. “Fv” fragment is the minimum fragment of an antibody that contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VHVL dimer. In some cases, the six CDRs confer target binding specificity to the antibody. However, in some cases. even a single variable domain (or half of an Fv comprising only three CDRs specific for a target) can have the ability to recognize and bind target. Single domain antibodies (sdAb)/single-chain fragments are composed of a single VH or VL domain which exhibit sufficient affinity to an antigen. A scFv (Single-chain Fv) refers to antibody binding fragments comprise the VH and VL domains of an antibody, where these domains are present in a single polypeptide chain. Generally, the scFv polypeptide further comprises a polypeptide linker between the VH and VL domains which enables the scFv to form a structure favorable for target binding.

The term “linear antibodies” generally refers to the antibodies comprise a pair of tandem Fd segments (VH-CH1-VH-CH1) which, together with complementary light chain polypeptides, form a pair of antigen binding regions. Linear antibodies can be bispecific or monospecific.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response can involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the present invention in prevention of the occurrence of tumor in the first place.

As used herein, the term “autoimmune disease” is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, cancer, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

As used herein, the term “disease” refers to a state of health of a subject wherein the subject cannot maintain homeostasis, and wherein if the disease is not ameliorated then the subject's health continues to deteriorate. In contrast, the term “disorder” in a subject refers to a state of health in which the subject is able to maintain homeostasis, but in which the subject's state of health is less favorable than it would be in the absence of the disorder. Left untreated, a disorder does not necessarily cause a further decrease in the subject's state of health.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the present invention can be replaced with other amino acid residues from the same side chain family and the altered antibody can be tested for the ability to bind GFRα4 using the functional assays described herein.

“Co-stimulatory ligand”, as the term is used herein, includes a molecule expressed by an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to cell-surface molecules expressed by T cells that specifically bind with co-stimulatory ligands expressed by antigen-presenting cells (APCs), thereby providing a “secondary signal” which, in combination with the “primary signal” delivered through MHC/HLA-antigen interactions with the T Cell Receptor (TCR) results in optimal T cell activation including, but not limited to, cytokine production and proliferation. Co-stimulatory molecules include, but are not limited to CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83.

As used herein, “disease associated with expression of a tumor antigen” includes, but is not limited to, a disease associated with cells which express a tumor antigen including, but not limited to proliferative diseases such as a cancer or malignancy or a precancerous condition such as a myelodysplasia, a myelodysplastic syndrome or a preleukemia; or a noncancer related indication associated with cells, which express a tumor antigen. In some embodiments, a cancer associated with expression of a tumor antigen is a hematological cancer. In some embodiments, a cancer associated with expression of a tumor antigen is a solid cancer. Further diseases associated with expression of a tumor antigen include, but not limited to atypical and/or non-classical cancers, malignancies, precancerous conditions or proliferative diseases associated with expression of a tumor antigen. Non-cancer related indications associated with expression of a tumor antigen include, but are not limited to, autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiment, the tumor antigen-expressing cells produced detectable levels of a tumor antigen protein at one point, and subsequently produced substantially no detectable tumor antigen protein.

The term “dysregulated” when used in the context of the level of expression or activity of PD-1 or it's ligands PD-L1 and PD-L2 refers to the level of expression or activity that is different from the expression level or activity of PD-1 or it's ligands in an otherwise identical healthy animal, organism, tissue, cell or component thereof. The term “dysregulated” also refers to the altered regulation of the level of expression and activity of PD-1, PD-L1, compared to the regulation in an otherwise identical healthy animal, organism, tissue, cell or component thereof.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

“Effective amount” or “therapeutically effective amount” are used interchangeably herein, and refer to an amount of a compound, formulation, material, or composition, as described herein effective to achieve a particular biological result. Such results can include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” and “chimeric” forms of non-human (e.g., mouse) antibodies are immunoglobulins, immunoglobulin chains or fragments thereof (such as Fv, Fab, Fab′, F(ab′)₂ or other antigen-binding subsequences of antibodies) which contain minimal sequences derived from non-human immunoglobulin. For the most part, humanized and chimeric antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized and chimeric antibodies can comprise residues which are found neither in the recipient antibody nor in the imported CDR or framework sequences. These modifications are made to further refine and optimize antibody performance. In general, the humanized and chimeric antibody will comprise substantially all of at least one, and typically two, variable domains, in which all or substantially all of the CDR regions correspond to those of a non-human immunoglobulin and all or substantially all of the FR regions are those of a human immunoglobulin sequence. The chimeric antibody optimally also will comprise at least a portion of an immunoglobulin constant region (Fc).

As used herein, an “instructional material” includes a publication, a recording, a diagram, or any other medium of expression which can be used to communicate the usefulness of the compositions and methods of the present invention. The instructional material of the kit of the present invention can, for example, be affixed to a container which contains the nucleic acid, peptide, and/or composition of the present invention or be shipped together with a container which contains the nucleic acid, peptide, and/or composition. Alternatively, the instructional material can be shipped separately from the container with the intention that the instructional material and the compound be used cooperatively by the recipient.

“Identity” as used herein refers to the percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or can be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA can also include introns to the extent that the nucleotide sequence encoding the protein can in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques. The term “polynucleotide” as used herein is defined as a chain of nucleotides.

Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence can be the core promoter sequence and in other instances, this sequence can also include an enhancer sequence and other regulatory elements which are required for expression of the gene product. The promoter/regulatory sequence can, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell. An example of a “cell surface receptor” is human GFRα4.

“Single chain antibodies” refer to antibodies formed by recombinant DNA techniques in which immunoglobulin heavy and light chain fragments are linked to each other using an engineered span of amino acids to recapitulate the Fv region of an antibody as a single polypeptide. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The term “subject” is intended to include living organisms in which an immune response can be elicited (e.g., mammals). A “subject” or “patient,” as used therein, can be a human or non-human mammal. Non-human mammals include, for example, livestock and pets, such as ovine, bovine, porcine, canine, feline and murine mammals.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell and/or on a tumor cell.

A “stimulatory ligand,” as used herein, means a ligand that when present on an antigen presenting cell (e.g., an aAPC, a dendritic cell, a B-cell, and the like) or a tumor cell, can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a super-agonist anti-CD28 antibody, and a super-agonist anti-CD2 antibody.

Ranges: throughout this present invention, various aspects of the present invention can be presented in a range format. It should be understood that the description in range format is merely for convenience and brevity and should not be construed as an inflexible limitation on the scope of the present invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

In some aspects, the current invention provides chimeric antigen receptor (CAR) constructs which also comprise Rab5 or Rab11 polypeptides. Co-expression of the Rab5 or Rab11 polypeptides help maintain or prolong surface expression of the CAR construct by opposing mechanisms which reduce or remove CAR complexes from the surface of the immune cell or T cell.

By the term “prolongs the surface expression” of the CAR is meant that the expression of the Rab5 or Rab11 protein prevents, opposes, or reduces processes which would normally reduce the presence of CAR complexes at the cell surface. Examples of mechanisms which would prolong the surface expression of CARs include, but are not limited to, enhancing trafficking, reduced endocytosis, or increased recycling of endocytosed receptors back to the cell surface.

It is contemplated that any CAR construct which is expressed in T cells and is used to target other cells could benefit from the methods of the invention. In certain embodiments, the CAR construct is a single fusion polypeptide wherein the Rab5 or Rab11 peptide domain is linked to the CAR construct by a self-cleavable linker sequence such that the CAR/Rab fusion polypeptide is expressed as a single polypeptide and then cleaved into independent proteins.

### Rab Family Proteins and Receptor Expression

Rab family proteins, including Rab5 and Rab11, play a crucial role in endocytosis and membrane transport. Both Rab5 and Rab11 are small guanosine triphosphatase (GTPase) that belongs to the Ras-like GTPase superfamily. Rab5 and Rab11 proteins alternate between an activated (GTP-bound) state and an inactivated (GDP-bound) state. In the activated-state, Rab5 recruits its effectors and regulates the internalization and trafficking of membrane receptors by regulating vesicle fusion and receptor sorting in the early endosomes. Rab5 localizes to early endosomes where it is involved in the recruitment of RAB7A and the maturation of these compartments to late endosomes. Rab5 drives the maturation of endosomes by transporting vacuolar (H+)-ATPases (V-ATPases) from the trans-Golgi network to endocytic vesicles. Rab5 also plays a role in several processes, including amyloid-beta clearance by transcytosis; early endosome to late endosome transport; and regulation of exocytosis. The Rab5 protein is expressed in several cellular components, including the cytoplasmic side of the early endosome membrane. As such, Rab5 is a key regulator in the fusion of endosomes and phagosomes in the early process of phagocytosis and endocytosis and mediates traffic from the plasma membrane to the early endosomes.

While Rab5 appear crucial for early endosome formation, Rab11 has been implicated in the protein recycling pathways. Rab11 exists as a subfamily of two proteins, Rablla and Rab11b, that are closely related. Rab11 proteins have been shown to play a role in protein trafficking from endosomes to the plasma membrane and trans-Golgi network, as well as for polarized transport in epithelial cells, insulin dependent GLUT4 transport and phagocytosis. Both members of Rab11 family proteins are ubiquitously expressed in every cell type and interact with effector proteins that include FP2, FIP3, FIP4, Rab coupling protein (RCP) and RIP11. Some viruses, which assemble in the cytoplasm before being transported to the cell surface, such as the Andes virus (ANDV), use Rab11 mediated transport for delivery to the cell surface and uses both trans-Golgi and Rab11 positive vesicular compartments. Rab11 is also important for transport components of HIV-1, respiratory syncytial virus (RSV) and Mason-Pfizer monkey virus. Recent studies shown a critical role of Rab5 and Rab11 in HSV-1 infection. Combined, siRNA-mediated depletion of Rab5 and Rab11 decreases virus production by 99% in Hela cells. Ultrastructural analysis of HSV-1 infected cells revealed that Rab GTPases 5 and 11 regulated the endocytic tubules used for capsids secondary envelopment.

In some embodiments, the co-expression of Rab5 or Rab11 or Rab5 and Rab11 with the CAR constructs provided herein helps maintain CAR expression and persistence at the surface of the T cell, thereby maintaining or improving CAR T cell function, and by extension, treatment efficacy. In certain embodiments, increased levels of Rab5 expression provided by the vectors or fusion polypeptides of the invention enhance the recycling of endocytosed CAR complexes back to the membrane of the CAR T cell, thereby improving CAR membrane expression persistence and CAR T cell cytotoxicity. In certain embodiments, the expression of Rab5 also opposes or reduces or ameliorates the phenomenon by which CARs are “ripped off”′ the T cells and endocytosed by tumor cells following antigen recognition and binding.

### Fusion Polypeptides and CARs

Also provided herein are fusion polypeptides comprising Rab5 protein domains separated from a CAR domain by a cleavable linker domain. In certain embodiments, the cleavable linker domain comprises a T2A or 2A self-cleaving peptide domain. In certain embodiments, the T2A domain comprises the amino acid sequence set forth in SEQ ID NO: 10.

In certain embodiments, the CAR domain and Rab5 protein domain are expressed separately from each other, for example by being operably linked to different promoters. In certain embodiments, the CAR domain and Rab5 protein domains are comprised within the same nucleic acid molecule but separated by a nucleic acid sequence encoding an internal ribosome entry site or IRES, which permits the expression of the CAR and Rab5 protein domains as distinct, individual polypeptides. It is also contemplated that the CAR construct and the Rab5 protein can be provided by separate expression plasmids. By way of a non-limiting example, the two plasmids may be expressed within the same immune cell such that both the CAR construct and Rab5 protein are expressed with in the same immune cell.

In certain embodiments, the CAR construct comprises antigen recognition domains that specifically bind to tumor-associated antigens. In certain embodiments, the tumor-associated antigen is human CD19. In certain embodiments, the tumor-associated antigen is mesothelin.

Also provided is a chimeric antigen receptor (CAR) construct comprising an antigen binding domain, a transmembrane domain, and an intracellular signaling domain. In certain embodiments, the antigen-binding domain comprises a single chain variable fragment (scFv) comprising an amino acid sequence set forth in SEQ ID NOs: 5-6.

TABLE 1
Example sequences of the invention

| SEQ | | | |
|---|---|---|---|
| ID | | | |
| NO: | Name | Type | Sequence |
| 1. | CD19 | AA | MALPVTALLLPLALLLHAARPEIVMTQSPATLSLSPGERATLSCR |
| | CART | | ASQDISKYLNWYQQKPGQAPRLLIYHTSRLHSGIPARFSGSGSGT |
| | Rab5 | | DYTLTISSLQPEDFAVYFCQQGNTLPYTFGQGTKLEIKGGGGSGG |
| | | | GGSGGGGSQVQLQESGPGLVKPSETLSLTCTVSGVSLPDYGVSWI |
| | | | RQPPGKGLEWIGVIWGSETTYYQSSLKSRVTISKDNSKNQVSLKL |
| | | | SSVTAADTAVYYCAKHYYYGGSYAMDYWGQGTLVTVSSTTTP |
| | | | APRPPTPAPTIASQPLSLRPEACRPAAGGAVHTRGLDFACDIYIWA |
| | | | PLAGTCGVLLLSLVITLYCKRGRKKLLYIFKQPFMRPVQTTQEED |
| | | | GCSCRFPEEEEGGCELRVKFSRSADAPAYKQGQNQLYNELNLGR |
| | | | REEYDVLDKRRGRDPEMGGKPRRKNPQEGLYNELQKDKMAEA |
| | | | YSEIGMKGERRRGKGHDGLYQGLSTATKDTYDALHMQALPPRS |
| | | | GGSGEGRGSLLTCGDVEENPGPRMAGRGGARRPNGPAAGNKIC |
| | | | QFKLVLLGESAVGKSSLVLRFVKGQFHEYQESTIGAAFLTQTVCL |
| | | | DDTTVKFEIWDTAGQERYHSLAPMYYRGAQAAIVVYDITNTDTF |
| | | | ARAKNWVKELQRQASPNIVIALAGNKADLASKRAVEFQEAQAY |
| | | | ADDNSLLFMETSAKTAMNVNEIFMAIAKKLPKNEPQNATGAPGR |
| | | | NRGVDLQENNPASRSQCCSN |
| 2. | M5 | AA | MALPVTALLLPLALLLHAARPEQVQLVQSGAEVEKPGASVKVSC |
| | CART | | KASGYTFTDYYMHWVRQAPGQGLEWMGWINPNSGGTNYAQKF |
| | Rab5 | | QGRVTMTRDTSISTAYMELSRLRSDDTAVYYCASGWDFDYWGQ |
| | | | GTLVTVSSGGGGSGGGGSGGGGSGGGGSDIVMTQSPSSLSASVG |
| | | | DRVTITCRASQSIRYYLSWYQQKPGKAPKLLIYTASILQNGVPSRF |
| | | | SGSGSGTDFTLTISSLQPEDFATYYCLQTYTTPDFGPGTKVEIKTT |
| | | | TPAPRPPTPAPTIASQPLSLRPEACRPAAGGAVHTRGLDFACDIYI |
| | | | WAPLAGTCGVLLLSLVITLYCKRGRKKLLYIFKQPFMRPVQTTQE |
| | | | EDGCSCRFPEEEEGGCELRVKFSRSADAPAYKQGQNQLYNELNL |
| | | | GRREEYDVLDKRRGRDPEMGGKPRRKNPQEGLYNELQKDKMA |
| | | | EAYSEIGMKGERRRGKGHDGLYQGLSTATKDTYDALHMQALPP |
| | | | RSGGSGEGRGSLLTCGDVEENPGPRMAGRGGARRPNGPAAGNKI |
| | | | CQFKLVLLGESAVGKSSLVLRFVKGQFHEYQESTIGAAFLTQTVC |
| | | | LDDTTVKFEIWDTAGQERYHSLAPMYYRGAQAAIVVYDITNTDT |
| | | | FARAKNWVKELQRQASPNIVIALAGNKADLASKRAVEFQEAQA |
| | | | YADDNSLLFMETSAKTAMNVNEIFMAIAKKLPKNEPQNATGAPG |
| | | | RNRGVDLQENNPASRSQCCSN |
| 3. | Rab5 | AA | MAGRGGARRPNGPAAGNKICQFKLVLLGESAVGKSSLVLRFVKG |
| | | | QFHEYQESTIGAAFLTQTVCLDDTTVKFEIWDTAGQERYHSLAP |
| | | | MYYRGAQAAIVVYDITNTDTFARAKNWVKELQRQASPNIVIALA |
| | | | GNKADLASKRAVEFQEAQAYADDNSLLFMETSAKTAMNVNEIF |
| | | | MAIAKKLPKNEPQNATGAPGRNRGVDLQENNPASRSQCCSN |
| 4. | CD8 | A.A | MALPVTALLLPLALLLHAARPE |
| | Leader | | |
| 5. | CD8 | AA | TTTPAPRPPTPAPTIASQPLSLRPEACRPAAGGAVHTRGLDFACDI |
| | Hinge/ | | YIWAPLAGTCGVLLLSLVITLYC |
| | TM | | |
| 6. | CTL119 | AA | IVMTQSPATLSLSPGERATLSCRASQDISKYLNWYQQKPGQAPRL |
| | scFv | | LIYHTSRLHSGIPARFSGSGSGTDYTLTISSLQPEDFAVYFCQQGN |
| | | | TLPYTFGQGTKLEIKGGGGSGGGGSGGGGSQVQLQESGPGLVKP |
| | | | SETLSLTCTVSGVSLPDYGVSWIRQPPGKGLEWIGVIWGSETTYY |
| | | | QSSLKSRVTISKDNSKNQVSLKLSSVTAADTAVYYCAKHYYYGG |
| | | | SYAMDYWGQGTLVTVSS |
| 7. | M5 scFv | AA | QVQLVQSGAEVEKPGASVKVSCKASGYTFTDYYMHWVRQAPG |
| | | | QGLEWMGWINPNSGGTNYAQKFQGRVTMTRDTSISTAYMELSR |
| | | | LRSDDTAVYYCASGWDFDYWGQGTLVTVSSGGGGSGGGGSGG |
| | | | GGSGGGGSDIVMTQSPSSLSASVGDRVTITCRASQSIRYYLSWYQ |
| | | | QKPGKAPKLLIYTASILQNGVPSRFSGSGSGTDFTLTISSLQPEDFA |
| | | | TYYCLQTYTTPDFGPGTKVEIK |
| 8. | 41BB | AA | KRGRKKLLYIFKQPFMRPVQTTQEEDGCSCRFPEEEEGGCE |
| | ICD | | |
| 9. | CD3zeta | AA | LRVKFSRSADAPAYKQGQNQLYNELNLGRREEYDVLDKRRGRD |
| | | | PEMGGKPRRKNPQEGLYNELQKDKMAEAYSEIGMKGERRRGKG |
| | | | HDGLYQGLSTATKDTYDALHMQALPPR |
| 10. | T2A | AA | SGGSGEGRGSLLTCGDVEENPGPR |
| | Domain | | |
| 35. | Rab 11 | | MGTRDDEYDYLFKVVLIGDSGVGKSNLLSRFTRNEFNLESKSTIG |
| | | | VEFATRSIQVDGKTIKAQIWDTAGQERYRAITSAYYRGAVGALL |
| | | | VYDIAKHLTYENVERWLKELRDHADSNIVIMLVGNKSDLRHLRA |
| | | | VPTDEARAFAEKNGLSFIETSALDSTNVEAAFQTILTEIYRIVSQK |
| | | | QMSDRRENDMSPSNNVVPIHVPPTTENKPKVQCCQNI |

### Chimeric Antigen Receptors

The present invention provides compositions and methods for modified immune cells or precursor cells thereof, e.g., modified T cells, comprising a fusion polypeptide comprising chimeric antigen receptors (CARs) and Rab5 or Rab11 proteins separated by a cleavable linker.

Generally speaking, CAR constructs comprise extracellular antigen recognition or binding domains functionally associated with one or more intracellular signaling domains. Therapies comprising CAR-expressing modified T cells are useful to affect the killing of cancer cells whether by direct cytotoxicity or by initiating or amplifying endogenous immune response mechanisms.

In certain embodiments, the invention provides T-cells engineered to express one or more chimeric antigen receptors (CARs) that specifically bind to a tumor antigen for use in cancer immunotherapy. In certain embodiments, the tumor antigen is selected from a group consisting of: TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. A subject CAR of the invention comprises an antigen binding domain (e.g., CD19 binding domain), a transmembrane domain, a costimulatory signaling domain, and an intracellular signaling domain. A subject CAR of the invention may optionally comprise a hinge domain. Accordingly, a subject CAR of the invention comprises an antigen binding domain (e.g., CD19 binding domain), a hinge domain, a transmembrane domain, a costimulatory signaling domain, and an intracellular signaling domain. In some embodiments, each of the domains of a subject CAR is separated by a linker.

The antigen binding domain may be operably linked to another domain of the CAR, such as the transmembrane domain, the costimulatory signaling domain or the intracellular signaling domain, each described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding a costimulatory signaling domain.

The antigen binding domains described herein, including antibodies or antigen-binding fragments thereof, scFvs, and the like, can be combined with any of the transmembrane domains, any of the costimulatory signaling domains, any of the intracellular signaling domains, or any of the other domains described herein that may be included in a CAR of the present invention.

In certain embodiments, the genetically modified immune cell is a T cell.

Accordingly, in one exemplary embodiment, provided herein is a genetically modified T cell comprising a chimeric antigen receptor (CAR) that specifically binds human CD19, comprising: a human CD19 specific antigen binding domain, an optional hinge domain, a transmembrane domain, a costimulatory signaling domain, and an intracellular signaling domain.

In another exemplary embodiment, provided herein is a genetically modified T cell comprising a chimeric antigen receptor (CAR) that specifically binds mesothelin, comprising: a mesothelin specific antigen binding domain, an optional hinge domain, a transmembrane domain, a costimulatory signaling domain, and an intracellular signaling domain.

Sequences of individual domains of the CARs of the current invention are found in Tables 1 and 2.

Nonlimiting examples of CARs of the current invention comprise amino acid sequences set forth in SEQ ID NOs 1-2 and 43-85.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g., a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

In certain embodiments, the CAR of the invention comprises an antigen binding domain that binds to a tumor associated antigen. In certain exemplary embodiments, the antigen binding domain is an scFv antibody that binds to. The choice of antigen binding domain depends upon the type and number of antigens that are present on the surface of a target cell. For example, the antigen binding domain may be chosen to recognize an antigen that acts as a cell surface marker on a target cell associated with a particular status of the target cell.

As described herein, a CAR of the present invention having affinity for a specific target antigen on a target cell may comprise a target-specific binding domain. In some embodiments, the target-specific binding domain is a human target-specific binding domain, e.g., the target-specific binding domain is of human origin. In an exemplary embodiment, a CAR of the present invention having affinity for hCD19 on a target cell may comprise a hCD19 binding domain. In another exemplary embodiment, a CAR of the present invention having affinity for mesothelin on a target cell may comprise a mesothelin binding domain.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In some embodiments, the antigen binding domain is selected from the group consisting of an antibody, an antigen binding fragment (Fab), and a single-chain variable fragment (scFv).

As used herein, the term “single-chain variable fragment” or “scFv” is a fusion protein of the variable regions of the heavy (VH) and light chains (VL) of an immunoglobulin (e.g., mouse or human) covalently linked to form a VH:VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., hCD19 or mesothelin binding domain) comprises an scFv having the configuration from N-terminus to C-terminus, VH—linker—VL. In some embodiments, the antigen binding domain (e.g., hCD19 or mesothelin binding domain) comprises an scFv having the configuration from N-terminus to C-terminus, VL—linker—VH. Those of skill in the art would be able to select the appropriate configuration for use in the present invention.

In some embodiments, the hCD19 binding domain is derived from an scFv specific for human CD19 as disclosed elsewhere herein. Accordingly, a CAR of the present invention comprises a hCD19 binding domain derived from an scFv disclosed elsewhere herein.

In some embodiments, the mesothelin binding domain is derived from an scFv specific for human mesothelin as disclosed elsewhere herein. Accordingly, a CAR of the present invention comprises a mesothelin binding domain derived from an scFv disclosed elsewhere herein.

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

The antigen binding domain may be operably linked to another domain of the CAR, such as the transmembrane domain or the costimulatory signaling domain, both described elsewhere herein. In one embodiment, a nucleic acid encoding the antigen binding domain is operably linked to a nucleic acid encoding a transmembrane domain and a nucleic acid encoding a costimulatory signaling domain.

The antigen binding domains described herein, can be combined with any of the transmembrane domains described herein, any of the intracellular domains or cytoplasmic domains described herein, or any of the other domains described herein that may be included in the CAR.

TABLE 2
CAR Construct Features and Exemplary Anti-CD19 CARs

| SEQ | | |
|---|---|---|
| ID | | |
| NO: | Name: | Sequence: |
| 36 | scFv KIR | ATGGCCTTACCAGTGACCGCCTTGCTCCTGCCGCTGGCCTTGCTGCTC |
| | construct | CACGCCGCCAGGCCGGGATCCXGCTAGCGAGCAGAAATTAATCTCA |
| | DNA | GAGGAGGACCTGCCTAGCGGTGGCGGAGGTTCTGGAGGTGGGGGTT |
| | | CCTCACCCACTGAACCAAGCTCCAAAACCGGTAACCCCAGACACCTG |
| | | CATGTTCTGATTGGGACCTCAGTGGTCAAAATCCCTTTCACCATCCTC |
| | | CTCTTCTTTCTCCTTCATCGCTGGTGCTCCAACAAAAAAAATGCTGCT |
| | | GTAATGGACCAAGAGCCTGCAGGGAACAGAACAGTGAACAGCGAGG |
| | | ATTCTGATGAACAAGACCATCAGGAGGTGTCATACGCATAA |
| | | (X indicates scFv insertion site) |
| 37 | scFv KIR | MALPVTALLLPLALLLHAARPGSXASEQKLISEEDLPSGGGGSGGGGSSP |
| | construct | TEPSSKTGNPRHLHVLIGTSVVKIPFTILLFFLLHRWCSNKKNAAVMDQE |
| | AA | PAGNRTVNSEDSDEQDHQEVSYA |
| | | (X indicates scFv insertion site) |
| 38 | Myc tag | GAGCAGAAATTAATCTCAGAGGAGGACCTG |
| 39 | Linker | SGGGGSGGGGS |
| 40 | TM/KIR2 | CCCACTGAACCAAGCTCCAAAACCGGTAACCCCAGACACCTGCATGT |
| | DS2 | TCTGATTGGGACCTCAGTGGTCAAAATCCCTTTCACCATCCTCCTCTT |
| | domains | CTTTCTCCTTCATCGCTGGTGCTCCAACAAAAAAAATGCTGCTGTAAT |
| | | GGACCAAGAGCCTGCAGGGAACAGAACAGTGAACAGCGAGGATTCT |
| | | GATGAACAAGACCATCAGGAGGTGTCATACGCA |
| 41 | TM/KIR2 | PTEPSSKTGNPRHLHVLIGTSVVKIPFTILLFFLLHRWCSNKKNAAVMDQ |
| | DS2 | EPAGNRTVNSEDSDEQDHQEVSYA |
| | domains | |
| 42 | CD8 | MALPVTALLLPLALLLHAARP |
| | Leader | |
| | Sequence | |
| 43 | scFv_01 | MALPVTALLLPLALLLHAARPGSQAVLNQPASVSAALGQRVTISC |
| | CAR | NTNIGSPYDVQWYQQLPGKSPKTIIYGNSNRPSGVPVRESGSKSGS |
| | | TATLTIAGIQAEDEADYYCQSYDDSLDGRVVFGGGTQLTVLGGG |
| | | SSRSSSSGGGGSGGGGEVQLVESGGDLVKPGGSLRLSCVASGFTF |
| | | SNHEMYWVRQAPGKGLEWVARIYDSGSRTRYADAVKGRFTISR |
| | | DNAKNTLYLQMNSLTAEDTAVYFCAGGNSRGWTEFGMDYWGP |
| | | GTLVTVSSASEQKLISEEDLPSGGGGSGGGGSSPTEPSSKTGNPRH |
| | | LHVLIGTSVVKIPFTILLFFLLHRWCSNKKNAAVMDQEPAGNRTV |
| | | NSEDSDEQDHQEVSYA |
| 44 | scFv_02 | MALPVTALLLPLALLLHAARPGSSYVLTQSPSLSGSLGQKVTISCT |
| | CAR | GSRSNLGGNYVGWYQQVPGMAPRNVIYANDFRPSRIPARFSASK |
| | | SGTSATLTISGLQAEDEADYFCSSWDDDLGSHVFGSGTQLTILGG |
| | | GSSRSSSSGGGGSGGGGEEQLVEFGGDLVKPGGSLRLSCVASGFS |
| | | FSSYDMSWVRQAPGKGLQWVASTNFDGSRTYYTDAVEGRFTISR |
| | | DNARNTVYLQMNSLRAEDTAVYYCARGMGSWGASTFGYWGQG |
| | | TLVTVSSASEQKLISEEDLPSGGGGSGGGGSSPTEPSSKTGNPRHL |
| | | HVLIGTSVVKIPFTILLFFLLHRWCSNKKNAAVMDQEPAGNRTVN |
| | | SEDSDEQDHQEVSYA |
| 45 | scFv_03 | MALPVTALLLPLALLLHAARPGSQSVLNQPPSVSGALGQTVTISC |
| | CAR | SGSHTNIGSGFDVQWYQQLPGKSPQTLVYANINRPSGVPARFSGS |
| | | KSGSTATLTITGVQAEDEADYYCQSYDDNFDGHVFGSGTQLTVL |
| | | GGGSSRSSSSGGGGSGGGGEVQLVQSGGDLVKPGGSLRLSCVAS |
| | | GFTFSNYTMSWVRQAPGQGLQWVAYINYDGSITYYAEAVKGRF |
| | | TVSRDNAKNTLYLQMNSLRAEDTAVYFCVKETFMIPTEWGQGTL |
| | | ITVSSASEQKLISEEDLPSGGGGSGGGGSSPTEPSSKTGNPRHLHVL |
| | | IGTSVVKIPFTILLFELLHRWCSNKKNAAVMDQEPAGNRTVNSED |
| | | SDEQDHQEVSYA |
| 46 | scFv_04 | MALPVTALLLPLALLLHAARPGSQSVLNQPPSVSGALGQTVTISC |
| | CAR | SGSHTNIGSGFDVQWYQQLPGKSPQTIIYANINRPSGVPARFSGSK |
| | | SGSTATLTITGVQAEDEADYYCQSYDDNFDYVFGSGTQLTVLGG |
| | | GSSRSSSSGGGGSGGGGEEQLVEFGGDLVKPGGSLRLSCVVSGFD |
| | | FSRYGMSWVRQSPGKGLQWVADISYRGTTFYADDVKGRFTVSR |
| | | DSAKNTLYLEMTSLTVEDTAVYYCANSANWYVSPFDHWGQGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 47 | scFv_05 | MALPVTALLLPLALLLHAARPGSQPVLTQPPSVSGSLGQRVTISCS |
| | CAR | GSTNTIGVVGANWYQQLPGKSPKLLVYSDGVRPSGVPDRFSGSR |
| | | SANSDTLTITGLQAEDEADYYCQSFDSTHNAIVFGGGTHLTVLGG |
| | | GSSRSSSSGGGGSGGGGEVQLVETGGDLVKPAGSLRLSCAASGFP |
| | | FSGYSMTWVRQAPEKGLQLVAGINSDGSHTYYTDDVKGRFTISR |
| | | DNTKYILYLQMNSLRAEDTAMYYCGSHSWGNFHYWGQGTLVT |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 48 | scFv_06 | MALPVTALLLPLALLLHAARPGSQPVLTQPPSVSGSLGQRVTISCT |
| | CAR | GTNTNIGSGYSVQWYQQLPGESPKPIIYGSSNRPSGVPARFSGSKS |
| | | GSTGTLTITGIQAEDEADYHCQSYDDSLDGHAVFGGGTQLTILGG |
| | | GSSRSSSSGGGGSGGGGEVQLVQSGGDLVKPGGSLRLSCVASGFT |
| | | FSDNYMSWIRQVPGKGLQWVSRISTDGRGTSYADAVKGRFTISR |
| | | DNAKNTLYLQMNSLRDEDTAVYYCARRSGIWGFDYWGQGALV |
| | | TVSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 49 | scFv_07 | MALPVTALLLPLALLLHAARPGSSYELTQPPSMSAALGQRVTISC |
| | CAR | TGSNTNIGSGYDVQWYQQVPGKSPTPLIYGNTNRPSGVPVRFSGS |
| | | KSGNTATLTITGIQAEDEADYYCQSYDDNFDGHHVFGSGTQLTV |
| | | LGGGSSRSSSSGGGGSGGGGEVQLVESGGDLVKPAGSLRLSCVAS |
| | | GFTFSNYGMNWVRQAPGKGLQWVAGINSGGSTTTYADVVKGRF |
| | | TISRDNAKNTLYLQMNSLGAEDTAVYYCARVDYSSSWLVDYWG |
| | | QGTLVTVSSASEQKLISEEDLPSGGGGSGGGGSSPTEPSSKTGNPR |
| | | HLHVLIGTSVVKIPFTILLFFLLHRWCSNKKNAAVMDQEPAGNRT |
| | | VNSEDSDEQDHQEVSYA |
| 50 | scFv_08 | MALPVTALLLPLALLLHAARPGSQSVLTQPASVSGSLGQKVTISC |
| | CAR | TGSRSNIGANYVGWYQQVPGIGPRTVIFGYNYRPSGVPDRESSSK |
| | | SGNSATLTISGLQAEDEAEYYCSSWDDSLRGTVFGSGTQLTVLGG |
| | | GSSRSSSSGDGGSGGGGEVQLVESGGDLVKPGESLRLSCVASGFT |
| | | FSSYGMSWVRQSPGKGLQWVADISYGGGTYYADAVKGRFTISR |
| | | DNARNTVYLQMNSLRAEDTAVYYCASQWGDWGHFEYWGQGT |
| | | LVTVSSASEQKLISEEDLPSGGGGSGGGGSSPTEPSSKTGNPRHLH |
| | | VLIGTSVVKIPFTILLFELLHRWCSNKKNAAVMDQEPAGNRTVNS |
| | | EDSDEQDHQEVSYA |
| 51 | scFv_09 | MALPVTALLLPLALLLHAARPGSQPVLTQPPSVSAALGQRVTLSC |
| | CAR | TGSKTNIGSGYDVQWYQQFPGKSPKTIIYGNSNRFSGVPARFSGS |
| | | KSGSIATLTITGIQAEDEADYYCQSFDDNVDGYVFGSGTQLTILGG |
| | | GSSRSSSSGGGGSGGGGEVQLVESGGDLVKPGGSLRLSCVASGFT |
| | | FSSDSIHWVRQAPGKGLQWVAYINSGGSTTYYADAVKGRFTISR |
| | | DNARNTVYLKMNSLRAEDTAVYYCAKDSGFGSWYLESWGQGT |
| | | LVTVSPASEQKLISEEDLPSGGGGSGGGGSSPTEPSSKTGNPRHLH |
| | | VLIGTSVVKIPFTILLFFLLHRWCSNKKNAAVMDQEPAGNRTVNS |
| | | EDSDEQDHQEVSYA |
| 52 | scFv_10 | MALPVTALLLPLALLLHAARPGSQAVLNQPASVSGALGQTVTISC |
| | CAR | SGSHTNIGSGFDVQWYQQLPGKSPQTIIYANINRPSGVPARFSGSK |
| | | SGSTATLTITGVQAEDEADYHCQSFDDDLDGPVFGGGTHLTVLG |
| | | GGSSRSSSSGGGGSGGGGEVQLVQSGGDLVKPGGSLKISCVASGL |
| | | TFNRNYMTWIRQAPGKGLQWVSEINPDGSTTSYTDAVRGRFTISR |
| | | DNAKNTLYLQMNSMRDEDTAVYYCARSSWGSFDYWGQGTLVT |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 53 | scFv_11 | MALPVTALLLPLALLLHAARPGSQPVLTQPPSVSGALGQTVTISCS |
| | CAR | GSHTNIGSGFDVQWYQQLPGKSPQTLVYANINRPSGVPARFSGSK |
| | | SGSTATLTITGVQAEDEADYYCQSYDDNFDYVFGSGTQLTVLGG |
| | | GSSRSSSSGGGGSGGGGEVQLVESGGDLVKPGGSLRLSCVASGFT |
| | | FSDYSMSWVRQSPGKGLQWVAAIMTSGSTYYADAVKGRFTISRD |
| | | NAKNTLYLQMDSLRAEDTAVYYCAHDYWFDFWGQGTLVTVSS |
| | | ASEQKLISEEDLPSGGGGSGGGGSSPTEPSSKTGNPRHLHVLIGTS |
| | | VVKIPFTILLFELLHRWCSNKKNAAVMDQEPAGNRTVNSEDSDE |
| | | QDHQEVSYA |
| 54 | scFv_12 | MALPVTALLLPLALLLHAARPGSSYELTQPPSATVTLRQTARLTC |
| | CAR | GGENIGRKSVQWYQQKPGQSPLLIIYADSSRPSGIPERFSGANSGN |
| | | TASLTISGALAEDEADYYCQIWDRNINVFGSGTQLTVLGGGSSRS |
| | | SSSGGGGSGGGGEEQLVEFGGDLVKPAGSLRLSCVASGFTFSSYD |
| | | MSWVRQAPGKGLQWVATISYDGSSTYYTDAVKGRFTVSRDNAK |
| | | NTLYLQMNSLRAEDTALYYCAKDSGSWSHFYGLDTWGHGTSVF |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 55 | scFv_13 | MALPVTALLLPLALLLHAARPGSQPVLTQPPSVSGALGQTVTISCS |
| | CAR | GSHTNIGSGFDVQWYQQLPGKSPQTLVYANINRPSGVPARFSGSK |
| | | SGSTATLTITGVQAEDEADYYCQSYDDNFDYVFGSGTQLTVLGG |
| | | GSSRSSSSGDGGSGGGGEVQLVESGGGLMKPGGSLKLSCLASGFT |
| | | FSRYYMYWVRQAPGKGLQWVAQISSDGRRTYYADAVQGRFTIS |
| | | RDNVKNTVYLQMNSLSDEDTAVYYCVREDYFDFDYWGQGTLV |
| | | TVSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 56 | scFv_14 | MALPVTALLLPLALLLHAARPGSQSVLNQPPSVSAALGQRITISCT |
| | CAR | GSHTNIGSGYDVQWYQQLPGKSPRTIIYGNSDRPSGVPVRFSGSK |
| | | SGNTATLTITGIQAEDEADYYCQSYDDNLNDPYVFGSGTQLTVLG |
| | | GGSSRSSSSGGGGSGGGGEVQLVQSGGDLVKPGGSLRLSCVASG |
| | | FAFSGKTMQWVRQAPGKGLQWVAYIQHDGSRTSYADAVKGRFT |
| | | ISRDNAKNTLYLQMDSLRAEDTAVYYCADHPWYGPEYWGQGTP |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 57 | scFv_15 | MALPVTALLLPLALLLHAARPGSSYELTQPPSVSAALGQGVTISCI |
| | CAR | GSDTNIGNHYDVQWYQQLPGKSPRTIIYGNSDRPSGVPGRFSGSK |
| | | SGNTATLTITGIQAEDEADYHCQSYDDDLDGHIVFGGGTHLTVLG |
| | | GGSSRSSSSGGGGSGGGGEVQLVQSGGDLVKPGGSLRLSCVASG |
| | | FTFNKYHMSWVRQAPGKGLQWVGKITSDGSATGYADAVKGRFT |
| | | ISRDNAKNTFYLQMDSLRAEDTAMYYCARDQSSWGTGNFDYWG |
| | | QGTLVTVSSASEQKLISEEDLPSGGGGSGGGGSSPTEPSSKTGNPR |
| | | HLHVLIGTSVVKIPFTILLFFLLHRWCSNKKNAAVMDQEPAGNRT |
| | | VNSEDSDEQDHQEVSYA |
| 58 | scFv_16 | MALPVTALLLPLALLLHAARPGSQSVLTQPTSVSGSLGQRVTISCT |
| | CAR | GSSSNVGYGNYVGWYQQLPGTGPRTLIYGSHYRPSGVPDRFSGSS |
| | | SGSSATLTISGLQAEDEADYYCSSYDSTLSGYVFGSGTQLTILGGG |
| | | SSRSSSSGGGGSGGGGEVQLVESGGDLVKPGGSLRLSCVGSGFTL |
| | | SSYWMSWVRQAPGKGLQWIAEISGSGNTIYYGDAVKGRFTISRD |
| | | NAKNTLYLQMNSLRDEDTAVYYCATEYYWNIDNWGQGTLVTV |
| | | SSASEQKLISEEDLPSGGGGSGGGGSSPTEPSSKTGNPRHLHVLIGT |
| | | SVVKIPFTILLFELLHRWCSNKKNAAVMDQEPAGNRTVNSEDSDE |
| | | QDHQEVSYA |
| 59 | scFv_17 | MALPVTALLLPLALLLHAARPGSQPVLTQPPSVSAALGQRVTISC |
| | CAR | TGSKTNIGSGYDVQWYQQLPGKSPKTIIYGNSNRPSGVPARFSGS |
| | | KSGSIATLTITGIQAEDEADYYCQSFDDNVDGYVFGSGTQLTILGG |
| | | GSSRSSSSGGGGSGGGGEVPLVESGGDLVKPGGSLRLSCVASGFT |
| | | FSSYGMSWVRQAPGKGLQWVAYINGGGSSTSYADAVRGRFTISR |
| | | DNAENTLYLQMNSLRADDTAVYYCSNYYGIYWDSDYWGQGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 60 | scFv_18 | MALPVTALLLPLALLLHAARPGSQSALTQTASVSGSLGQKVTISC |
| | CAR | TGSRSNIGANYVGWYQQVPGIGPRTVIFGYNYRPSGVPDRESSSK |
| | | SGNSATLTISGLQAEDEAEYYCSTWDSSLNAYVFGSGTQLTVLGG |
| | | GSSRSSSSGGGGSGGGGEVPLVESGGDLVKPGGSLRLSCVASGFT |
| | | LSNYGMSWVRQSPGKGLQWVAAIRSGGNTYYADAVKGRFTISR |
| | | DNAKNTLYLQMNSLRAEDTAVYYCASQWGDWGHFEYWGQGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 61 | scFv_19 | MALPVTALLLPLALLLHAARPGSQAVLTQPASVSGPLGQRVTISC |
| | CAR | SGSTNNIGIVGAAWYQQFPGKAPKLLVYSDGSRPSGVPDRFSGSN |
| | | SGNSATLTISGLQAEDEADYYCQSVDATHGTILFGGGTQLTVLGG |
| | | GSSRSSSSGGGGSGGGGEVQLVETGGDLVKPAGSLRLSCAASGFP |
| | | FSGYSMTWVRQAPEKGLQLVAGINSDGSHTYYTDDVKGRFTVSR |
| | | DNTKYILYLQMNSLRVEDTAIYYCGSHSWGNFHYWGQGTLVTV |
| | | SSASEQKLISEEDLPSGGGGSGGGGSSPTEPSSKTGNPRHLHVLIGT |
| | | SVVKIPFTILLFFLLHRWCSNKKNAAVMDQEPAGNRTVNSEDSDE |
| | | QDHQEVSYA |
| 62 | scFv_20 | MALPVTALLLPLALLLHAARPGSQSVLNQPPSVSVSLGQTATISCS |
| | CAR | GESLSKYYAQWFQQKAGQVPVLVIYKDTERPSGIPDRESGSSSGN |
| | | THTLTISGARAEDEADYYCESEVSPDTIVFGGGTHLTVLGGGSSRS |
| | | SSSGGGGSGGGGEVQLVESGGDLVKPGGSLRLSCVASGFTFSSYA |
| | | MSWVRQAPGEGLQWVAYINSAGSTTYYADAVKGRFTISRDNAR |
| | | NTVYLQMNSLRAEDTAVYYCGRYYYGLDYWGPGTSVFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 63 | scFv_21 | MALPVTALLLPLALLLHAARPGSQPVLTQPPSVSVSLGQTATISCS |
| | CAR | GESLSKYYAQWFQQKAGQAPVLVIYKDTERPSGIPDRFSGSSSGN |
| | | THTLTISGARAEDEADYYCESAVSSDTAVFGSGTQLTVLGGGSSR |
| | | SSSSGGGGSGGGGEVQLVESGGDMVKPGGSLRLSCVASGFTFSSF |
| | | WMYWVRQAPGKGLQWVGGINSGGSITSYADAVKGRFTISRDNA |
| | | RNTLYLQMDSLRDEDTAVYYCAKPDIYGSFDHWGQGTLVTVSS |
| | | ASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 64 | scFv_22 | MALPVTALLLPLALLLHAARPGSQPVLTQPPSVSGSLGQRVTISCT |
| | CAR | GSSSNVGYGDYVGWYQQLPGTSPRTLIYSSSSRPSGVPDRESGSRS |
| | | GSTATLTISGLQAEDEADYHCSSYDTSLNGFVFGSGTQLTILGGGS |
| | | SRSSSSGGGGSGGGGELTLQESGPGLVKPAQTLSLTCTVSGDSVT |
| | | SSHFWHWIRQRPGRGLEWMGYWSDITIYSPTFQGRISITVDTARN |
| | | QFSLHLNSVTTEDSAVYFCARYNGYVDYLDVWGQGTLITVSSAS |
| | | EQKLISEEDLPSGGGGSGGGGSSPTEPSSKTGNPRHLHVLIGTSVV |
| | | KIPFTILLFFLLHRWCSNKKNAAVMDQEPAGNRTVNSEDSDEQD |
| | | HQEVSYA |
| 65 | scFv_23 | MALPVTALLLPLALLLHAARPGSDIVMTQTPLSLSVSPGEPASISC |
| | CAR | KASQSLLHSNGNTYLYWFRQKPGQSPQRLIYLVSNRDAGVPDRF |
| | | SGSGSGTDFTLRISRVEADDSGIYYCGQGKQDPITFGEGTHLEIKG |
| | | GSSRSSSSGGGGSGGGGELTLQESGPGLVKPSQTLSLTCVVSGGS |
| | | VTTSDYWNWLRQRPGRGLEYMGYWKGRTEYNPAFQGRISITAD |
| | | TAKNQFSLQLNSVTADDTAIYYCAHWGSGNLDYWGPGTLVTVS |
| | | SASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 66 | scFv_24 | MALPVTALLLPLALLLHAARPGSQSVLNQPPSVSGALGQTVTISC |
| | CAR | SGSHTNIGSGFDVQWYQQLPGKSPQTLVYANINRPSGVPVRFSGS |
| | | KSGTTATLTITGIQAEDEADYYCQSYDDNVDGYVFGSGTQLTVL |
| | | GGGSSRSSSSGGGGSGGGGEVQLVQSGGXLVKPGGSLRLSCVAS |
| | | GFTLSSNYMDWVRQAPGKGPQWVAQISTDGSRTYYADAVKGRF |
| | | TISRDTAKNTLYLQMNSLRAEDTAMYYCAKNEYLAPTYWGQGT |
| | | LVTVSSASEQKLISEEDLPSGGGGSGGGGSSPTEPSSKTGNPRHLH |
| | | VLIGTSVVKIPFTILLFFLLHRWCSNKKNAAVMDQEPAGNRTVNS |
| | | EDSDEQDHQEVSYA |
| 67 | scFv_25 | MALPVTALLLPLALLLHAARPGSDIVMTQTPLSLSVSPGETASISC |
| | CAR | KASQSLLHSNGNTYLNWFRQKPGQSPQVLIYQVSNRDTGVPDRF |
| | | SGSGSGTDFTLRISRVEADDAGVYYCGQGIQYPPTFGAGTKVELK |
| | | GGSSRSSSSGGGGSGGGGEEQLVEFGGDLVKPGGSLRLSCVASGF |
| | | TFSSLSMNWIRQAPGKGLQWVSQISPDGTITGYTDAVKGRFTISR |
| | | DNAKNTLYLQMNSLRDEDTAIYYCAKSRYTDTYPFDYWGPGTL |
| | | VTVSSASEQKLISEEDLPSGGGGSGGGGSSPTEPSSKTGNPRHLHV |
| | | LIGTSVVKIPFTILLFFLLHRWCSNKKNAAVMDQEPAGNRTVNSE |
| | | DSDEQDHQEVSYA |
| 68 | scFv_26 | MALPVTALLLPLALLLHAARPGSSSVLTQPPSVSVSLGQTATISCS |
| | CAR | GESLSKYYTQWFQQKAGQAPILVIYKDTERPSGIPDRFSGSSSGNT |
| | | HTLTISGARAEDEADYYCESLVDSDAYMFGSGTQLTVLGGGSSRS |
| | | SSSGGGGSGGGGEVQLVETGGDLVKPGGSLRLSCVGSGFTFSSY |
| | | WMSWVRQAPGKGLQWIADISDSGSSTDYADAVKGRFTISRDNA |
| | | KNTLYLQMNSLRAEDTAMYYCARGLGMDYWGHGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 69 | scFv_27 | MALPVTALLLPLALLLHAARPGSQPVLTQPPSVSVSLGQTATISCS |
| | CAR | GESLSKYYAQWFQQKAGQAPVLVIYKDTERPSGIPDRFSGSSSGN |
| | | THTLTITGIQAEDEGDYHCHSYDENLDGYVFGSGTQLTVLGGGSS |
| | | RSSSSGGGGSGGGGEVQLVESGGDLVKPGGSLRLSCVGSGFTFSS |
| | | YWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 70 | scFv_28 | MALPVTALLLPLALLLHAARPGSQSALTQPSSVSVSLGQTATISCS |
| | CAR | GESLNKYYVQWFQQKAGQVPILVIYRDTERPSGIPDRFSGSSSGN |
| | | THTLTITGTRAEDEADYYCESLVNDNTYVFGSGTQLTVLGGGSSR |
| | | SSSSGGGGSGGGGEVQLVETGGDLVKPGGSLRLSCVGSGFTFSSY |
| | | WMSWVRQAPGKGLQWIADISDSGSSTDYADAVKGRFTISRDNA |
| | | KNTLYLQMNSLRAEDTAMYYCARGLGMDYWGHGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 71 | scFv_29 | MALPVTALLLPLALLLHAARPGSQSVLNQPPSVSVSLGQTATISCS |
| | CAR | GESLNKYYTQWFQQKAGQAPVLVIYKDTERPSGIPDRFSGSSSGN |
| | | THTLTISGARAEDEADYYCESVDSTDTYVFGSGTQLTILGGGSSRS |
| | | SSSGGGGSGGGGEVHLVESGGDLVKPGGSLRLSCVGSGFTFSSY |
| | | WMSWVRQAPGKGLQWIADISDSGSSTDYADAVKGRFTISRDNA |
| | | KNTLYLQMNSLRAEDTAMYYCARGLGMDYWGHGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 72 | scFv_30 | MALPVTALLLPLALLLHAARPGSQSVLTQPASVSVSLGQTATISCS |
| | CAR | GESLSKYYTQWFQQKAGQAPVLVIYKDTERPSGIPDRFSGSSSGN |
| | | THTLTISGAQAEDEADYYCQSYDDNVDGYVFGSGTQLTVLGGGS |
| | | SRSSSSGGGGSGGGGEVQLVESGGDLVKPGGSLRLSCVGSGFTFS |
| | | SYWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 73 | scFv_31 | MALPVTALLLPLALLLHAARPGSQSALTQPSSVSVSLGQTATISCS |
| | CAR | GESLTKYYVQWFQQKAGQAPVLVIYRDTERPSGIPDRFSGSSSGN |
| | | THTLTISGARAEDEADYYCESMDGREAYVFGSGTQLTVLGGGSS |
| | | RSSSSGGGGSGGGGEVQLVESGGDLVKPGGSLRLSCVASGFTFSS |
| | | YWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 74 | scFv_32 | MALPVTALLLPLALLLHAARPGSQSVLTQPASVSVSLGQTATISCS |
| | CAR | GESLSKYYAQWFQQKAGQAPVLVIYKDTERPSGIPDRFSGSSSGN |
| | | THTLTITGIQAADEADYYCQSYDDNLDGYVFGSGTQLTVLGGGS |
| | | SRSSSSGGGGSGGGGEVQLVESGGDLVKPGGSLRLSCVGSGFTFS |
| | | SYWMSWVRQAPGKGLQWIADISDSGSSTDYADAVKGRFTISRDN |
| | | AKNTLYLQMNSLRAEDTAMYYCARGLGMDYWGHGTSLFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |
| 75 | scFv_33 | MALPVTALLLPLALLLHAARPGSDIVMTQTPLSLSVSPGEPASISC |
| | CAR | KASQSLLHSDGNTYLHWYLQKPGQSPQLLIYSVSNRFTGVSDRFS |
| | | GSGSGTDFTLRISRVEADDTGVYYCGQGTHLPFTFGQGTKLEIKG |
| | | GSSRSSSSGGGGSGGGGEVQLVESGGDLVKPGGSLRLSCAASGFT |
| | | FSHYDMNWVRQAPGKGLQWVASIAYDGSNTYYTDAVKGRFTIS |
| | | RDNAKNILFLLVNSLRAEDTAIYYCARGLVYWGQGTLVTVSSAS |
| | | EQKLISEEDLPSGGGGSGGGGSSPTEPSSKTGNPRHLHVLIGTSVV |
| | | KIPFTILLFFLLHRWCSNKKNAAVMDQEPAGNRTVNSEDSDEQD |
| | | HQEVSYA |
| 76 | scFv_34 | MALPVTALLLPLALLLHAARPGSSYELTQPPSVSAALGQRVTISCT |
| | CAR | GTNTNIGSGYSVQWYQQLPGESPKPIIYGSSNRPSGVPARFSGSKS |
| | | GSTGTLTITGIQAEDEADYHCQSYDDSLDGHAVFGGGTQLTILGG |
| | | GSSRSSSSGGGGSGGGGEVQLVESGGDLVKPGGSLRLSCVASGLT |
| | | FSDNYMSWIRQVPGKGLQWVSRISTDGRGTSYADAVKGRFTISR |
| | | DNAKNTLYLQMNSLRDEDTAVYYCARRSGIWGFDYWGQGTLVT |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 77 | scFv_35 | MALPVTALLLPLALLLHAARPGSSSVLTQPPSVSVSLGQTATISCS |
| | CAR | GESLSEYYAQWFQQKAGQAPVLVIYKDTERASGIPDRESGSSSGN |
| | | THTLTISGARAEDEADYYCESAVNSDGIVFGGGTHLTVLGGGSSR |
| | | SSSSGGGGSGGGGEVQLVESGGDLVKPAGSLRLSCVTSGFTFSSY |
| | | AMSWVRQAPGKGLQWVGSINSGGSTTSYADAVKGRFTISRDNA |
| | | KNTVYLQMNSLRAEDTAVYYCARWWDFEYWGQGTLVTVSSAS |
| | | EQKLISEEDLPSGGGGSGGGGSSPTEPSSKTGNPRHLHVLIGTSVV |
| | | KIPFTILLFFLLHRWCSNKKNAAVMDQEPAGNRTVNSEDSDEQD |
| | | HQEVSYA |
| 78 | scFv_36 | MALPVTALLLPLALLLHAARPGSQSVLTQPTSVSGSLGQRVTISCS |
| | CAR | GSTNNIGIVGASWYQQLPGKAPKLLVYSDGDRPSGVPDRFSGSNS |
| | | GNSDTLTITGLQAEDEADYYCQSFDTTLDAYVFGSGTQLTVLGG |
| | | GSSRSSSSGGGGSGGGGEEQLVEFGGGLVRPGGSLRLSCVASGFT |
| | | FSSYGMTWVRQSPGKGLQWVATISDTGSTYYADTVKGRFSISRD |
| | | NAKNTLYLQMNSLRADDTALYYCTKDLGVWGMDNWGPGTSLF |
| | | VSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 79 | scFv_37 | MALPVTALLLPLALLLHAARPGSSSVLTQPPSVSVSLGQTATISCS |
| | CAR | GESLNDYYAQWFQQKAGQVPVLVIYRDTERPSGIPDRFSGSSSGN |
| | | THTLTISGARAEDEADYYCESEVSAGGAVFGEGTHLTVLGGGSSR |
| | | SSSSGGGGSGGGGEVQLVETGGDLVKPGGSLRLSCVASGFTLRD |
| | | YSMTWVRQTPEKGLQLVAGISTGGSTTYYIDAVKGRFTISRDNAK |
| | | NTVYLQMNSLRAEDTAMYHCTTTALGYWGQGTLVTVSSASEQK |
| | | LISEEDLPSGGGGSGGGGSSPTEPSSKTGNPRHLHVLIGTSVVKIPF |
| | | TILLFELLHRWCSNKKNAAVMDQEPAGNRTVNSEDSDEQDHQEV |
| | | SYA |
| 80 | scFv_38 | MALPVTALLLPLALLLHAARPGSSSVLTQPPSVSVSLGQTATISCS |
| | CAR | GESLSEYYAQWFQQKAGQAPVLVIYKDTERPSGIPDRFSGSSSGN |
| | | THTLTISGARAEDEADYYCASADSSAIPVFGGGTHLTVLGGGSSR |
| | | SSSSGGGGSGGGGEVQLVESGGDLVKPGGSLRLSCVASGFNFGN |
| | | YAVSWVRQAPGKGLQWVAGISNDGTITGYTDVVEGRFIISRDNA |
| | | KNTLYLQMNSLRAEDMAIYYCVAGGFLDYWGQGTLVTVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 81 | scFv_39 | MALPVTALLLPLALLLHAARPGSQSVLTQPPSVSGSPGQRVTISCS |
| | CAR | GTTKNIGIVGASWYQQLPGKAPKLLVHSDGFRPSGVPDRFSASNS |
| | | GDSDILTITGLQAEDEADYYCQSFDTTLDAHVFGSGTQLTVLGGG |
| | | SSRSSSSGGGGSGGGGEVPLVESGGDLVKPGGSLRLSCVASGFTF |
| | | SSYGMSWVRQSPGKGLQWVAGITYDGSNTYYTDTVRGRFTISRD |
| | | NARNTVYLQMNSLRAEDTAVYYCVASPLYGTYGFDYWGHGTSV |
| | | FVSSASEQKLISEEDLPSGGGGSGGGGSSPTEPSSKTGNPRHLHVLI |
| | | GTSVVKIPFTILLFFLLHRWCSNKKNAAVMDQEPAGNRTVNSEDS |
| | | DEQDHQEVSYA |
| 82 | scFv_40 | MALPVTALLLPLALLLHAARPGSQSVLTQPPSVSGSLGQKVTISCT |
| | CAR | GSSSSVGTHYVGWYQIPPGIGPRTVIYNNDYRPSGVPDRFSGSKSG |
| | | NSATLTISGLQAEDEADYYCSSWDDSLGAYVLGSGTQLTILGGGS |
| | | SRSSSSGGGGSGGGGELTLQESGPGLVKPSQTLSLTCAVSGGSVIR |
| | | GYYWTWIRQRPGRGLEWMGYWAGSTDYNPAFQGRIFITAGTAK |
| | | NQFSLEMTSMTTDDTAIYYCVGSLRGGWLFDNWGQGTLVTVSS |
| | | ASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 83 | scFv_41 | MALPVTALLLPLALLLHAARPGSQPVLTQPPSVSVSLGQTATISCS |
| | CAR | GESLSKYYAQWFQQKAGQAPVLVIYKDTERPSGIPDRFSGSSSGN |
| | | THTLTISGARAEDEADYYCESAVSTETVVFGGGTHLTVLGGGSSR |
| | | SSSSGGGGSGGGGEVQLVETGGDLVMPAGSLRLSCVASGFTFSSS |
| | | VMSWVRQAPGKGLEWVAGINGGGSFAAYGDAVKGRFTISRDNA |
| | | KNTLYLQMNSLRAEDTAMYYCVGGGYMDFWGPGTSLFVSSASE |
| | | QKLISEEDLPSGGGGSGGGGSSPTEPSSKTGNPRHLHVLIGTSVVK |
| | | IPFTILLFFLLHRWCSNKKNAAVMDQEPAGNRTVNSEDSDEQDH |
| | | QEVSYA |
| 84 | scFv_42 | MALPVTALLLPLALLLHAARPGSQSVLTQPTSVSGSLGQRVTISCS |
| | CAR | GSTNDIGIIGAAWYQQLPGKAPKLLLYSNKNRPSGVPDRFSGSNS |
| | | GISATLTITGLQAEDEADYYCQSVDPALHTYVFGSGTQLTILGGGS |
| | | SRSSSSGGGGSGGGGEVQLVEIGGDLVKPEGSLRLSCVVSGFTFS |
| | | GYDMNWVRQAPGKGLQWVAYISSDGRRTVYTDAVKGRFTISRD |
| | | NAKNTLYLQMNSLKSEDTAVYYCVKGGWLDFWGQGTLVTVSS |
| | | ASEQKLISEEDLPSGGGGSGGGGSSPTEPSSKTGNPRHLHVLIGTS |
| | | VVKIPFTILLFFLLHRWCSNKKNAAVMDQEPAGNRTVNSEDSDE |
| | | QDHQEVSYA |
| 85 | scFv_43 | MALPVTALLLPLALLLHAARPGSDIVMTQNPLSLAVTPGELATISC |
| | CAR | RASQSLLHSDGKSYLNWYLQKPGQTPRPLIYEASRRFSGVSDRFN |
| | | GSGSGTDFTLKISRVEAEDVGVYYCQQGLHFPPTFGAGTKVELKG |
| | | GSSRSSSSGGGGSGGGGEVQLVESGGDLVKPGGSLRLSCAASGFT |
| | | FTDYDMSWVRQAPGKGLQWVAAISYDGSSTYYTDAVKGRFTISR |
| | | DNARNTLYLQMSSLRAEDTAMYYCVVSNMNLWGHGTSVFVSSA |
| | | SEQKLISEEDLPSGGGGSGGGGSSPTEPSSKTGNPRHLHVLIGTSV |
| | | VKIPFTILLFFLLHRWCSNKKNAAVMDQEPAGNRTVNSEDSDEQ |
| | | DHQEVSYA |

### Transmembrane Domain

With respect to the transmembrane domain, the CAR of the present invention can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e., comprise at least the transmembrane region(s) of) the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present invention includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

The hinge region can have a length of from about 4 amino acids to about 50 amino acids, e.g., from about 4 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 40 amino acids, or from about 40 amino acids to about 50 amino acids.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, hinge regions include glycine polymers (G)ₙ, glycine-serine polymers (including, for example, (GS)ₙ, (GSGGS), (SEQ ID NO: 11) and (GGGS)ₙ (SEQ ID NO: 13), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO: 18), GGSGG (SEQ ID NO: 19), GSGSG (SEQ ID NO: 20), GSGGG (SEQ ID NO: 21), GGGSG (SEQ ID NO: 22), GSSSG (SEQ ID NO: 23), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1): 162-166; and Huck et al., Nucleic Acids Res. (1986) 14(4): 1779-1789. As non-limiting examples, an immunoglobulin hinge region can include one of the following amino acid sequences: DKTHT (SEQ ID NO: 24); CPPC (SEQ ID NO: 25); CPEPKSCDTPPPCPR (SEQ ID NO: 26) (see, e.g., Glaser et al., J. Biol. Chem. (2005) 280:41494-41503); ELKTPLGDTTHT (SEQ ID NO: 27); KSCDKTHTCP (SEQ ID NO: 28); KCCVDCP (SEQ ID NO: 29); KYGPPCP (SEQ ID NO: 30); EPKSCDKTHTCPPCP (SEQ ID NO: 31) (human IgG1 hinge); ERKCCVECPPCP (SEQ ID NO: 32) (human IgG2 hinge); ELKTPLGDTTHTCPRCP (SEQ ID NO: 33) (human IgG3 hinge); SPNMVPHAHHAQ (SEQ ID NO: 34) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally occurring) hinge region. For example, His229 of human IgG1 hinge can be substituted with Tyr, so that the hinge region comprises the sequence EPKSCDKTYTCPPCP (SEQ ID NO: 31); see, e.g., Yan et al., J. Biol. Chem. (2012) 287: 5891-5897. In one embodiment, the hinge region can comprise an amino acid sequence derived from human CD8, or a variant thereof.

The transmembrane domains described herein, such as a transmembrane region of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9, can be combined with any of the antigen binding domains described herein, any of the costimulatory signaling domains or intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. In exemplary embodiments, a triplet of phenylalanine, tryptophan and valine will be found at each end of a synthetic transmembrane domain.

In some embodiments, a subject CAR may further comprise, between the extracellular domain and the transmembrane domain of the CAR, or between the intracellular domain and the transmembrane domain of the CAR, a spacer domain. As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

Accordingly, a subject CAR of the present invention may comprise any of the transmembrane domains, hinge domains, or spacer domains described herein.

### Intracellular Domain

A subject CAR of the present invention also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a KIR2DS2 domain. In certain embodiments, the intracellular domain comprises a costimulatory signaling domain and an intracellular signaling domain.

In one embodiment, the intracellular domain of the CAR comprises a costimulatory signaling domain which includes any portion of one or more co-stimulatory molecules, such as at least one signaling domain from CD2, CD3, CD8, CD27, CD28, OX40, ICOS, 4-1BB, PD-1, any derivative or variant thereof, any synthetic sequence thereof that has the same functional capability, and any combination thereof.

Examples of the intracellular signaling domain include, without limitation, the ζ chain of the T cell receptor complex or any of its homologs, e.g., η chain, FesRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD lib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent).

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR.

### NK Cell Receptor CARs

In some aspects, the subject CAR of the present invention provides a CAR which shares functional and structural properties with a NK cell immune-function receptor (or NKR). In certain embodiments of the present invention, a variety of NKRs can serve as the basis for an NKR-CAR.

### NK Cell Immune-Function Receptors (NKRS) and NK Cells

As disclosed herein, NK cell immune-function receptor (or NKR) refers to an endogenous naturally occurring transmembrane protein expressed in NK cells, which can engage with a ligand on an antigen presenting cell and modulate an NK cell immune-function response, e.g., the cytolytic activity or cytokine secretion of the NK cell. NK cells are mononuclear cells that develop in the bone marrow from lymphoid progenitors and are typically identified by the expression of the cluster determinants (CDs) CD 16, CD56, and/or CD57, as well as the absence of the alpha/beta or gamma/delta TCR complex on the cell surface. Functionally, NK cells possess the ability to bind to and kill target cells that fail to express “self” major histocompatibility complex (MHC)/human leukocyte antigen (HLA) proteins; and the ability to kill tumor cells or other diseased cells that express ligands for activating NK receptors. NK cells are characterized by their ability to bind and kill 5 several types of tumor cell lines without the need for prior immunization or activation. NK cells can also release soluble proteins and cytokines that exert a regulatory effect on the immune system; and can undergo multiple rounds of cell division and produce daughter cells with similar biologic properties as the parent cell. Upon activation by interferons and/or cytokines, NK cells mediate the lysis of tumor cells and of cells infected with intracellular pathogens by mechanisms that require direct, physical contacts between the NK cell and the target cell. Lysis of target cells involves the release of cytotoxic granules from the NK cell onto the surface of the bound target, and effector proteins such as perforin and granzyme B that penetrate the target plasma membrane and induce apoptosis or programmed cell death. Normal, healthy cells are protected from lysis by NK cells. NK cell activity is regulated by a complex mechanism that involves both stimulating and inhibitory signals.

Briefly, the lytic activity of NK cells is regulated by various cell surface receptors that transduce either positive or negative intracellular signals upon interaction with ligands on the target cell. The balance between positive and negative signals transmitted via these receptors determine whether or not a target cell is lysed (killed) by a NK cell. NK cell stimulatory signals can be mediated by Natural Cytotoxicity Receptors (NCR) such as NKp30, NKp44, and NKp46; as well as NKG2C receptors, NKG2D receptors, certain activating killer cell immunoglobulin-like receptors (KIRs), and other activating NK receptors (Lanier, Annual Review of Immunology 2005; 23:225-74). NK cell inhibitory signals can be mediated by receptors like Ly49, CD94/NKG2A, as well as certain inhibitory KIRs, which recognize major histocompatibility complex (MHC) class I molecules (Karre et al., Nature 1986; 319:675-8; Ohlen et al, Science 1989; 246:666-8). These inhibitory receptors bind to polymorphic determinants of MHC class I molecules (including HLA class I) present on other cells and inhibit NK cell-mediated lysis.

### KIR-CARS

In certain embodiments of the present invention, a subject CAR comprises an antigen binding domain and a killer cell immunoglobulin-like receptor domain (KIR-CAR). In certain embodiments, the KIR-CAR of the invention is expressed on the surface of an immune effector cell, e.g., a T cell or a NK cell.

KIRs, referred to as killer cell immunoglobulin-like receptors, have been characterized in humans and non-human primates, and are polymorphic type 1 trans-membrane molecules present on certain subsets of lymphocytes, including NK cells and some T cells. KIRs interact with determinants in the alpha 1 and 2 domains of the MHC class I molecules and, as described elsewhere herein, distinct KIRs are either stimulatory or inhibitory for NK cells.

NKR-CARs described herein include KIR-CARs, which share functional and structural properties with KIRs.

KIRs are a family of cell surface proteins found on NK cells, which regulate the killing function of these cells by interacting with MHC class I molecules, which are expressed on all cell types. This interaction allows them to detect virally infected cells or tumor cells. Most KIRs are inhibitory, meaning that their recognition of MHC suppresses the cytotoxic activity of the NK cell that expresses them. Only a limited number of KIRs have the ability to activate cells. The KIR gene family has at least 15 gene loci (KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/SI, KIR3DL2, KIR3DL3, and two pseudogenes, KIR2DP1 and KIR3DP1) encoded within a 100-200 Kb region of the Leukocyte Receptor Complex (LRC) located on chromosome 19 (19q13.4). The LRC constitutes a large, 1 Mb, and dense cluster of rapidly evolving immune genes which contains genes encoding other cell surface molecules with distinctive lg-like extra-cellular domains. In addition, the extended LRC contains genes encoding the trans membrane adaptor molecules DAP10 and DAP12.

KIR genes vary in length from 4 to 16 Kb (full genomic sequence) and can contain four to nine exons. KIR genes are classified as belonging to one of three groups according to their structural features: (1) Type I KIR2D genes, which encode two extra-cellular domain proteins with a D1 and D2 conformation; (2) The structurally divergent Type II KIR2D genes which encode two extra-cellular domain proteins with a DO and D2 conformation; and finally (3) KIR3D genes encoding proteins with three extra-cellular lg-like domains (DO, D1 and D2). Type I KIR2D genes, which include the pseudogene KIR2DP1 as well as KIR2DL1-3 and KIR2DS 1-5 genes, possess eight exons as well as a pseudoexon 3 sequence. This pseudoexon is inactivated in Type I KIR2D. In some cases, this is due to a nucleotide substitution located on the intron 2-exon 3 splice-site where its nucleotide sequence exhibits a high-degree of identity to KIR3D exon 3 sequences and possesses a characteristic three base pair deletion. In other cases, a premature stop codon initiates differential splicing of exon 3. Within the Type I KIR2D group of genes, KIR2DL1 and KIR2DL2 share a common deletion in exon 7 distinguishing them from all other KIR in this exon, which results in a shorter exon 7 sequence. Similarly, within Type I KIR2D, KIR2DL1-3 differ from KIR2DS1-5 only in the length of their cytoplasmic tail encoding region in exon 9. The KIR2DP1 pseudogene structure differs from that of KIR2DL1-3 in that the former has a shorter exon 4 sequence, due to a single base pair deletion.

Type II KIR2D genes include KIR2DL4 and KIR2DL5. Unlike KIR3D and Type I KIR2D, Type II KIR2D characteristically have deleted the region corresponding to exon 4 in all other KIR. Additionally, Type II KIR2D genes differ from Type I KIR2D genes in that the former possess a translated exon 3, while Type I KIR2D genes have an untranslated pseudoexon 3 sequence in its place. Within the Type II KIR2D genes, KIR2DL4 is further differentiated from KIR2DL5 (as well as from other KIR genes) by the length of its exon 1 sequence. In KIR2DL4, exon 1 was found to be six nucleotides longer and to possess an initiation codon different from those present in the other KIR genes. This initiation codon is in better agreement with the Kozak transcription initiation consensus sequence′ than the second potential initiation codon in KIR2DL4 that corresponds to the initiation codon present in other KIR genes.

KIR3D genes possess nine exons and include the structurally related KIR3DL1, KIR3DS1, KIR3DL2 and KIR3DL3 genes. KIR3DL2 nucleotide sequences are the longest of all KIR genes and span 16,256 bp in full genomic sequences and 1,368 bp in cDNA. Within the KIR3D group, the four KIR genes differ in the length of the region encoding the cytoplasmic tail in exon 9. The length of the cytoplasmic tail of KIR proteins can vary from 14 amino acid residues long (in some KIR3DS 1 alleles) to 108 amino acid residues long (in KIR2DL4 proteins). Additionally, KIR3DS1 differs from KIR3DL1 or KIR3DL2 in that the former has a shorter exon 8 sequence. KIR3DL3 differs from other KIR sequences in that it completely lacks exon 6. The most extreme KIR gene structure difference observed was that of KIR3DP1. This gene fragment completely lacks exons 6 through 9, and occasionally also exon 2. The remaining portions of the gene which are present (exon 1, 3, 4 and 5) share a high level of sequence identity to other KIR3D sequences, in particular to KIR3DL3 sequences.

KIR proteins possess characteristic lg-like domains on their extracellular regions, which in some KIR proteins are involved in HLA class I ligand binding. They also possess transmembrane and cytoplasmic regions which are functionally relevant as they define the type of signal which is transduced to the NK cell. KIR proteins can have two or three lg-like domains (hence KIR2D or KIR3D) as well as short or long cytoplasmic tails (represented as KIR2DS or KIR2DL). Two domain KIR proteins are subdivided into two groups depending on the origin of the membrane distal lg-like domains present. Type I KIR2D proteins (KIR2DL1, KIR2DL2, KIR2DL3, KIR2DS1, KIR2DS2, KIR2DS3, KIR2DS4 and KIR2DS5) possess a membrane-distal lg-like domain similar in origin to the KIR3D D1 lg-like domain but lack a DO domain. This D1 lg-like domain is encoded mainly by the fourth exon of the corresponding KIR genes. The Type II KIR2D proteins, KIR2DL4 and KIR2DL5, possess a membrane-distal lg-like domain of similar sequence to the DO domain present in KIR3D proteins, however, Type II KIR2D lack a D1 domain. Long cytoplasmic tails usually contain two Immune Tyrosine-based Inhibitory Motifs (ITIM) which transduce inhibitory signals to the NK cell. Short cytoplasmic tails possess a positively charged amino acid residue in their transmembrane region which allows them to associate with a DAP12 signaling molecule capable of generating an activation signal Exceptions to this is KIR2DL4, which contains only one N-terminus ITIM. In addition, KIR2DL4 also possesses a charged residue (arginine) in its transmembrane domain, a feature which allows this receptor to elicit both inhibitory and activating signals. KIR control the response of human NK cells by delivering inhibitory or activating signals upon recognition of MHC class I ligands on the surface of potential target cells.

KIR proteins vary in length from 306 to 456 amino acid residues. Although the differences in protein length are mostly the consequence of the number of lg-like domains present, cytoplasmic region length diversity is also an influencing factor. The leader peptide of most KIR proteins is 21 amino acid residues long. However, the presence of a different initiation codon generates a correspondingly longer leader peptide in KIR2DL4 proteins. The DO lg-like domain present in Type II KIR2D proteins and KIR3D proteins is approximately 96 amino acid residues in length. The D1 domain of Type I KIR2D and of KIR3D proteins is 102 amino acid residues long, while the D2 domain of all KIR proteins is 98 amino acid residues long. The length of the stem region varies from the 24 amino acid residues present in most KIR proteins, to only seven amino acid residues in the divergent KIR3DL3 protein. The transmembrane region is 20 amino acid residues long for most proteins, but one residue shorter on KIR2DL1 and KIR2DL2 proteins as a result of a three base pair deletion in exon 7. Finally, the cytoplasmic region of KIR proteins exhibits greater length variations, ranging from 23 amino acid residues in some KIR3DS 1 alleles to the 96 amino acid residues present in KIR3DL2 proteins.

Amino acid sequences for human KIR polypeptides (Homo sapiens) are available in the KIR NCBI database, see e.g., accession number NP_037421.2 (GI: 134268644), NP_703144.2 (GI: 46488946), NP_001229796.1 (GI: 338968852), NP_001229796.1 (GI: 338968852), NP 006728.2 (GI: 134268642), NP_065396.1 (GI: 11968154), NP_001018091.1 (GI: 66267727), NP_001077008.1 (GI: 134133244), NP_036444.1 (GI: 6912472), NP_055327.1 (GI: 7657277), NP_056952.2 (GI: 71143139), NP_036446.3 (GI: 116517309), NP_001074239.1 (GI: 124107610), NP_002246.5 (GI: 124107606), NP_001074241.1 (GI: 124107604), NP_036445.1 (GI: 6912474).

The nomenclature for KIRs is based upon the number of extracellular domains (KIR2D and KIR3D having two and three extracellular lg-domains, respectively) and whether the cytoplasmic tail is long (KIR2DL or KIR3DL) or short (KIR2DS or KIR3DS). The presence or KIR absence of a given is variable from one NK cell to another within the NK population present in a single individual. Among humans, there is also a relatively high level of polymorphism of KIR genes, with certain KIR genes being present in some, but not all individuals. The expression of KIR alleles on NK cells is stochastically regulated, meaning that, in a given individual, a given lymphocyte may express one, two, or more different KIRs, depending on the genotype of the individual. The NK cells of a single individual typically express different combinations of KIRs, providing a repertoire of NK cells with different specificities for MHC class I molecules. Certain KIR gene products cause stimulation of lymphocyte activity when bound to an appropriate ligand. The activating KIRs all have a short cytoplasmic tail with a charged trans-membrane residue that associates with an adapter molecule having an Immunoreceptor Tyrosine-based Activation Motifs (ITAMs) which transduce stimulatory signals to the NK cell. By contrast, inhibitory KIRs have a long cytoplasmic tail containing Immunoreceptor Tyrosine-based Inhibitory Motif (ITIM), which transduce inhibitory signals to the NK cell upon engagement of their MHC class I ligands. The known inhibitory KIRs include members of the KIR2DL and KIR3DL subfamilies. Inhibitory KIRs having two lg domains (KIR2DL) recognize HLA-C allotypes: KIR2DL2 (formerly designated p58.2) and the closely related, allelic gene product KIR2DL3 both recognize “group 1” HLA-C allotypes (including HLA-Cw1, -3, -7, and -8), whereas KIR2DL1 (p58.1) recognizes “group 2” HLA-C allotypes (such as HLA-Cw2, -4, -5, and -6). The recognition by KIR2DL1 is dictated by the presence of a Lys residue at position 80 of HLA-C alleles. KIR2DL2 and KIR2DL3 recognition is dictated by the presence of an Asn residue at position 80 in HLA-C. Importantly, the great majority of HLA-C alleles have either an Asn or a Lys residue at position 80. Therefore, KIR2DL1, -2, and -3 collectively recognize essentially all HLA-C allotypes found in humans. One KIR with three lg domains, KIR3DL1 (p70), recognizes an epitope shared by HLA-Bw4 alleles. Finally, KIR3DL2 (p140), a homodimer of molecules with three lg domains, recognizes HLA-A3 and -HLA-A11.

However, the invention should not be limited to inhibitory KIRs comprising a cytoplasmic tail containing ITIM. Rather, any inhibitory protein having a cytoplasmic domain that is associated with an inhibitory signal can be used in the construction of the CARs of the invention. Non-limiting examples of an inhibitory protein include but are not limited CTLA-4, PD-1, and the like. These proteins are known to inhibit T cell activation.

Accordingly, in some aspects, a subject CAR of the present invention comprises a KIR-CAR comprising an extracellular domain that comprises a target-specific or antigen-specific binding domain, fused to a KIR or fragment thereof. In one embodiment, the KIR is an activating KIR that comprises a short cytoplasmic tail that associates with an adapter molecule having an Immunoreceptor Tyrosine-based Activation Motifs (ITAMs) which transduce stimulatory signals to the NK cell (referred elsewhere herein as actKIR-CAR). In certain embodiments, the KIR is an inhibitory KIR that comprises a long cytoplasmic tail containing Immunoreceptor Tyrosine based Inhibitory Motif (ITIM), which transduces inhibitory signals (referred to herein as inhKIR-CAR). In certain embodiments, it is desirable to remove the hinge region for the activating KIRs when construction an actKIR-CAR. In certain embodiments, an activating KIR CAR in which the KIR2DS2 hinge is removed to generate a KIR2S CAR, which KIRS2 CAR exhibits enhanced cytolytic activity compared to an actKIR-CAR comprising a full-length wildtype KIR2DS2.

KIR hinge or stem domain, as that term is used herein, refers to a polypeptide domain having structural and functional properties of a hinge or stem domain of a KIR. In some embodiments the KIR hinge or stem domain of a KIR-CAR has at least 70, 80, 85, 90, 95, or 99% homology with a reference sequence, e.g., a naturally occurring KIR hinge or stem domain or a KIR hinge or stem domain described herein. In some embodiments the KIR hinge or stem domain of a KIR-CAR differs at no more than 15, 10, 5, 2, or 1% of its residues from a reference sequence, e.g., a naturally occurring KIR hinge or stem domain or a KIR hinge or stem domain described herein. In some embodiments the KIR hinge or stem domain of a KIR-CAR differs at no more than 5, 4, 3, 2 or 1 residue from a reference sequence, e.g., a naturally occurring KIR hinge or stem domain or a KIR hinge or stem domain described herein. In some embodiments the KIR hinge or stem domain of a KIR-CAR does not differ from, or shares 100% homology with, a reference sequence, e.g., a naturally occurring KIR hinge or stem domain or a KIR hinge or stem domain described herein.

### NCRs

NKR-CARs described herein include NCR-CARs, which share functional and structural properties with NCRs.

Natural killer (NK) cells are cytotoxic lymphoid cells specialized in destroying tumors and virus-infected cells. Unlike cytotoxic T lymphocytes, NK cells do not express antigen-specific receptors. The recognition of transformed cells occurs via the association of a multitude of cell-surface receptors with surface markers on the target cell. The NK cell surface receptors can be distinguished according to whether they activate or inhibit NK cell-mediated cytotoxicity. Numerous interactions between different receptors appear to lead to the formation of synapses between NK and target cells. The integration of activating and inhibiting signals at the synapse dictates whether or not the NK cells exert their cytolytic function on the target cell. Among the activating receptors, the family of lg-like molecules is termed natural cytotoxicity receptors (NCRs). These natural cytotoxicity receptors include NKp30, NKp44 and NKp46 molecules. The NCRs are key activating receptors for NK cells in tumor cell recognition. All three NCRs are involved in the clearance of both tumor and virus-infected cells. In the latter, the antiviral activity is initiated by the interaction of NKp44 with hemagglutinin of influenza virus or Sendai virus. NKp46 targets virus-infected cells by binding to influenza virus hemagglutinin or Sendai virus hemagglutinin-neuraminidase. In contrast, it has been shown that NK cell-mediated cytotoxicity is inhibited by binding of NKp30 to the human cytomegaloviral protein pp65 (see, e.g., Amon, et. al., Nat. Immunol. (2005) 6:515-523).

Amino acid sequences for human NCR polypeptides (Homo sapiens) are available in the NCBI database, see e.g., accession number NP_004819.2 (GI: 153945782), 014931.1 (GI: 47605770), 095944.2 (GI: 251757303), 076036.1 (GI: 47605775), NP_001138939.1 (GI: 224586865), and/or NP_001138938.1 (GI: 224586860).

In an embodiment, an NCR-CAR comprises an antigen binding domain and an NCR transmembrane domain. In some embodiments, a KIR-CAR comprises an antigen binding domain and an NCR intracellular domain.

NCR extracellular domain, as that term is used herein, refers to a polypeptide domain having structural and functional properties of an extracellular domain of an NCR. In an embodiment the NCR extracellular domain of an NCR-CAR has at least 70, 80, 85, 90, 95, or 99% homology with a reference sequence, e.g., a naturally occurring NCR extracellular domain or an NCR extracellular domain described herein. In some embodiments the NCR extracellular domain of an NCR-CAR differs at no more than 15, 10, 5, 2, or 1% of its residues from a reference sequence, e.g., a naturally occurring NCR extracellular domain or an NCR extracellular domain described herein. In some embodiments the NCR extracellular domain of an NCR-CAR differs at no more than 5, 4, 3, 2 or 1 residue from a reference sequence, e.g., a naturally occurring NCR extracellular domain or an NCR extracellular domain described herein. In some embodiments the NCR extracellular domain of an NCR-CAR does not differ from, or shares 100% homology with, a reference sequence, e.g., a naturally occurring NCR extracellular domain or an NCR extracellular domain described herein.

NCR hinge or stem domain, as that term is used herein, refers to a polypeptide domain having structural and functional properties of a hinge or stem domain of an NCR. In an embodiment the NCR hinge or stem domain of an NCR-CAR has at least 70, 80, 85, 90, 95, or 99% homology with a reference sequence, e.g., a naturally occurring NCR hinge or stem domain or an NCR hinge or stem domain described herein. In some embodiments the NCR hinge or stem domain of an NCR-CAR differs at no more than 15, 10, 5, 2, or 1% of its residues from a reference sequence, e.g., a naturally occurring NCR hinge or stem domain or an NCR hinge or stem domain described herein. In some embodiments the NCR hinge or stem domain of an NCR-CAR differs at no more than 5, 4, 3, 2 or 1 residue from a reference sequence, e.g., a naturally occurring NCR hinge or stem domain or an NCR hinge or stem domain described herein. In embodiments the NCR hinge or stem domain of an NCR-CAR does not differ from, or shares 100% homology with a reference sequence, e.g., a naturally occurring NCR hinge or stem domain or an NCR hinge or stem domain described herein.

NCR transmembrane domain, as that term is used herein, refers to a polypeptide domain having structural and functional properties of a transmembrane domain of an NCR. In an embodiment the NCR transmembrane domain of an NCR-CAR has at least 70, 80, 85, 90, 95, or 99% homology with a reference sequence, e.g., a naturally occurring NCR transmembrane domain or an NCR transmembrane domain described herein. In some embodiments the NCR transmembrane domain of an NCR-CAR differs at no more than 15, 10, 5, 2, or 1% of its residues from a reference sequence, e.g., a naturally occurring NCR transmembrane domain or an NCR transmembrane domain described herein. In some embodiments the NCR transmembrane domain of an NCR-CAR differs at no more than 5, 4, 3, 2 or 1 residue from a reference sequence, e.g., a naturally occurring NCR transmembrane domain or an NCR transmembrane domain described herein. In some embodiments the NCR transmembrane domain of an NCR-CAR does not differ from, or shares 100% homology with, a reference sequence, e.g., a naturally occurring NCR transmembrane domain or an NCR transmembrane domain described herein.

NCR intracellular domain, as that term is used herein, refers to a polypeptide domain having structural and functional properties of an intracellular domain of an NCR. In some embodiments the NCR intracellular domain of an NCR-CAR has at least 70, 80, 85, 90, 95, or 99% homology with a reference sequence, e.g., a naturally occurring NCR intracellular domain or an NCR intracellular domain described herein. In some embodiments the NCR intracellular domain of an NCR-CAR differs at no more than 15, 10, 5, 2, or 1% of its residues from a reference sequence, e.g., a naturally occurring NCR intracellular domain or an NCR intracellular domain described herein. In some embodiments the NCR intracellular domain of an NCR-CAR differs at no more than 5, 4, 3, 2 or 1 residue from a reference sequence, e.g., a naturally occurring NCR intracellular domain or an NCR intracellular domain described herein. In some embodiments the NCR intracellular domain of an NCR-CAR does not differ from, or shares 100% homology with, a reference sequence, e.g., a naturally occurring NCR intracellular domain or an NCR intracellular domain described herein.

### Modified Immune Cells

The present invention provides a modified immune cell or precursor cell thereof (e.g., a modified T cell, a modified NK cell, a modified NKT cell, a modified macrophage), comprising a fusion polypeptide comprising a subject CAR. Accordingly, such modified cells possess the specificity directed by the CAR that is expressed therein. For example, a modified cell of the present invention comprising a hCD19 CAR possesses specificity for hCD19 on a target cell or a mesothelin CAR possesses specificity for mesothelin on a target cell. The fusion polypeptides further comprise second polypeptide domains (e.g., a Rab5 or Rab11 protein) connected to the CAR via a cleavable linker such that expression of the CAR results in expression of the Rab5 or Rab11 protein. In some embodiments, the co-expression of the Rab5 or Rab11 proteins assists in maintaining the persistence or expression of the CAR at the surface of the modified immune cell or precursor thereof, such that function of the cell is maintained or improved.

Any modified cell comprising a fusion polypeptide comprising a Rab5 or Rab11 polypeptide domain cleavable linked to a CAR comprising any antigen binding domain, any hinge, any transmembrane domain, any intracellular costimulatory domain, and any intracellular signaling domain described herein is envisioned, and can readily be understood and made by a person of skill in the art in view of the disclosure herein.

In some embodiments, the modified cell is an immune cell or precursor cell thereof. In an exemplary embodiment, the modified cell is a T cell. In an exemplary embodiment, the modified cell is an autologous cell. In an exemplary embodiment, the modified cell is an autologous immune cell or precursor cell thereof. In an exemplary embodiment, the modified cell is an autologous T cell.

### Fusion Polypeptides, Nucleic Acids, and Expression Vectors

The present invention provides an isolated nucleic acid encoding a fusion polypeptide. The nucleic acids of the present invention can comprise a polynucleotide sequence encoding any one of the binding fusion polypeptides, scFvs, CARs, or any fragments thereof disclosed herein.

One aspect of the present invention includes a fusion polypeptide comprising a CAR and a Rab5 or Rab11 polypeptide or a Rab5 and Rab11 polypeptide. In certain embodiments, the fusion polypeptide comprises a CAR that specifically binds human CD19. In certain embodiments, the fusion polypeptide comprises a CAR that specifically binds to mesothelin.

In certain embodiments, the fusion polypeptide comprises an amino acid sequence set forth in SEQ ID NOs: 1-2 or 43-85.

Tolerable variations of the fusion polypeptide will be known to those of skill in the art, while maintaining specific binding to the tumor antigen and providing functional Rab5 or Rab11 proteins. For example, in some embodiments the fusion polypeptide comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 1-2, or 43-85.

In some embodiments, the fusion polypeptide comprising the CAR and Rab5 or Rab11 protein is encoded by a nucleic acid sequence encoding the fusion polypeptide comprising the CAR and Rab5 or Rab11 protein.

Another aspect of the present invention provides a vector comprising any one of the isolated nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

Also provided is a host cell comprising any of the vectors or nucleic acids disclosed herein. The host cell can be of eukaryotic, prokaryotic, mammalian, or bacterial origin. Non-limiting examples of cells that can be used to express the fusion polypeptides disclosed herein include Human embryonic kidney (HEK) cell lines (e.g., HEK293), Chinese hamster ovary (CHO) cell lines, Baby hamster kidney (BHK) cell lines, COS cell lines, Madin Darby canine kidney (MDCK) cell line, and HeLa cell lines. In some cases, the host cell is a Chinese Hamster Ovary cell.

A method of producing a fusion polypeptide that comprises a CAR and a Rab5 or Rab11 protein is also provided herein, wherein the method comprises culturing the host cell. In some embodiments, the method further comprises incubating the host cell in a cell culture medium under conditions sufficient to allow expression of the nucleic acid encoding the fusion polypeptide described herein.

In some embodiments, a nucleic acid of the present invention can be operably linked to a transcriptional control element, e.g., a promoter, and enhancer, etc. Suitable promoter and enhancer elements are known to those of skill in the art.

In certain embodiments, the nucleic acid is in operable linkage with a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, a spv promoter, and the like; a promoter derived from the pathogenicity island SPI-2 (see, e.g., WO96/17951); an actA promoter (see, e.g., Shetron-Rama et al., Infect. Immun. (2002) 70:1087-1096); a rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the present invention should not be limited to the use of constitutive promoters. Inducible promoters are also contemplated as part of the present invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch can make use of a Cre-lox-mediated recombination (see, e.g., Fuhrmann-Benzakein, et al., Proc. Natl. Acad. Sci. USA (2000) 28: e99, the present invention of which is incorporated herein by reference). Any suitable combination of recombinase, endonuclease, ligase, recombination sites, etc. known to the art can be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the present disclosures of which are incorporated herein by reference.

A nucleic acid of the present invention can be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host can be present. Suitable expression vectors include, but are not limited to, viral vectors (e.g., viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4: 683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) can be used to introduce the nucleic acid into a host cell. Accordingly, an expression vector (e.g., a lentiviral vector) of the present invention can comprise a nucleic acid encoding a polypeptide. In some embodiments, the expression vector (e.g., lentiviral vector) will comprise additional elements that will aid in the functional expression of the polypeptide encoded therein. In some embodiments, an expression vector comprising a nucleic acid encoding for a polypeptide further comprises a mammalian promoter. In one embodiment, the vector further comprises an elongation-factor-1-alpha promoter (EF-1α promoter). Use of an EF-1α promoter can increase the efficiency in expression of downstream transgenes. Physiologic promoters (e.g., an EF-1α promoter) can be less likely to induce integration mediated genotoxicity and can abrogate the ability of the retroviral vector to transform stem cells. Other physiological promoters suitable for use in a vector (e.g., lentiviral vector) are known to those of skill in the art and can be incorporated into a vector of the present invention. In some embodiments, the vector (e.g., lentiviral vector) further comprises a non-requisite cis acting sequence that can improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and can be incorporated into a vector (e.g., lentiviral vector) of the present invention. In some embodiments, the vector further comprises a posttranscriptional regulatory element. Posttranscriptional regulatory elements can improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and can be incorporated into a vector (e.g., lentiviral vector) of the present invention. A vector of the present invention can further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention can comprise any combination of the elements described herein to enhance the efficiency of functional expression of transgenes. For example, a vector (e.g., lentiviral vector) of the present invention can comprise a WPRE sequence, cPPT sequence, RRE sequence, 5′LTR, 3′ U3 deleted LTR′ in addition to a nucleic acid encoding for a CAR.

Vectors of the present invention can be self-inactivating vectors. As used herein, the term “self-inactivating vector” refers to vectors in which the 3′ LTR enhancer promoter region (U3 region) has been modified (e.g., by deletion or substitution). A self-inactivating vector can prevent viral transcription beyond the first round of viral replication. Consequently, a self-inactivating vector can be capable of infecting and then integrating into a host genome (e.g., a mammalian genome) only once, and cannot be passed further. Accordingly, self-inactivating vectors can greatly reduce the risk of creating a replication-competent virus.

In some embodiments, a nucleic acid of the present invention can be RNA, e.g., in vitro synthesized RNA. Methods for in vitro synthesis of RNA are known to those of skill in the art; any known method can be used to synthesize RNA comprising a sequence encoding a polypeptide of the present invention. Methods for introducing RNA into a host cell are known in the art. See, e.g., Zhao et al. Cancer Res. (2010) 15: 9053. Introducing RNA comprising a nucleotide sequence encoding a polypeptide of the present invention into a host cell can be carried out in vitro, ex vivo or in vivo. For example, a host cell (e.g., an NK cell, a cytotoxic T lymphocyte, a macrophage etc.) can be electroporated in vitro or ex vivo with RNA comprising a nucleotide sequence encoding a polypeptide of the present invention.

In order to assess the expression of a polypeptide or portions thereof, the expression vector to be introduced into a cell can also contain either a selectable marker gene or a reporter gene, or both, to facilitate identification and selection of expressing cells from the population of cells sought to be transfected or infected through viral vectors. In some embodiments, the selectable marker can be carried on a separate piece of DNA and used in a co-transfection procedure. Both selectable markers and reporter genes can be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes can include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present invention is provided for the production of a polypeptide as described herein, e.g., in a host cell. In some embodiments, a nucleic acid of the present invention provides for amplification of the polypeptide-encoding nucleic acid.

### Methods of Generating Modified Immune Cells

The present invention provides methods for producing/generating a modified immune cell or precursor cell thereof (e.g., a T cell/NK cell/NKT cell/macrophage). The cells are generally engineered by introducing a nucleic acid encoding a subject fusion polypeptide comprising a CAR (e.g., hCD19 CAR) and a second polypeptide (e.g., a Rab5 protein or a Rab11 protein).

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, MA) or the Gene Pulser II (BioRad, Denver, CO), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8): 861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a subject CAR of the invention is introduced into a cell by an expression vector. Expression vectors comprising a nucleic acid encoding a subject CAR (e.g., CD19 CAR) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding a subject CAR) may be inserted to substitute large pieces of adenoviral DNA in order to make the expression vector of the present invention (see, e.g., Danthinne and Imperiale, Gene Therapy (2000) 7(20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR) into the viral genome at certain locations to produce a virus that is replication defective. Though the retrovirus vectors are able to infect a broad variety of cell types, integration and stable expression of the subject CAR, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the human immunodeficiency viruses (HIV-1, HIV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR (see, e.g., U.S. Pat. No. 5,994,136).

Expression vectors including a nucleic acid of the present invention can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, an NKT cell, or a macrophage.

The present invention also provides genetically engineered cells which include and stably express a subject CAR of the present invention. In some embodiments, the genetically engineered cells are genetically engineered T-lymphocytes (T cells), regulatory T cells (Tregs), naive T cells (TN), memory T cells (for example, central memory T cells (TCM), effector memory cells (TEM)), natural killer cells (NK cells), natural killer T cells (NKT cells) and macrophages capable of giving rise to therapeutically relevant progeny. In one embodiment, the genetically engineered cells are autologous cells.

Modified cells (e.g., comprising a subject CAR) may be produced by stably transfecting host cells with an expression vector including a nucleic acid of the present invention. Additional methods to generate a modified cell of the present invention include, without limitation, chemical transformation methods (e.g., using calcium phosphate, dendrimers, liposomes and/or cationic polymers), non-chemical transformation methods (e.g., electroporation, optical transformation, gene electro transfer and/or hydrodynamic delivery) and/or particle-based methods (e.g., impalefection, using a gene gun and/or magnetofection). Transfected cells expressing a subject CAR of the present invention may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about-20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

### Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells and/or NKT cells and/or macrophages. Other exemplary cells include stem cells, such as multipotent and pluripotent stem cells, including induced pluripotent stem cells (iPSCs). In certain aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the cells are derived or isolated is blood or a blood-derived sample or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity-based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed, and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed, and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In certain exemplary embodiments, multiple rounds of separation steps are carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker-) or express relatively low levels (marker^{low}) of one or more markers. For example, in certain aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In certain exemplary embodiments, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations. In some embodiments, combining TCM-enriched CD8+ T cells and CD4+ T cells further enhance efficacy.

In some embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and/or a CD8+ T population is enriched for central memory (TCM) cells. In some embodiments, the enrichment for central memory T (TCM) cells is based on positive or high surface expression of CD45RO, CD62L, CCR7, CD28, CD3, and/or CD 127; in certain aspects, it is based on negative selection for cells expressing or highly expressing CD45RA and/or granzyme B. In certain aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in certain aspects are carried out simultaneously and in other aspects are carried out sequentially, in either order. In some embodiments, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. An exemplary method is cell sorting and/or selection via negative magnetic immuno-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Expansion of Immune Cells

Whether prior to or after modification of cells to express a subject CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005, the relevant contents of which are incorporated by reference. For example, the immune cells of the invention may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding the immune cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700 fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the immune cells expand in the range of about 20-fold to about 50-fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In particularly exemplary embodiments, the level of confluence is 90% or greater. A period of time can be any time suitable for the culture of cells in vitro. The immune cell medium may be replaced during the culture of the immune cells at any time. In certain exemplary embodiments, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as flt3-L, IL-1, IL-3 and c-kit ligand. In one embodiment, expanding the immune cells comprises culturing the immune cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetylcysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the immune cells expand in the range of about 2-fold to about 50-fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, the contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

### Methods of Treatment

The fusion polypeptides and CARs described herein may be included in a composition for immunotherapy. The composition may include a pharmaceutical composition and further include a pharmaceutically acceptable carrier. A therapeutically effective amount of the pharmaceutical composition comprising the antibodies or antigen binding fragments thereof, scFvs, and modified T cells may be administered.

In one aspect, the invention includes a method for adoptive cell transfer therapy comprising administering to a subject in need thereof a modified T cell of the present invention. In another aspect, the invention includes a method of treating a disease or condition in a subject comprising administering to a subject in need thereof a population of modified T cells. In certain embodiments, the disease is selected from a proliferative disease, a malignancy, a precancerous condition, or a non-cancer related indication associated with expression of disease-related antigen; or a cancer, an atypical and/or a nonclassical cancer, a myelodysplasia, a myelodysplastic syndrome, or a preleukemia.

Also included is a method of treating cancer in a subject in need thereof, comprising administering to the subject an effective amount of a modified immune cell of the invention of the current invention, or a modified immune cell generated by the methods of the invention of the current disclosure, thereby treating the cancer. In some embodiments, the immune cell comprises an isolated nucleic acid encoding an antibody, scFv, or CAR of the invention of the current disclosure. In some embodiments, the modified immune cell is selected from the group consisting of a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) and a dendritic cell. In some embodiments, the antibody, scFv, or CAR of the invention has a binding specificity for human CD19 (hCD19). In some embodiments, the CAR of the invention has a binding specificity for mesothelin. In some embodiments, the cancer is a hematologic cancer. In some embodiments, the cancer is a solid cancer. In some embodiments, the cancer is associated with the expression of CD19. In some embodiments, the CD19 is expressed on tumor cells. In some embodiments, the cancer is selected from the group consisting of Burkitt lymphoma, chronic lymphocytic leukemia (CLL), acute lymphoblastic leukemia (ALL), B-cell lymphoma, and B-cell leukemia. In some embodiments, the subject is a human. In some embodiments, the cancer is associated with expression of mesothelin. In some embodiments, the mesothelin is expressed on tumor cells. In some embodiments, the cancer is mesothelioma, ovarian cancer, lung cancer, squamous carcinoma, non-pulmonary adenocarcinoma, pancreatic cancer, stomach cancer, and colon cancer.

In some embodiments, the cancer is associated with the expression of a tumor antigen. Non-limiting examples of tumor antigens that can be used with the invention include, but are not limited to TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp 100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In one aspect, the present invention provides a method for treating a disease or condition in a subject in need thereof comprising administering to the subject an engineered immune cell or precursor thereof comprising a fusion polypeptide or CAR described herein. The composition can include a pharmaceutical composition and further include a pharmaceutically acceptable carrier. A therapeutically effective amount of the pharmaceutical composition can be administered to the subject.

In certain embodiments, described herein are the modified immune cells or precursors thereof comprising a fusion polypeptide or CAR described herein, for use as a medicament.

In certain embodiments, described herein is use of the modified immune cells or precursors thereof comprising a polypeptide or a CAR described herein for the manufacture of a medicament.

Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

In some embodiments, the method comprises administering to the subject an engineered immune effector cell comprising an isolated fusion polypeptide or CAR comprising an amino acid sequence at least 80%, 85%, 90%, 95%, 96%, 97%, 98%, 99%, or 100% identical to SEQ ID NOs: 1 or 2. In certain embodiments, the immune effector cell is a T cell.

In certain embodiments, the cancer is associated with expression of CD19 or mesothelin. In certain embodiments, the CD19 or mesothelin is expressed on a malignant cell of the subject. In certain embodiments, the malignant cell is associated with B cell acute lymphoblastic leukemia, chronic lymphocytic leukemia, myeloma, mesothelioma, ovarian cancer, lung cancer, squamous carcinoma, non-pulmonary adenocarcinoma, pancreatic cancer, stomach cancer, and colon cancer.

In certain embodiments, the method further comprises administering one or more additional therapeutics or interventions. There is no limitation on such additional therapeutics or interventions, which can include any therapeutic agents or small molecule drugs that is helpful for treating the subject in need thereof. In some embodiments, the additional therapeutics or interventions are administered with the antibody or antigen-binding fragments thereof or the scFv or engineered immune effector cell described herein, or the bispecific molecule or the immunoconjugates comprising these, as a combination therapy. Non-limiting examples of additional therapeutics or interventions include chemotherapy (e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine), radiation therapy, immunotherapy, and other targeted therapy.

Compositions of the present invention can be administered in dosages and routes and at times to be determined in appropriate pre-clinical and clinical experimentation and trials. Compositions can be administered multiple times at dosages within these ranges. Administration of the compositions can be combined with other methods useful to treat the desired disease or condition as determined by those of skill in the art. In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, therapeutically acceptable amount is between about 0.1 mg/kg and about 50 mg/kg. In certain embodiments, therapeutically acceptable amount is between about 1 mg/kg and about 40 mg/kg. In certain embodiments, therapeutically acceptable amount is between about 1 mg/kg and about 20 mg/kg. In certain embodiments, therapeutically acceptable amount is between about 1 mg/kg and about 10 mg/kg. In certain embodiments, therapeutically acceptable amount is between about 5 mg/kg and about 30 mg/kg. In certain embodiments, therapeutically acceptable amount is between about 5 mg/kg and about 20 mg/kg. Therapeutically effective amounts include amounts sufficient to ameliorate one or more symptoms associated with the disease or affliction to be treated.

In terms of the present invention, prophylactic, palliative, symptomatic and/or curative treatments may represent separate aspects of the invention. A fusion polypeptide, CAR, or modified immune cell or precursor thereof comprising a fusion polypeptide or CAR disclosed herein can be administered parenterally, such as intravenously, such as intramuscularly, such as subcutaneously. Alternatively, an antibody or antigen-binding fragments thereof or scFv or CAR or modified immune cell or precursor thereof comprising a fusion polypeptide or CAR of the present invention can be administered via a non-parenteral route, such as orally or topically. An antibody of the invention can be administered prophylactically. An antibody or antigen-binding fragments thereof or scFv or CAR or modified immune cell or precursor thereof comprising a CAR of the present invention can be administered therapeutically (on demand).

### Pharmaceutical Compositions, Kits, and Methods of Making the Compositions

Also provided are pharmaceutical composition comprising any one of the binding polypeptides, scFvs, antibodies, or the antigen-binding fragments disclosed herein. Among the compositions are pharmaceutical compositions and formulations for administration, such as for treatment of a disease or disorder. Also provided are therapeutic methods for administering the pharmaceutical compositions to subjects, e.g., humans.

The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular composition and/or by the method of administration. Accordingly, there are a variety of suitable formulations. For example, the pharmaceutical composition can contain preservatives. Suitable preservatives can include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in an amount effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

In certain embodiments the anti-CD19 antibodies or antigen-binding fragments thereof or scFv or CAR or modified immune cell or precursor thereof comprising an anti-CD19 CAR of the current invention are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current invention are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current invention can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration.

In certain embodiments, described herein is a method of making a composition for treating cancer, sepsis or septic shock, or chronic infection (e.g., viral infections), comprising admixing one or more pharmaceutically acceptable excipients, carriers, or diluents and an antibody of the current invention. In certain embodiments, described herein is a method of preparing a cancer treatment for storage or shipping comprising lyophilizing one or more antibodies of the current invention.

While the present invention has been described with reference to the specific embodiments thereof, it should be understood by those skilled in the art that various changes can be made, and equivalents can be substituted without departing from the true spirit and scope of the present invention. It will be readily apparent to those skilled in the art that other suitable modifications and adaptations of the methods described herein can be made using suitable equivalents without departing from the scope of the embodiments disclosed herein. In addition, many modifications can be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The present invention is further described in detail by reference to the following experimental examples. These examples are provided for purposes of illustration only and are not intended to be limiting unless otherwise specified. Thus, the present invention should in no way be construed as being limited to the following examples, but rather, should be construed to encompass any and all variations which become evident as a result of the teaching provided herein. While preferred embodiments of the present invention have been shown and described herein, it is understood by those skilled in the art that such embodiments are provided by way of example only. Numerous variations, changes, and substitutions will now occur to those skilled in the art without departing from the present invention. It should be understood that various alternatives to the embodiments of the present invention described herein can be employed in practicing the present invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this invention.

Without further description, it is believed that one of ordinary skill in the art can, using the preceding description and the following illustrative examples, make and utilize the compounds of the present invention and practice the claimed methods. The following working examples, therefore, specifically point out some embodiments of the present invention.

### Example 1: Down-Regulation of CAR Expression

Durable CAR expression correlates with durable patient responses. CAR surface expression is regulated, in part, by ligand-induced downregulation. The studies disclosed herein sought to increase and prolong the surface expression of CARs by co-expressing the small GTPases Rab5 or Rab11, which are important for directing endocytosed material back to the cell surface and away from the lysosome. Here, the studies expressed Rab5 or Rab11 in CAR-T cells and showed that an increased percentage of T-cells with surface CAR expression and showed increased killing after repeated exposure to antigen.

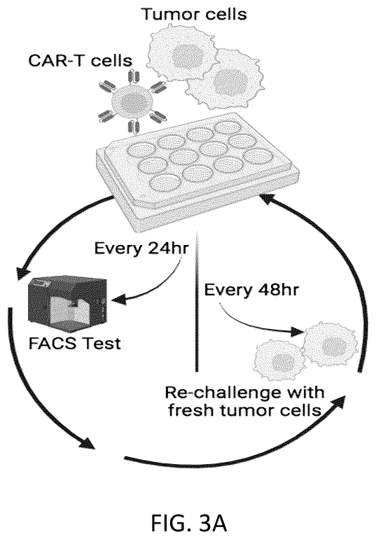

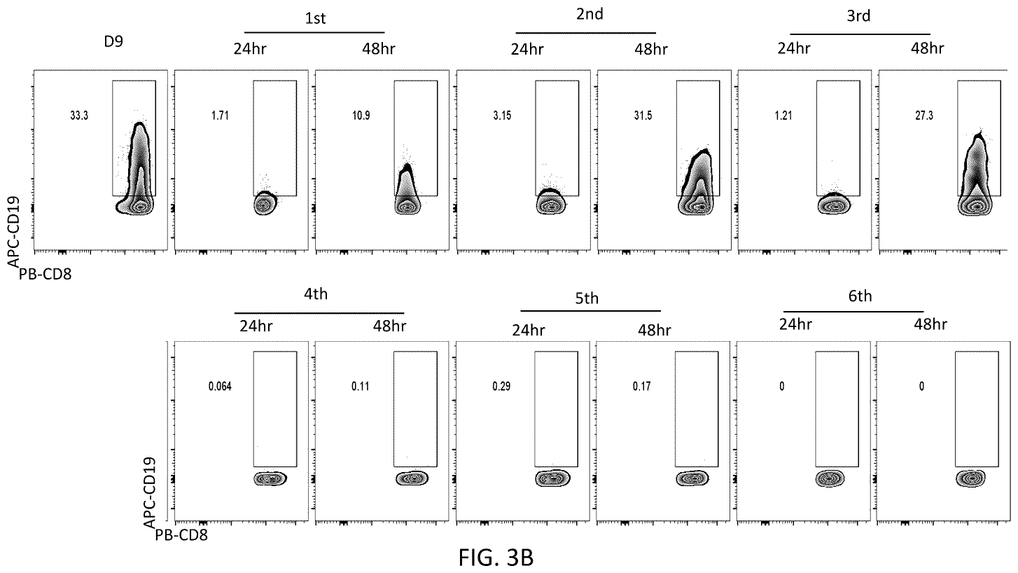

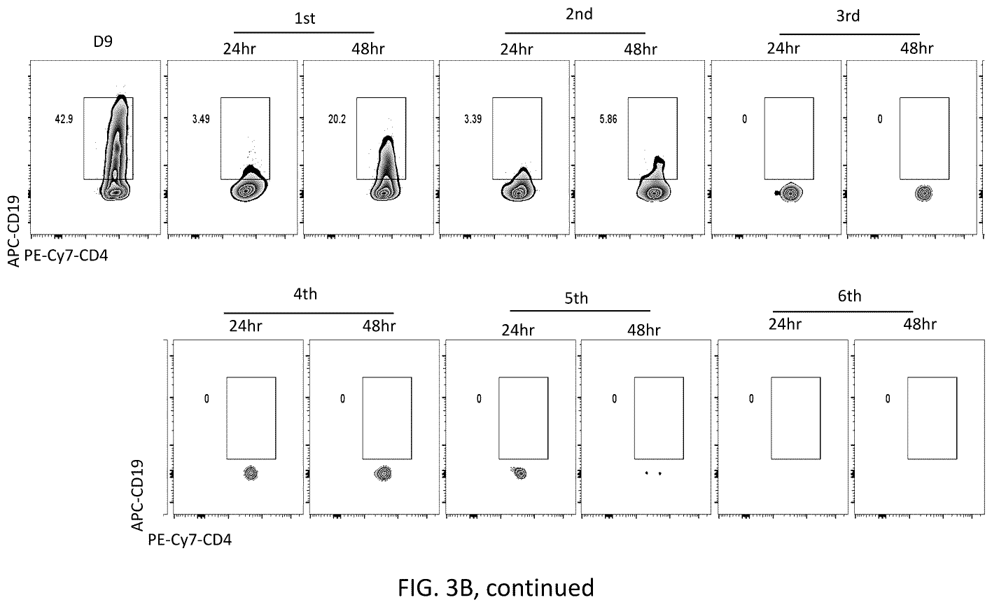

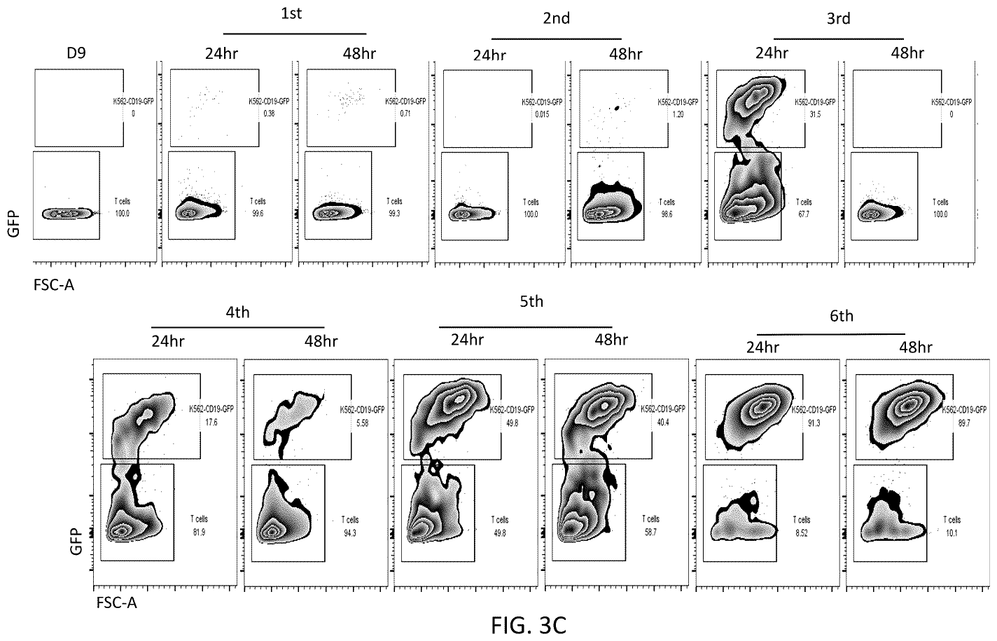

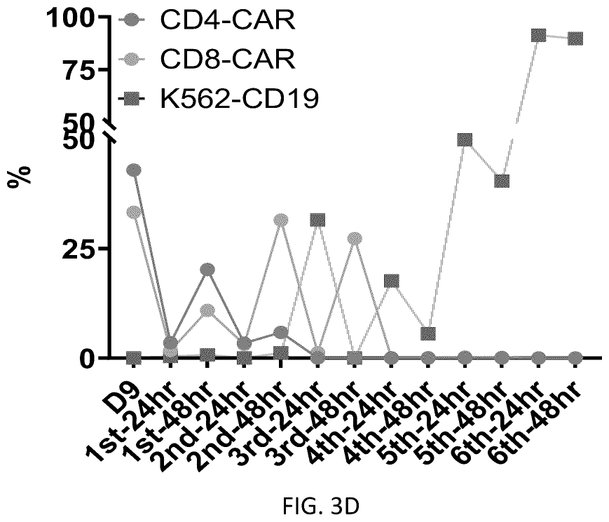

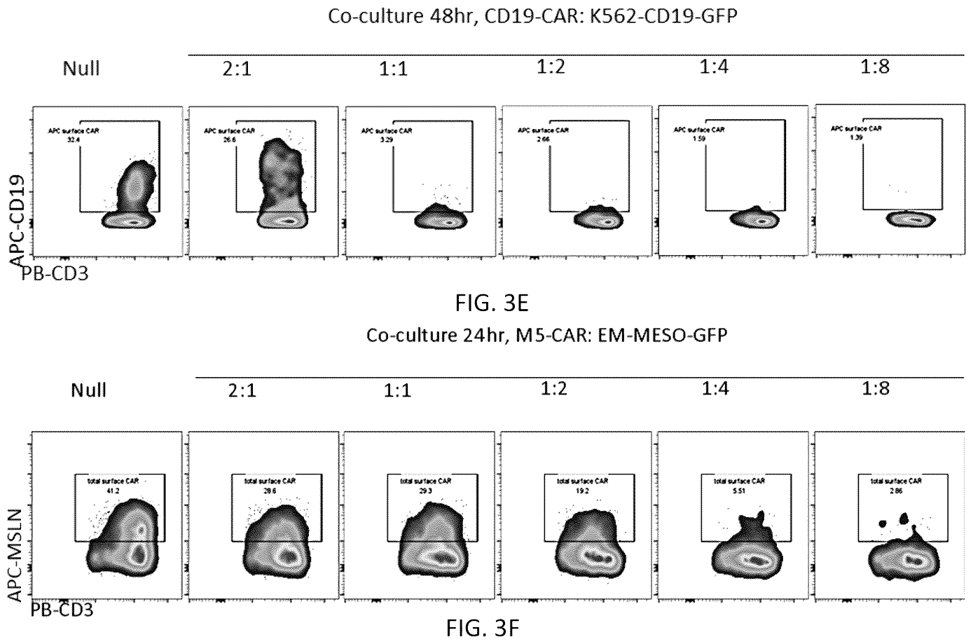

In an initial study, it was sought to observe the downregulation of CAR expression after repeated tumor challenge or at low effector to target ratios. Primary human T cells were activated with CD3/28 coated beads transduced with a lentiviral vector that expresses a CD19 CAR linked to 4-1BB and CD3 zeta signaling domains. After 9 days of expansion these cells were cultured with K562 cells expressing CD19 as indicated in FIG. 3A. Surface CAR expression level was determined by flow cytometry using APC-labelled CD19 on the indicated day as indicated in FIG. 3B. GFP+CD19+K562 cells population was analyzed in the co-cultured described in FIG. 3C. FIG. 3D shows an overlay of surface CAR expression profile and tumor cell survive profile summarized from (FIG. 3B) and (FIG. 3C). Finally, in FIGS. 3E-3F, human CD19 or mesothelin CAR T cells were generated as described and cocultured with GFP+CD19+K562 cells or EM-MESO-GFP+ cells as indicated effector to target ratio. Together these results demonstrated that CAR expression decreases with continued antigen exposure.

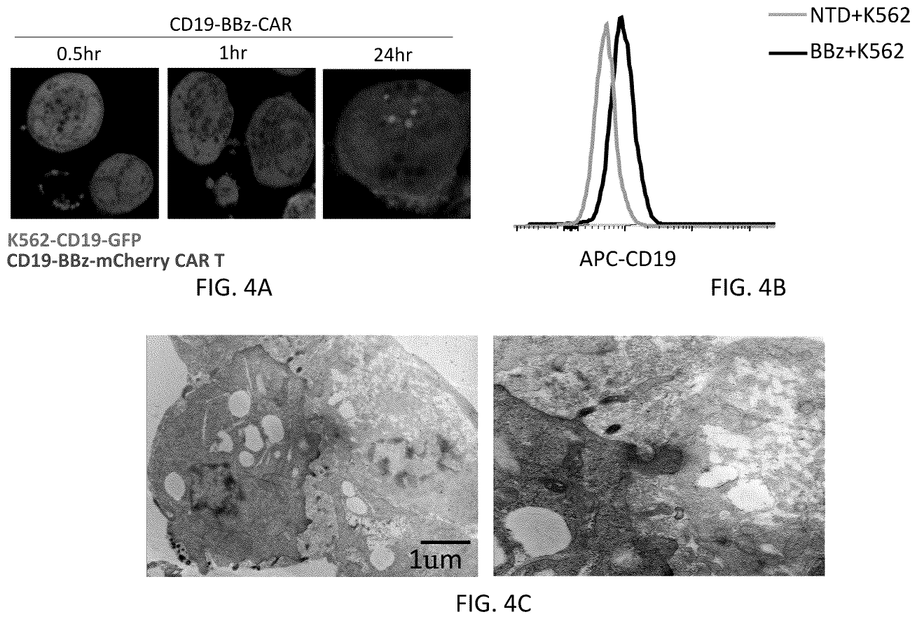

Loss of CAR expression could be due to a number of mechanisms. Without wishing to be bound by theory, one mechanism is that Rab5 enhances the recycling of endocytosed CARs back to the membrane in CAR T cells. Another possible reason for CAR construct loss is the uptake of CAR proteins by tumor cells, which would essentially “rip” the complexes from responding T cell after antigen binding. Both mechanisms could contribute to CAR the observed loss of CAR complex expression following antigen recognition or repeated antigen recognition. To assess these potential phenomena, a series of studies was undertaken in which primary human cells were transduced with a lentiviral vector encoding CD19−BBz CAR fused with the mCherry. After 9 days of culture, these cells were mixed CD19+K562 cells expressing GFP, and these cells were cocultured as the indicated time point and CAR location was visualized using confocal microscopy. See FIG. 4B. Co-cultures were permeabilized and stained with APC-labeled CD19 protein 24 hr after the 3rd cycle of coculture with null transduced human T cells (gray), CD19−BBz CAR-expression human T cells (black) and CAR expression visualized by flow cytometry. FIG. 4C shows in representative TEM images that CARs are released from T cells cross synapse. Human T cell expressing CD19−BBz-APEX2 CAR (dark) incubate with GFP+CD19+K562 cells (light) for overnight coculture. Scale bar is 1 μm (left) and 500 nm (right). In FIG. 4D, representative TEM images show that CAR complexes are up taken into tumor cells. Human T cell null transducing or expressing CD19−BBz-APEX2 incubate with GFP+CD19+K562 cells. 24 hr after the 3rd cycle of coculture, T cells were removed by CD3+ beads, TEM analysis GFP+CD19+K562 cells containing CD19−BBz-APEX2 CAR (black) in the vesicles. Scale bar is 1 um. Together, these studies indicated that CAR complex downregulation can occur due to a number of mechanisms, including downregulation of expression by the T cell, as well as transfer of the CAR complex to the tumor cell after contact.

### Example 2: Maintenance of CAR Expression by Co-Expression of Rab Proteins

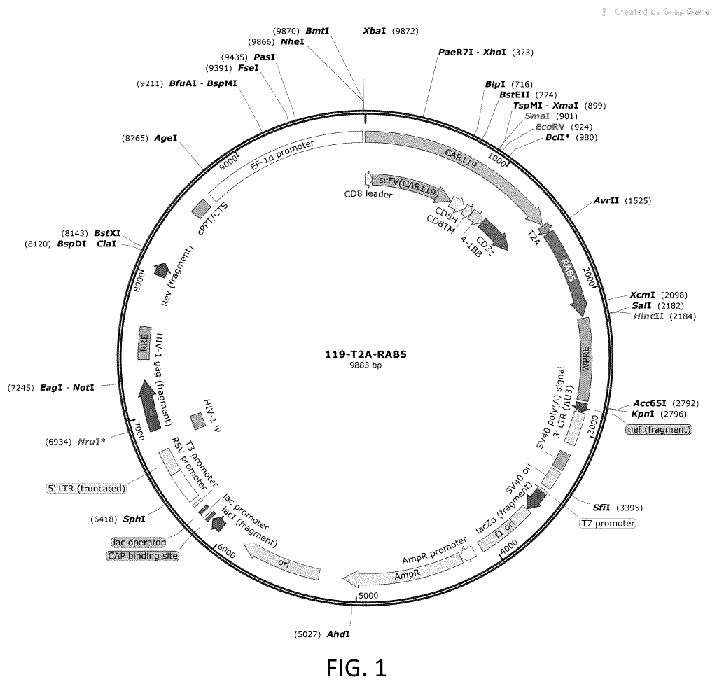

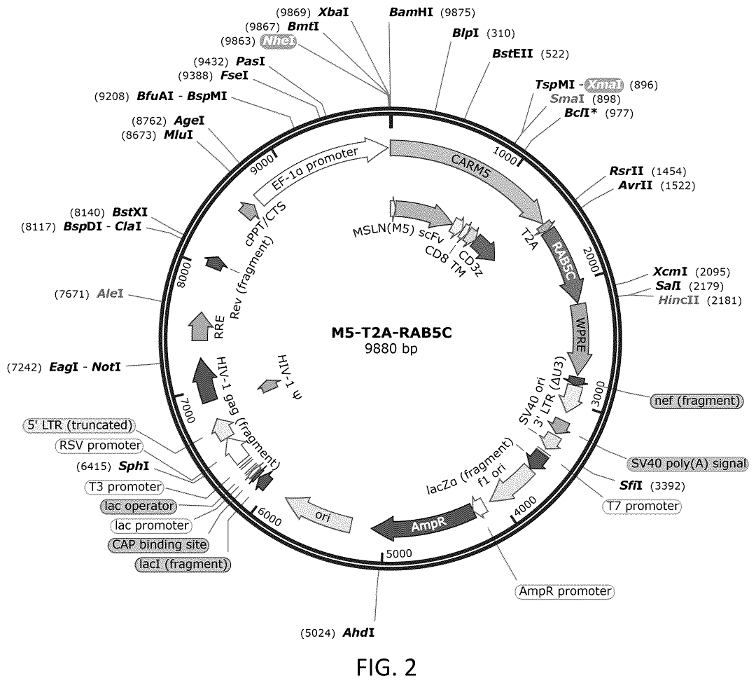

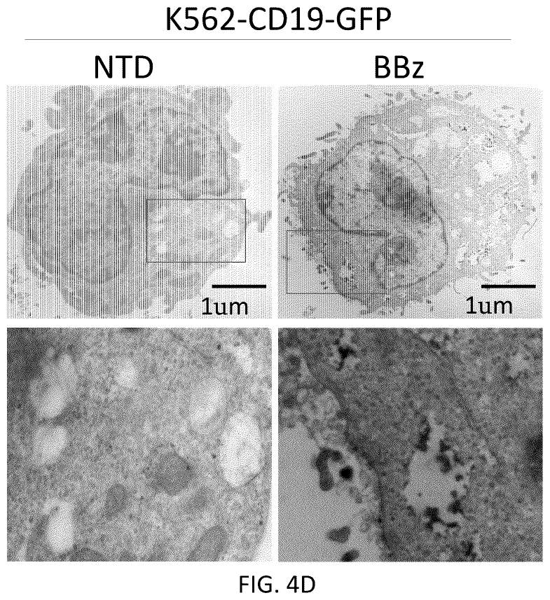

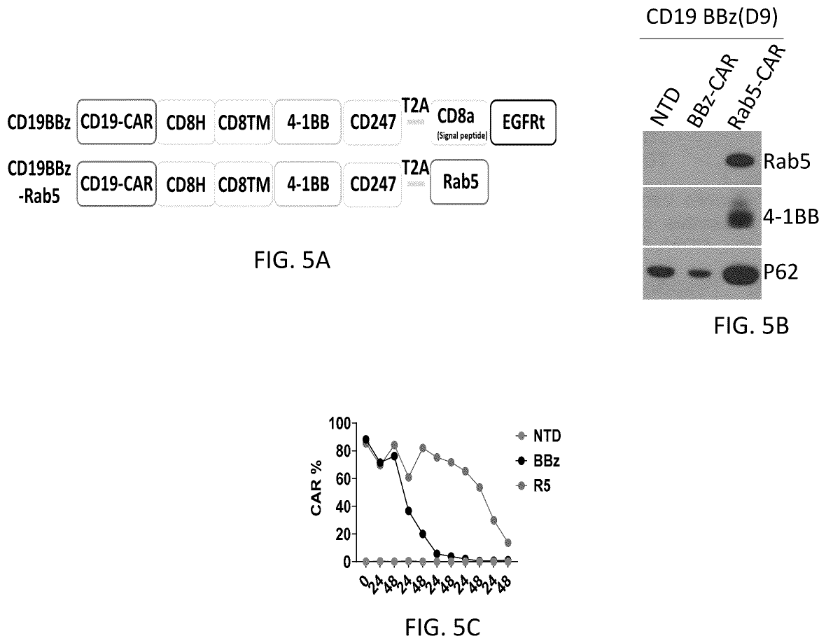

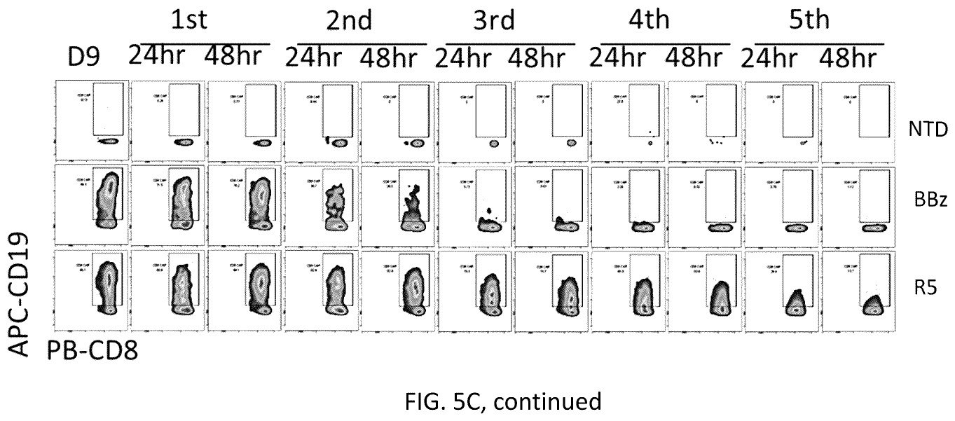

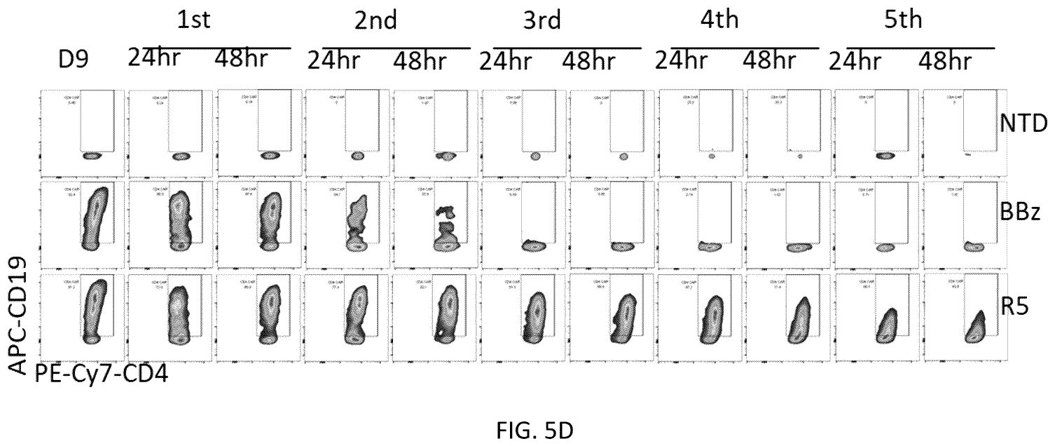

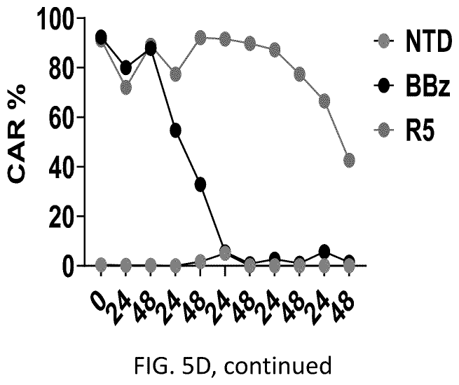

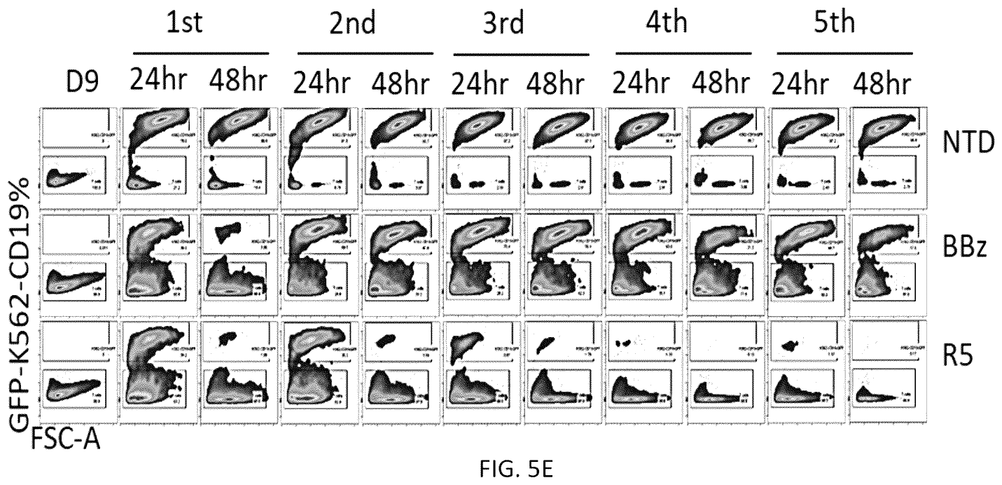

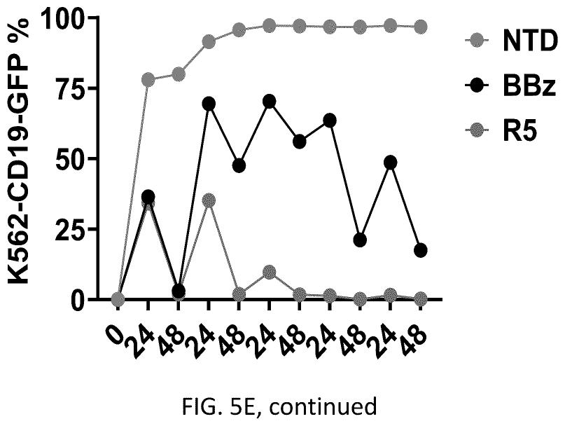

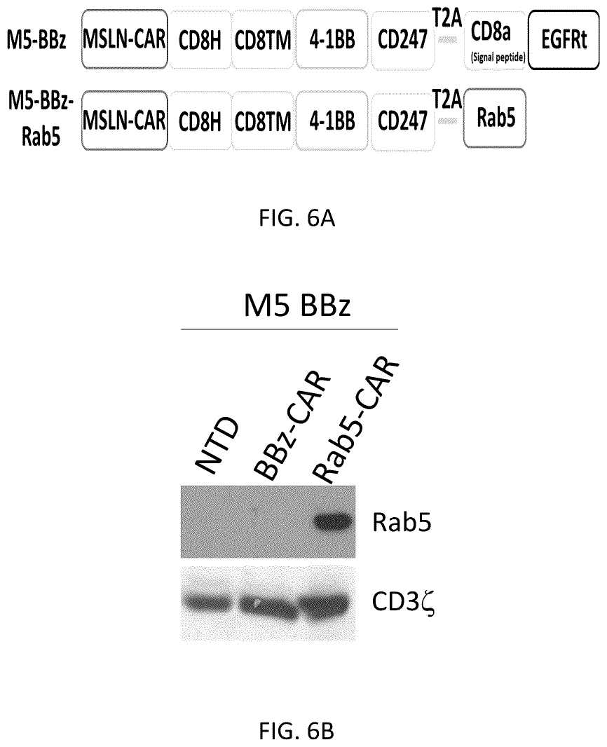

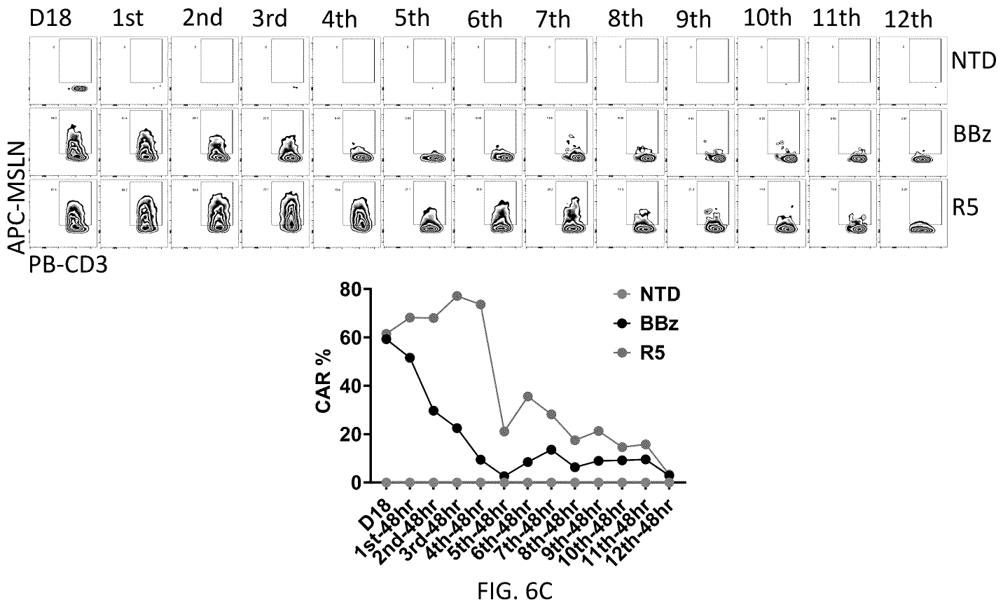

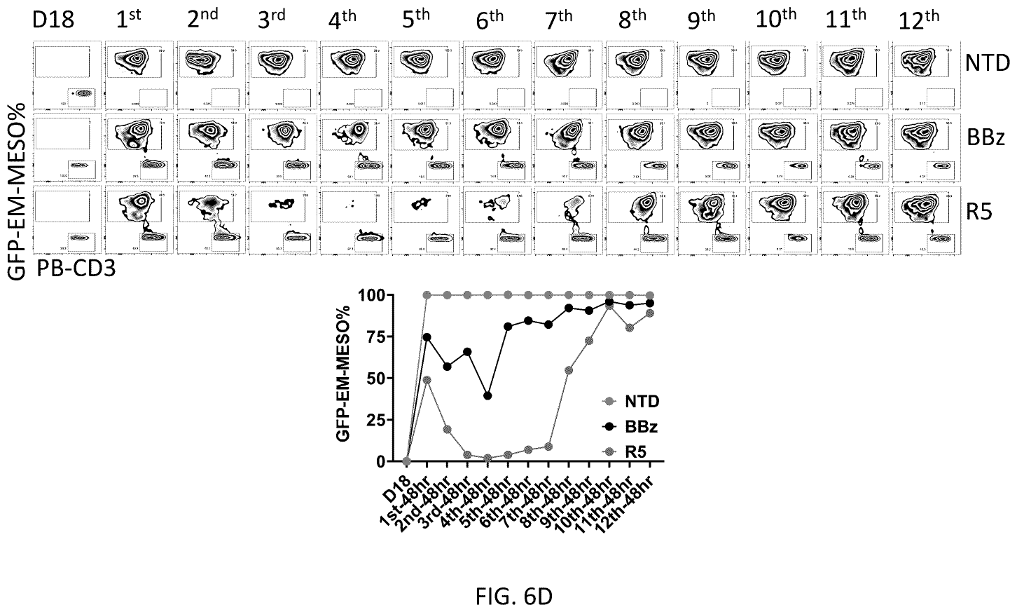

In order to maintain expression of CAR constructs in CAR T cells, a series of studies was conducted to see whether the co-expression of the CAR receptor with Rab proteins could improve or maintain surface CAR expression. A series of CAR constructs were created in which the CAR and Rab5 protein were expressed as a single polypeptide separated by a cleavable T2A linker. Upon expression in the T cell, the T2A linker is cleaved separating the CAR and the RAB5 protein. Maps of 2 such fusion constructs, one specific for CD19 and another specific for mesothelin are illustrated in FIG. 1 and FIG. 2. Schematics of these CARs are also shown in FIG. 5A and FIG. 6A. In FIG. 5B, primary human T cells were transduced with the indicated vector expressing CD19 CAR and cultured for 9 days. Immunoblot analysis of RAB5 protein levels is displayed. The experiment described in Example 1 was then repeated with the addition of CAR T cells that co-expressed RAB5, the results of which demonstrated that RAB5 co-expression improved CAR expression (FIG. 5C-5D). Similar studies were conducted with the mesothelin CAR, which are illustrated in FIGS. 6A-6D. Together these studies demonstrated that engineering MSLN-CAR with RAB5 increased its persistence and activity under repeated tumor stimulation.

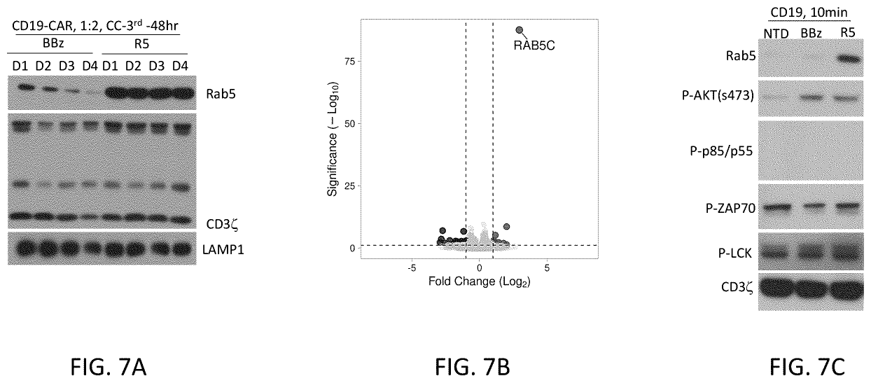

A series of studies then sought to determine whether RAB5 promotes surface CAR persistence directly. FIG. 7A show an immunoblot analysis of CAR level in CD19−BBz CAR or CD19−BBz-RAB5 CAR-expression human T cells isolated from four different donors (D1-D4). CD19−CAR T cells incubate with GFP+CD19+K562 cells. 48 hr after the 3rd cycle of coculture, T cells were isolated by FACS sorting for immunoblotting. FIG. 7B. Volcano plot of RNA sequencing analysis of differentially expressed genes in CD19−BBz CAR-expression human T cells relative to CD19−BBz-RAB5 CAR-expression human T cells. In FIG. 7C, an immunoblot analysis of CD19 demonstrates induced signaling pathway in CD19−BBz CAR-T and CD19−BBz-RAB5 CAR-T cells. Together these studies demonstrated that RAB5 promotes surface CAR persistence directly.

### Example 3: Altering GTPase Activity of Rab5 to Effect CAR Expression

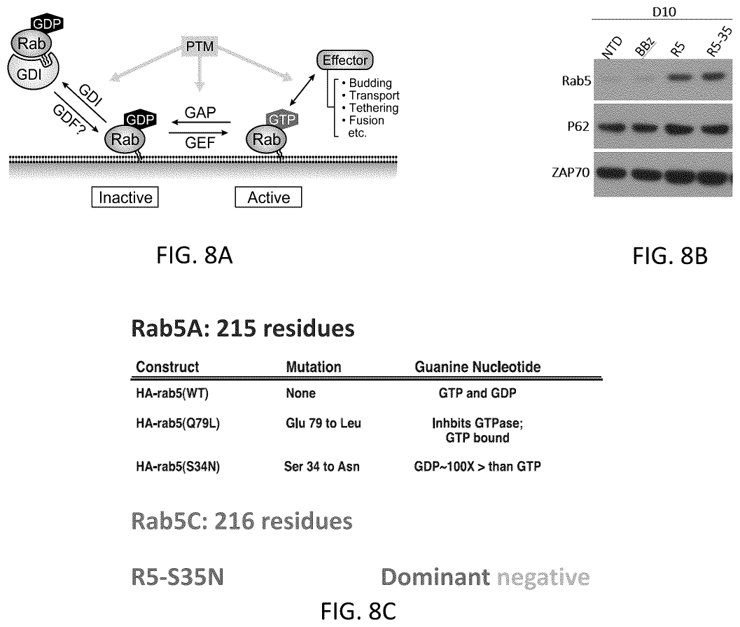

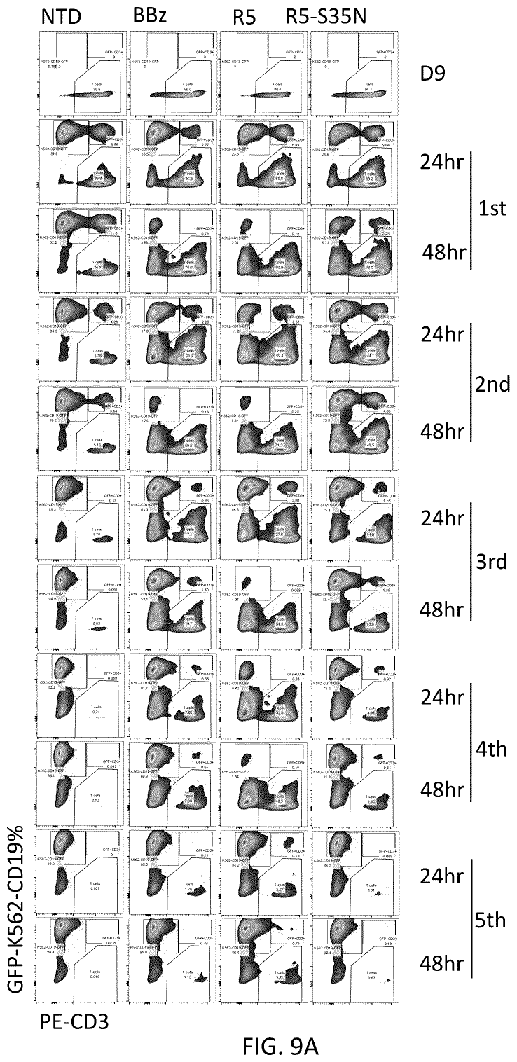

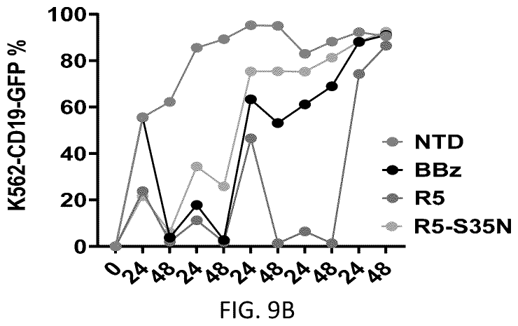

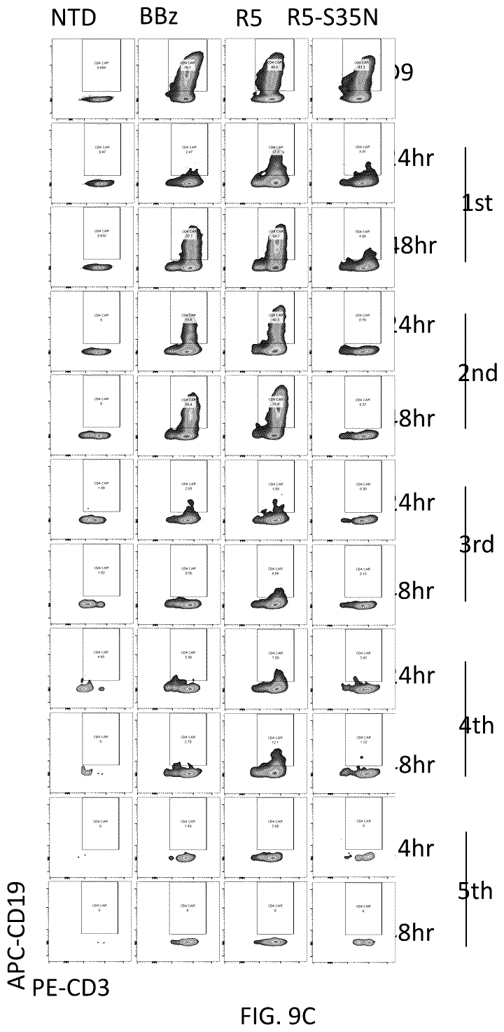

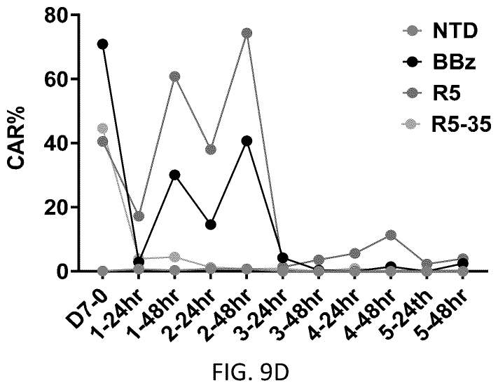

Next, a series of studies sought to determine altering the GTPase activity of Rab5 would also alter its ability to modulate CAR expression. FIGS. 8A and 8C illustrate the Rab GTPase cycle mechanism and RAB5 dominant negative vector design. FIG. 8B is an immunoblot analysis of protein level of wild-type or mutant RAB5 in CD19−CAR-T cells. The importance of the GTPase function of Rab5 is illustrated in FIG. 9, which shows that RAB5 enables CAR expression dependent on its GTPase function. Here a FACS analysis and quantification of GFP+CD19+K562 cells population and surface CAR expression level was conducted. CD19−BBz CAR, CD19−BBz-RAB5 CAR and CD19−BBz-RAB5-S35N CAR-expression human T cells were coculture with GFP+CD19+K562 cells as shown previously.

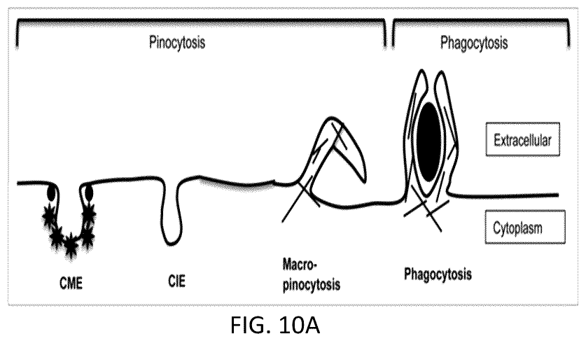

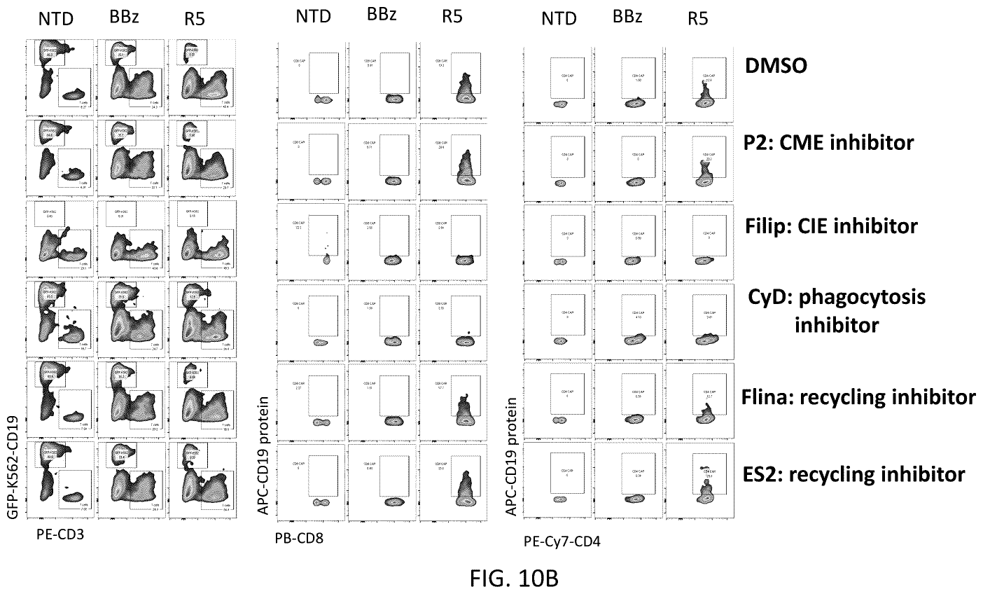

Next, a series of studies demonstrated that Rab5 promotes surface CAR endocytosis. FIG. 10A illustrates the different types of endocytosis. CME: clathrin-mediated endocytosis. CIE: clathrin-independent endocytosis. In FIG. 10B, FACS analysis of surface CAR expression in the presence of indicated inhibitors is illustrated. Inhibitors used in this study included P2, an inhibitor of clathrin-mediated endocytosis; filip, an inhibitor of clathrin independent endocytosis; cytochalasin D (CyD) an inhibitor of actin polymerization and therefore phagocytosis; and flina and Endosidin2 (ES2) which are inhibitors of vesicle recycling to the cell surface. CD19−BBz CAR or CD19−BBz-RAB5 CAR-expression human T cells were coculture with GFP+CD19+K562 cells, 24 hr after the 3rd cycle of coculture, CAR-T cells were treated with inhibitors for additional 24 hr, the surface CAR level was detected by APC-CD19 staining.

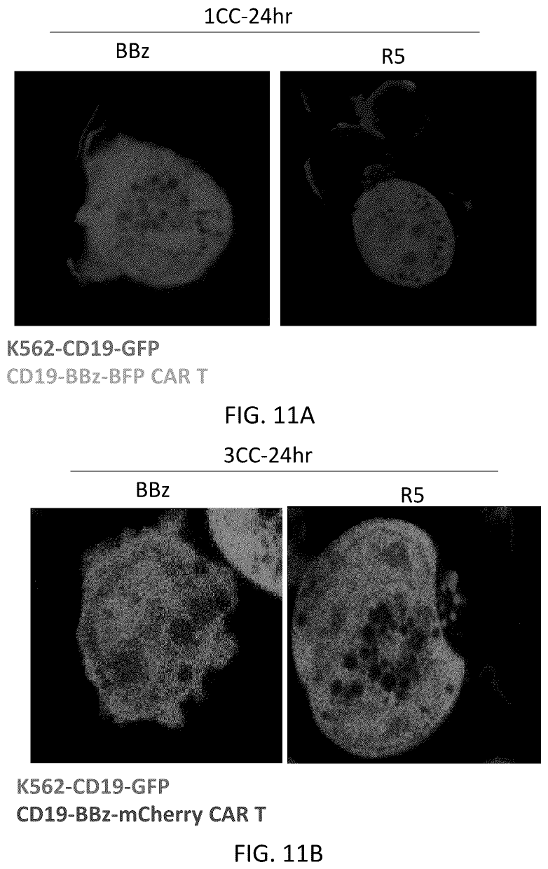

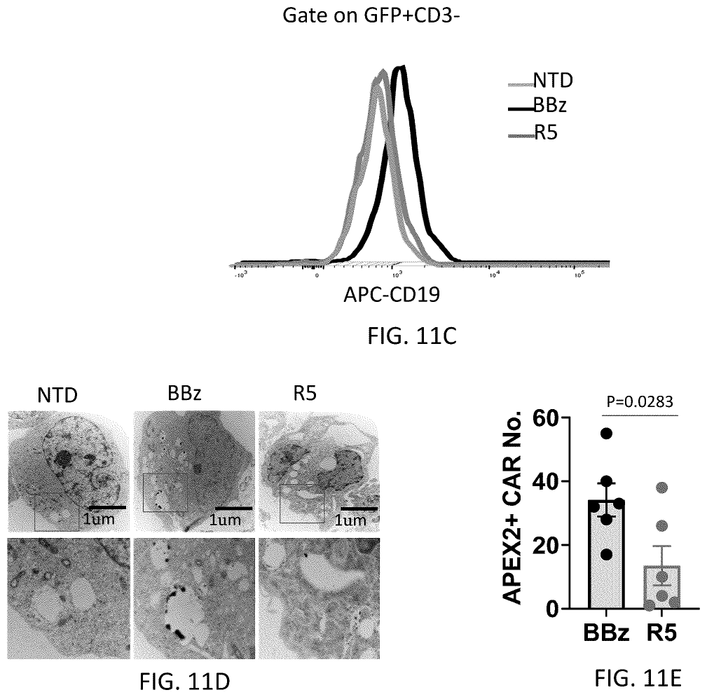

Studies then turned to exploring the mechanism by which Rab5 expression maintains CAR construct expression on the surface of modified T cells. FIGS. 11A-B are representative confocal images showing CAR location after recognizing tumor cells. CD19−BBz (BBz) and CD19−BBz-RAB5 (R5) CAR fused BFP (blue) (FIG. 11A) or mCherry (red) (FIG. 11B), CD19+K562 cells fused GFP (green), coculture as the indicated time point. FIG. 11C is a FACS analysis of CAR expression level inside of GFP+CD19+K562 cells. Intracellular staining of CAR with tumor cells 24 hr after the 3rd cycle of coculture with null transduced human T cells (gray), CD19−BBz CAR-expression human T cells (black) or CD19−BBz-RAB5 CAR-expression human T cells (green) separately. Finally, FIG. 11D shows representative TEM images showing CAR is up taken into the vehicles of tumor cells. Human T cell null transducing, expressing CD19−BBz-APEX2 or CD19−BBz-APEX2-RAB5 incubate with GFP+CD19+K562 cells. 24 hr after the 3rd cycle of coculture, T cells were deleted by CD3+ beads, TEM analysis GFP+CD19+K562 cells containing APEX2 CAR (black) in the vesicles. Scale bar is 1 um. These results are quantified in FIG. 11E, where each dot represents a single GFP+CD19+K562 cell. These results demonstrated that Rab5 co-expression with the CAR construct can reduce uptake of the CAR complex into the tumor cell and maintain surface expression on the T cell. Without wishing to be bound by theory, these studies suggested that Rab5 enhances the recycling of endocytosed CARs back to the membrane in CAR T cells, thereby improving CAR membrane expression persistence and CAR T cell cytotoxicity. This appears to be the prevalent mechanism because a dominant negative Rab5 (S35N) suppressed the enhancement (FIGS. 8-9). The role of Rab small GTPases in modulating membrane receptor trafficking is known, but it has never been shown to enhance CAR T cell activity.

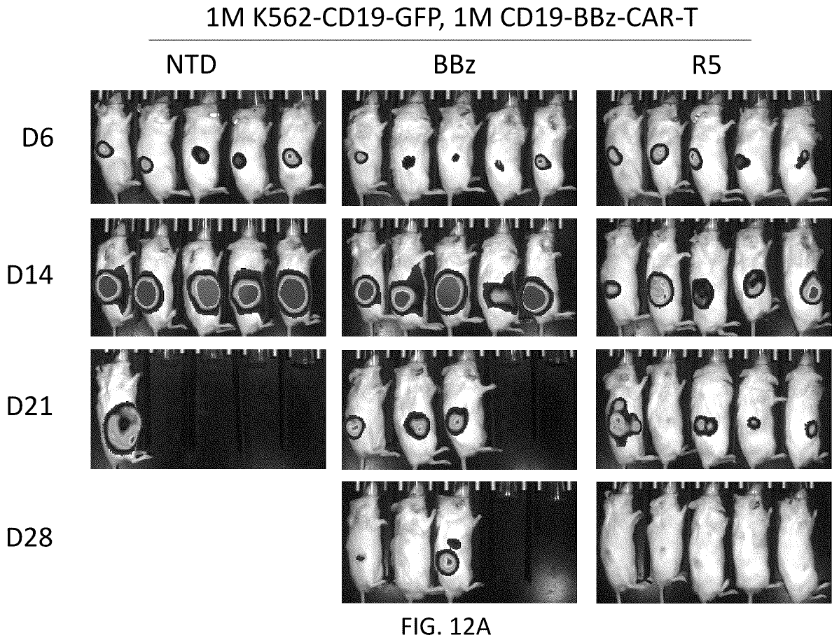

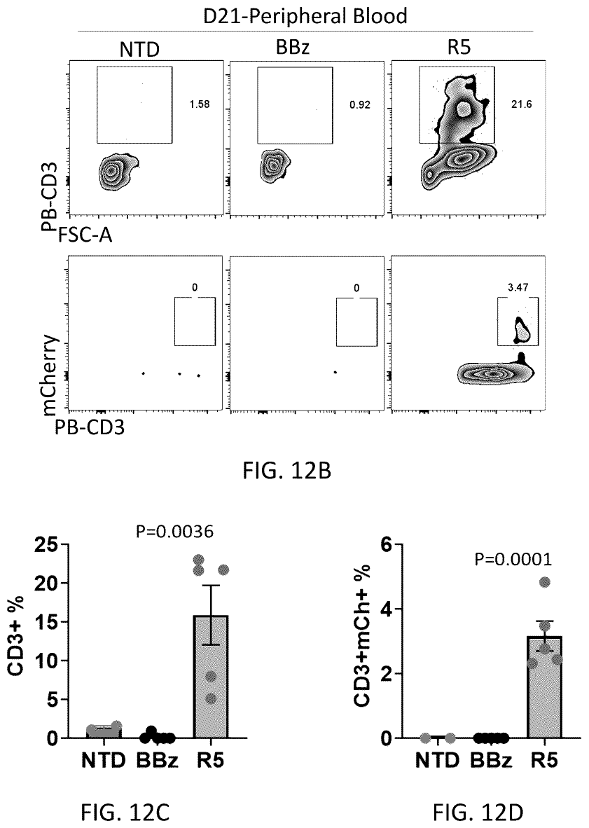

Studies were then conducted to determine if the co-expression of Rab5, and the associated enhancement of CAR surface expression, could result in improved CAR cytotoxic function. Here, a million GFP T2A luciferase CD19+K562 cells were implanted in NSG mice. 7 days later, 1M of the indicated CAR-T cells linked to mCherry were adoptive transferred to the tumor bearing mice by i.v. Bioluminescence imaging of the mice was performed on the indicated days (see FIG. 12A). In FIGS. 12B-12D, Peripheral blood was obtained 21 days after CAR T cell infusion from surviving NSG mice and the number of total T cells (top) and CAR T cells (bottom) by flow cytometry. Data is summarized in FIG. 12C with each dot representing the data obtained from a mouse. Together, these data illustrated that Rab5 co-expression can be used to maintain CAR surface expression on CAR T cells, which in turn can maintain the cytotoxic function of the cells, leading to improved antitumor efficacy.

### ENUMERATED EMBODIMENTS

The following enumerated embodiments are provided, the numbering of which is not to be construed as designating levels of importance.

Embodiment 1 provides a fusion protein comprising a chimeric antigen receptor linked to a second polypeptide by a linker domain, wherein the CAR comprises an antigen binding domain, a transmembrane domain, and an intracellular domain, and wherein the second polypeptide prolongs the surface expression of the CAR, and wherein the linker is a cleavable linker.

Embodiment 2 provides the fusion protein of embodiment 1, wherein the second polypeptide is a Rab5 protein.

Embodiment 3 provides the fusion protein of embodiment 1, wherein the second polypeptide is a Rab11 protein.

Embodiment 4 provides the fusion protein of embodiment 1, wherein the linker is a self-cleaving peptide.

Embodiment 5 provides the fusion protein of embodiment 4, wherein the self-cleaving peptide is a 2A peptide.

Embodiment 6 provides the fusion protein of embodiment 1, wherein the CAR has a binding specificity for a cancer-related antigen.

Embodiment 7 provides the fusion protein of embodiment 6, wherein the cancer-related antigen is CD19.

Embodiment 8 provides the fusion protein of embodiment 6, wherein the cancer-related antigen is mesothelin.

Embodiment 9 provides the fusion protein of embodiment 6, wherein the cancer-related antigen is selected from a group consisting of: TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

Embodiment 10 provides the fusion protein of embodiment 1, wherein the intracellular domain comprises a signaling domain selected from the group consisting of a 4-1BB signaling domain, a CD28 signaling domain, and a KIR signaling domain.

Embodiment 11 provides the fusion protein of embodiment 10, wherein the 4-1BB signaling domain comprises the amino acid sequence set forth in SEQ ID NO: 8.

Embodiment 12 provides the fusion protein of embodiment 1, wherein the intracellular domain comprises a CD3zeta signaling domain.

Embodiment 13 provides the fusion protein of embodiment 12, wherein the CD3zeta domain comprises an amino acid sequence set forth in SEQ ID NO: 9.

Embodiment 14 provides the fusion protein of embodiment 10, wherein the signaling domain is a KIR2DS2 domain and comprises the amino acid sequence set forth in SEQ ID NO: 41.

Embodiment 15 provides the fusion protein of embodiment 1, further comprising a CD8 leader sequence.

Embodiment 16 provides the fusion protein of embodiment 15, wherein the CD8 leader sequence comprises the amino acid sequence set forth in SEQ ID NO: 4.

Embodiment 17 provides the fusion protein of embodiment 1, wherein the transmembrane domain is a CD8 transmembrane domain.

Embodiment 18 provides the fusion protein of embodiment 17, wherein the CD8 transmembrane domain comprises the amino acid sequence set forth in SEQ ID NO: 5.

Embodiment 19 provides the fusion protein of embodiment 1, wherein the fusion protein comprises the amino acid sequence set forth in SEQ ID NO: 1.

Embodiment 20 provides the fusion protein of embodiment 1, wherein the fusion protein comprises the amino acid sequence set forth in SEQ ID NO: 2.

Embodiment 21 provides the fusion protein of embodiment 1, wherein the second polypeptide comprises the amino acid sequence set forth in SEQ ID NO: 3.

Embodiment 22 provides the fusion protein of embodiment 1, wherein the cleavable linker comprises the amino acid sequence set forth in SEQ ID NO: 10.

Embodiment 23 provides the fusion protein of embodiment 1, wherein the CAR comprises the amino acid sequence set forth in SEQ ID NOs: 1-2 or 43-85.

Embodiment 24 provides a nucleic acid vector encoding the modified fusion protein of embodiment 1.

Embodiment 25 provides the vector of embodiment 24, wherein the vector is an expression vector.

Embodiment 26 provides a modified immune cell or precursor thereof, comprising the fusion protein of embodiment 1 or the nucleic acid vector of embodiment 24.

Embodiment 27 provides the modified immune cell of embodiment 26, wherein the immune cell is selected from the group consisting of a T cell, a macrophage, and a NK cell.

Embodiment 28 provides the modified immune cell of embodiment 26, wherein the immune cell is a T cell.

Embodiment 29 provides the modified immune cell of embodiment 28, wherein the T cell is selected from the group consisting of a CD4 T cell, a CD8 T cell, and a NK T cell.

Embodiment 30 provides the modified immune cell of embodiment 29, wherein the T cell is a CD8 T cell.

Embodiment 31 provides a method of treating cancer in a subject in need thereof, comprising administering to the subject an effective amount of a modified immune cell comprising a fusion protein comprising a chimeric antigen receptor linked to a second polypeptide by a linker domain;

wherein the CAR comprises an antigen binding domain, a transmembrane domain, and an intracellular domain, and wherein the second polypeptide prolongs the surface expression of the CAR, and wherein the linker is a cleavable linker.

Embodiment 32 provides the method of embodiment 31, wherein the second polypeptide is a Rab5 protein.

Embodiment 33 provides the method of embodiment 31, wherein the second polypeptide is a Rab11 protein.

Embodiment 34 provides the method of embodiment 31, wherein the linker is a self-cleaving peptide.

Embodiment 35 provides the fusion protein of embodiment 34, wherein the self-cleaving peptide is a 2A peptide.

Embodiment 36 provides the method of embodiment 31, wherein the CAR has a binding specificity for a cancer-related antigen.

Embodiment 37 provides the method of embodiment 36, wherein the cancer-related antigen is CD19.

Embodiment 38 provides the method of embodiment 36, wherein the cancer-related antigen is mesothelin.

Embodiment 39 provides the method of embodiment 36, wherein the cancer-related antigen is selected from a group consisting of: TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

Embodiment 40 provides the method of embodiment 31, wherein the intracellular domain comprises a signaling domain selected from the group consisting of a 4-1BB signaling domain, a CD28 signaling domain, and a KIR2DS2 domain.

Embodiment 41 provides the method of embodiment 40, wherein the 4-1BB signaling domain comprises the amino acid sequence set forth in SEQ ID NO: 8.

Embodiment 42 provides the method of embodiment 40, wherein the KIR2DS2 domain comprises the amino acid sequence set forth in SEQ ID NO: 41.

Embodiment 43 provides the method of embodiment 31, wherein the intracellular domain comprises a CD3zeta signaling domain.

Embodiment 44 provides the method of embodiment 42, wherein the CD3zeta domain comprises an amino acid sequence set forth in SEQ ID NO: 9.

Embodiment 45 provides the method of embodiment 31, further comprising a CD8 leader sequence.

Embodiment 46 provides the method of embodiment 45, wherein the CD8 leader sequence comprises the amino acid sequence set forth in SEQ ID NO: 4.

Embodiment 47 provides the method of embodiment 31, wherein the transmembrane domain is a CD8 transmembrane domain.

Embodiment 48 provides the method of embodiment 47, wherein the CD8 transmembrane domain comprises the amino acid sequence set forth in SEQ ID NO: 5.

Embodiment 49 provides the method of embodiment 31, wherein the fusion protein comprises the amino acid sequence set forth in SEQ ID NO: 1.

Embodiment 50 provides the method of embodiment 31, wherein the fusion protein comprises the amino acid sequence set forth in SEQ ID NO: 2.

Embodiment 51 provides the method of embodiment 31, wherein the second polypeptide comprises the amino acid sequence set forth in SEQ ID NO: 3.

Embodiment 52 provides the method of embodiment 31, wherein the cleavable linker comprises the amino acid sequence set forth in SEQ ID NO: 10.

Embodiment 53 provides the method of embodiment 31, wherein the CAR comprises an amino acid sequence selected from the group consisting of SEQ ID NOs: 1-2 and 43-85.

Embodiment 54 provides the method of embodiment 31, wherein the cancer is selected from the group consisting of B cell acute lymphoblastic leukemia, chronic lymphocytic leukemia, myeloma, mesothelioma, ovarian cancer, lung cancer, squamous carcinoma, non-pulmonary adenocarcinoma, pancreatic cancer, stomach cancer, and colon cancer.

Embodiment 55 provides the method of embodiment 31, wherein the subject is human.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention can be devised by others skilled in the art without departing from the true spirit and scope of the present invention. The appended claims are intended to be construed to include all such embodiments and equivalent variations.

What is claimed is:

1. A fusion protein comprising a chimeric antigen receptor linked to a second polypeptide by a linker domain, wherein the CAR comprises an antigen binding domain, a transmembrane domain, and an intracellular domain, and wherein the second polypeptide prolongs the surface expression of the CAR, and wherein the linker is a cleavable linker.

2. The fusion protein of claim 1, wherein the second polypeptide is a Rab5 protein.

3. The fusion protein of claim 1, wherein the second polypeptide is a Rab11 protein.

4. The fusion protein of claim 1, wherein the linker is a self-cleaving peptide.

5. The fusion protein of claim 4, wherein the self-cleaving peptide is a 2A peptide.

6. The fusion protein of claim 1, wherein the CAR has a binding specificity for a cancer-related antigen.

7. The fusion protein of claim 6, wherein the cancer-related antigen is CD19.

8. The fusion protein of claim 6, wherein the cancer-related antigen is mesothelin.

9. The fusion protein of claim 6, wherein the cancer-related antigen is selected from a group consisting of: TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

10. The fusion protein of claim 1, wherein the intracellular domain comprises a signaling domain selected from the group consisting of a 4-1BB signaling domain, a CD28 signaling domain, and a KIR signaling domain.

11. The fusion protein of claim 10, wherein the 4-1BB signaling domain comprises the amino acid sequence set forth in SEQ ID NO: 8.

12. The fusion protein of claim 1, wherein the intracellular domain comprises a CD3zeta signaling domain.

13. The fusion protein of claim 12, wherein the CD3zeta domain comprises an amino acid sequence set forth in SEQ ID NO: 9.

14. The fusion protein of claim 10, wherein the KIR signaling domain is a KIR2DS2 domain and comprises the amino acid sequence set forth in SEQ ID NO: 41.

15. The fusion protein of claim 1, further comprising a CD8 leader sequence.

16. The fusion protein of claim 15, wherein the CD8 leader sequence comprises the amino acid sequence set forth in SEQ ID NO: 4.

17. The fusion protein of claim 1, wherein the transmembrane domain is a CD8 transmembrane domain.

18. The fusion protein of claim 17, wherein the CD8 transmembrane domain comprises the amino acid sequence set forth in SEQ ID NO: 5.

19. The fusion protein of claim 1, wherein the fusion protein comprises the amino acid sequence set forth in SEQ ID NO: 1.

20. The fusion protein of claim 1, wherein the fusion protein comprises the amino acid sequence set forth in SEQ ID NO: 2.

21. The fusion protein of claim 1, wherein the second polypeptide comprises the amino acid sequence set forth in SEQ ID NO: 3.

22. The fusion protein of claim 1, wherein the cleavable linker comprises the amino acid sequence set forth in SEQ ID NO: 10.

23. The fusion protein of claim 1, wherein the CAR comprises the amino acid sequence set forth in SEQ ID NOs: 1-2 or 43-85.

24. A nucleic acid vector encoding the modified fusion protein of claim 1.

25. The vector of claim 24, wherein the vector is an expression vector.

26. A modified immune cell or precursor thereof, comprising the fusion protein of claim 1 or the nucleic acid vector of claim 24.

27. The modified immune cell of claim 26, wherein the immune cell is selected from the group consisting of a T cell, a macrophage, and a NK cell.

28. The modified immune cell of claim 26, wherein the immune cell is a T cell.

29. The modified immune cell of claim 28, wherein the T cell is selected from the group consisting of a CD4 T cell, a CD8 T cell, and a NK T cell.

30. The modified immune cell of claim 29, wherein the T cell is a CD8 T cell.

31. A method of treating cancer in a subject in need thereof, comprising administering to the subject an effective amount of a modified immune cell comprising a fusion protein comprising a chimeric antigen receptor linked to a second polypeptide by a linker domain;
wherein the CAR comprises an antigen binding domain, a transmembrane domain, and an intracellular domain, and wherein the second polypeptide prolongs the surface expression of the CAR, and wherein the linker is a cleavable linker.

32. The method of claim 31, wherein the second polypeptide is a Rab5 protein.

33. The method of claim 31, wherein the second polypeptide is a Rab11 protein.

34. The method of claim 31, wherein the linker is a self-cleaving peptide.

35. The fusion protein of claim 34, wherein the self-cleaving peptide is a 2A peptide.

36. The method of claim 31, wherein the CAR has a binding specificity for a cancer-related antigen.

37. The method of claim 36 wherein the cancer-related antigen is CD19.

38. The method of claim 36, wherein the cancer-related antigen is mesothelin.

39. The method of claim 36, wherein the cancer-related antigen is selected from a group consisting of: TSHR, CD19, CD123, CD22, CD30, CD171, CS-1, CLL-1, CD33, EGFRVIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

40. The method of claim 31, wherein the intracellular domain comprises a signaling domain selected from the group consisting of a 4-1BB signaling domain, a CD28 signaling domain, and a KIR2DS2 domain.

41. The method of claim 40, wherein the 4-1BB signaling domain comprises the amino acid sequence set forth in SEQ ID NO: 8.

42. The method of claim 40, wherein the KIR2DS2 domain comprises the amino acid sequence set forth in SEQ ID NO: 41.

43. The method of claim 31, wherein the intracellular domain comprises a CD3zeta signaling domain.

44. The method of claim 42, wherein the CD3zeta domain comprises an amino acid sequence set forth in SEQ ID NO: 9.

45. The method of claim 31, further comprising a CD8 leader sequence.

46. The method of claim 44, wherein the CD8 leader sequence comprises the amino acid sequence set forth in SEQ ID NO: 4.

47. The method of claim 31, wherein the transmembrane domain is a CD8 transmembrane domain.

48. The method of claim 46, wherein the CD8 transmembrane domain comprises the amino acid sequence set forth in SEQ ID NO: 5.

49. The method of claim 31, wherein the fusion protein comprises the amino acid sequence set forth in SEQ ID NO: 1.

50. The method of claim 31, wherein the fusion protein comprises the amino acid sequence set forth in SEQ ID NO: 2.

51. The method of claim 31, wherein the second polypeptide comprises the amino acid sequence set forth in SEQ ID NO: 3.

52. The method of claim 31, wherein the cleavable linker comprises the amino acid sequence set forth in SEQ ID NO: 10.

53. The method of claim 31, wherein the CAR comprises an amino acid sequence selected from the group consisting of SEQ ID NOs: 1-2 and 43-85.

54. The method of claim 31, wherein the cancer is selected from the group consisting of B cell acute lymphoblastic leukemia, chronic lymphocytic leukemia, myeloma, mesothelioma, ovarian cancer, lung cancer, squamous carcinoma, non-pulmonary adenocarcinoma, pancreatic cancer, stomach cancer, and colon cancer.

55. The method of claim 31, wherein the subject is human.


SEQUENCE LISTING (85 sequences)

SEQ ID NO: 1 - protein, 726 aa, synthetic construct
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGEGRGSLLTCGDVEENPGPRMAGRGGARRPNGPAAGNKICQFKLVLLGESAVGKSSLVLRFVKGQFHEYQESTIGAAFLTQTVCLDDTTVKFEIWDTAGQERYHSLAPMYYRGAQAAIVVYDITNTDTFARAKNWVKELQRQASPNIVIALAGNKADLASKRAVEFQEAQAYADDNSLLFMETSAKTAMNVNEIFMAIAKKLPKNEPQNATGAPGRNRGVDLQENNPASRSQCCSN

SEQ ID NO: 2 - protein, 726 aa, synthetic construct
MALPVTALLLPLALLLHAARPEQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGEGRGSLLTCGDVEENPGPRMAGRGGARRPNGPAAGNKICQFKLVLLGESAVGKSSLVLRFVKGQFHEYQESTIGAAFLTQTVCLDDTTVKFEIWDTAGQERYHSLAPMYYRGAQAAIVVYDITNTDTFARAKNWVKELQRQASPNIVIALAGNKADLASKRAVEFQEAQAYADDNSLLFMETSAKTAMNVNEIFMAIAKKLPKNEPQNATGAPGRNRGVDLQENNPASRSQCCSN

SEQ ID NO: 3 - protein, 216 aa, synthetic construct
MAGRGGARRPNGPAAGNKICQFKLVLLGESAVGKSSLVLRFVKGQFHEYQESTIGAAFLTQTVCLDDTTVKFEIWDTAGQERYHSLAPMYYRGAQAAIVVYDITNTDTFARAKNWVKELQRQASPNIVIALAGNKADLASKRAVEFQEAQAYADDNSLLFMETSAKTAMNVNEIFMAIAKKLPKNEPQNATGAPGRNRGVDLQENNPASRSQCCSN

SEQ ID NO: 4 - protein, 22 aa, synthetic construct
MALPVTALLLPLALLLHAARPE

SEQ ID NO: 5 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 6 - protein, 241 aa, synthetic construct
IVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 7 - protein, 241 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 8 - protein, 41 aa, synthetic construct
KRGRKKLLYIFKQPFMRPVQTTQEEDGCSCRFPEEEEGGCE

SEQ ID NO: 9 - protein, 113 aa, synthetic construct
LRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 24 aa, synthetic construct
SGGSGEGRGSLLTCGDVEENPGPR

SEQ ID NO: 11 - protein, 5 aa, synthetic construct
  REPEAT 1..5: note = repeat n times, where n is an integer of at least one
GSGGS

SEQ ID NO: 12 - protein, 5 aa, synthetic construct
  REPEAT 1..5: note = repeat n times, where n is an integer of at least one
GGGGS

SEQ ID NO: 13 - protein, 4 aa, synthetic construct
  REPEAT 1..4: note = repeat n times, where n is an integer of at least one
GGGS

SEQ ID NO: 14 - protein, 7 aa, synthetic construct
AAEPKSS

SEQ ID NO: 15 - protein, 17 aa, synthetic construct
AAEPKSSDKTHTCPPCP

SEQ ID NO: 16 - protein, 4 aa, synthetic construct
GGGG

SEQ ID NO: 17 - protein, 14 aa, synthetic construct
GGGGDKTHTCPPCP

SEQ ID NO: 18 - protein, 4 aa, synthetic construct
GGSG

SEQ ID NO: 19 - protein, 5 aa, synthetic construct
GGSGG

SEQ ID NO: 20 - protein, 5 aa, synthetic construct
GSGSG

SEQ ID NO: 21 - protein, 5 aa, synthetic construct
GSGGG

SEQ ID NO: 22 - protein, 5 aa, synthetic construct
GGGSG

SEQ ID NO: 23 - protein, 5 aa, synthetic construct
GSSSG

SEQ ID NO: 24 - protein, 5 aa, synthetic construct
DKTHT

SEQ ID NO: 25 - protein, 4 aa, synthetic construct
CPPC

SEQ ID NO: 26 - protein, 15 aa, synthetic construct
CPEPKSCDTPPPCPR

SEQ ID NO: 27 - protein, 12 aa, synthetic construct
ELKTPLGDTTHT

SEQ ID NO: 28 - protein, 10 aa, synthetic construct
KSCDKTHTCP

SEQ ID NO: 29 - protein, 7 aa, synthetic construct
KCCVDCP

SEQ ID NO: 30 - protein, 7 aa, synthetic construct
KYGPPCP

SEQ ID NO: 31 - protein, 15 aa, synthetic construct
EPKSCDKTHTCPPCP

SEQ ID NO: 32 - protein, 12 aa, synthetic construct
ERKCCVECPPCP

SEQ ID NO: 33 - protein, 17 aa, synthetic construct
ELKTPLGDTTHTCPRCP

SEQ ID NO: 34 - protein, 12 aa, synthetic construct
SPNMVPHAHHAQ

SEQ ID NO: 35 - protein, 216 aa, synthetic construct
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 36 - DNA, 369 bp, synthetic construct
  source 1..369: mol_type = other DNA
atggccttaccagtgaccgccttgctcctgccgctggccttgctgctccacgccgccaggccgggatccgctagcgagcagaaattaatctcagaggaggacctgcctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 37 - protein, 122 aa, synthetic construct
MALPVTALLLPLALLLHAARPGSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 38 - protein, 30 aa, synthetic construct
GAGCAGAAATTAATCTCAGAGGAGGACCTG

SEQ ID NO: 39 - protein, 11 aa, synthetic construct
SGGGGSGGGGS

SEQ ID NO: 40 - DNA, 222 bp, synthetic construct
  source 1..222: mol_type = other DNA
cccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgca

SEQ ID NO: 41 - protein, 74 aa, synthetic construct
PTEPSSKTGNPRHLHVLIGTSVVKIPFTILLFFLLHRWCSNKKNAAVMDQEPAGNRTVNSEDSDEQDHQEVSYA

SEQ ID NO: 42 - protein, 21 aa, synthetic construct
MALPVTALLLPLALLLHAARP

SEQ ID NO: 43 - protein, 372 aa, synthetic construct
MALPVTALLLPLALLLHAARPGSQAVLNQPASVSAALGQRVTISCNTNIGSPYDVQWYQQLPGKSPKTIIYGNSNRPSGVPVRFSGSKSGSTATLTIAGIQAEDEADYYCQSYDDSLDGRVVFGGGTQLTVLGGGSSRSSSSGGGGSGGGGEVQLVESGGDLVKPGGSLRLSCVASGFTFSNHEMYWVRQAPGKGLEWVARIYDSGSRTRYADAVKGRFTISRDNAKNTLYLQMNSLTAEDTAVYFCAGGNSRGWTEFGMDYWGPGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 44 - protein, 372 aa, synthetic construct
MALPVTALLLPLALLLHAARPGSSYVLTQSPSLSGSLGQKVTISCTGSRSNLGGNYVGWYQQVPGMAPRNVIYANDFRPSRIPARFSASKSGTSATLTISGLQAEDEADYFCSSWDDDLGSHVFGSGTQLTILGGGSSRSSSSGGGGSGGGGEEQLVEFGGDLVKPGGSLRLSCVASGFSFSSYDMSWVRQAPGKGLQWVASTNFDGSRTYYTDAVEGRFTISRDNARNTVYLQMNSLRAEDTAVYYCARGMGSWGASTFGYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 45 - protein, 369 aa, synthetic construct
MALPVTALLLPLALLLHAARPGSQSVLNQPPSVSGALGQTVTISCSGSHTNIGSGFDVQWYQQLPGKSPQTLVYANINRPSGVPARFSGSKSGSTATLTITGVQAEDEADYYCQSYDDNFDGHVFGSGTQLTVLGGGSSRSSSSGGGGSGGGGEVQLVQSGGDLVKPGGSLRLSCVASGFTFSNYTMSWVRQAPGQGLQWVAYINYDGSITYYAEAVKGRFTVSRDNAKNTLYLQMNSLRAEDTAVYFCVKETFMIPTEWGQGTLITVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 46 - protein, 370 aa, synthetic construct
MALPVTALLLPLALLLHAARPGSQSVLNQPPSVSGALGQTVTISCSGSHTNIGSGFDVQWYQQLPGKSPQTIIYANINRPSGVPARFSGSKSGSTATLTITGVQAEDEADYYCQSYDDNFDYVFGSGTQLTVLGGGSSRSSSSGGGGSGGGGEEQLVEFGGDLVKPGGSLRLSCVVSGFDFSRYGMSWVRQSPGKGLQWVADISYRGTTFYADDVKGRFTVSRDSAKNTLYLEMTSLTVEDTAVYYCANSANWYVSPFDHWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 47 - protein, 368 aa, synthetic construct
MALPVTALLLPLALLLHAARPGSQPVLTQPPSVSGSLGQRVTISCSGSTNTIGVVGANWYQQLPGKSPKLLVYSDGVRPSGVPDRFSGSRSANSDTLTITGLQAEDEADYYCQSFDSTHNAIVFGGGTHLTVLGGGSSRSSSSGGGGSGGGGEVQLVETGGDLVKPAGSLRLSCAASGFPFSGYSMTWVRQAPEKGLQLVAGINSDGSHTYYTDDVKGRFTISRDNTKYILYLQMNSLRAEDTAMYYCGSHSWGNFHYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 48 - protein, 371 aa, synthetic construct
MALPVTALLLPLALLLHAARPGSQPVLTQPPSVSGSLGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVQSGGDLVKPGGSLRLSCVASGFTFSDNYMSWIRQVPGKGLQWVSRISTDGRGTSYADAVKGRFTISRDNAKNTLYLQMNSLRDEDTAVYYCARRSGIWGFDYWGQGALVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 49 - protein, 373 aa, synthetic construct
MALPVTALLLPLALLLHAARPGSSYELTQPPSMSAALGQRVTISCTGSNTNIGSGYDVQWYQQVPGKSPTPLIYGNTNRPSGVPVRFSGSKSGNTATLTITGIQAEDEADYYCQSYDDNFDGHHVFGSGTQLTVLGGGSSRSSSSGGGGSGGGGEVQLVESGGDLVKPAGSLRLSCVASGFTFSNYGMNWVRQAPGKGLQWVAGINSGGSTTTYADVVKGRFTISRDNAKNTLYLQMNSLGAEDTAVYYCARVDYSSSWLVDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 50 - protein, 369 aa, synthetic construct
MALPVTALLLPLALLLHAARPGSQSVLTQPASVSGSLGQKVTISCTGSRSNIGANYVGWYQQVPGIGPRTVIFGYNYRPSGVPDRFSSSKSGNSATLTISGLQAEDEAEYYCSSWDDSLRGTVFGSGTQLTVLGGGSSRSSSSGDGGSGGGGEVQLVESGGDLVKPGESLRLSCVASGFTFSSYGMSWVRQSPGKGLQWVADISYGGGTYYADAVKGRFTISRDNARNTVYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 51 - protein, 372 aa, synthetic construct
MALPVTALLLPLALLLHAARPGSQPVLTQPPSVSAALGQRVTLSCTGSKTNIGSGYDVQWYQQFPGKSPKTIIYGNSNRFSGVPARFSGSKSGSIATLTITGIQAEDEADYYCQSFDDNVDGYVFGSGTQLTILGGGSSRSSSSGGGGSGGGGEVQLVESGGDLVKPGGSLRLSCVASGFTFSSDSIHWVRQAPGKGLQWVAYINSGGSTTYYADAVKGRFTISRDNARNTVYLKMNSLRAEDTAVYYCAKDSGFGSWYLESWGQGTLVTVSPASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 52 - protein, 369 aa, synthetic construct
MALPVTALLLPLALLLHAARPGSQAVLNQPASVSGALGQTVTISCSGSHTNIGSGFDVQWYQQLPGKSPQTIIYANINRPSGVPARFSGSKSGSTATLTITGVQAEDEADYHCQSFDDDLDGPVFGGGTHLTVLGGGSSRSSSSGGGGSGGGGEVQLVQSGGDLVKPGGSLKISCVASGLTFNRNYMTWIRQAPGKGLQWVSEINPDGSTTSYTDAVRGRFTISRDNAKNTLYLQMNSMRDEDTAVYYCARSSWGSFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 53 - protein, 365 aa, synthetic construct
MALPVTALLLPLALLLHAARPGSQPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGGGGSGGGGEVQLVESGGDLVKPGGSLRLSCVASGFTFSDYSMSWVRQSPGKGLQWVAAIMTSGSTYYADAVKGRFTISRDNAKNTLYLQMDSLRAEDTAVYYCAHDYWFDFWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 54 - protein, 369 aa, synthetic construct
MALPVTALLLPLALLLHAARPGSSYELTQPPSATVTLRQTARLTCGGENIGRKSVQWYQQKPGQSPLLIIYADSSRPSGIPERFSGANSGNTASLTISGALAEDEADYYCQIWDRNINVFGSGTQLTVLGGGSSRSSSSGGGGSGGGGEEQLVEFGGDLVKPAGSLRLSCVASGFTFSSYDMSWVRQAPGKGLQWVATISYDGSSTYYTDAVKGRFTVSRDNAKNTLYLQMNSLRAEDTALYYCAKDSGSWSHFYGLDTWGHGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 55 - protein, 368 aa, synthetic construct
MALPVTALLLPLALLLHAARPGSQPVLTQPPSVSGALGQTVTISCSGSHTNIGSGFDVQWYQQLPGKSPQTLVYANINRPSGVPARFSGSKSGSTATLTITGVQAEDEADYYCQSYDDNFDYVFGSGTQLTVLGGGSSRSSSSGDGGSGGGGEVQLVESGGGLMKPGGSLKLSCLASGFTFSRYYMYWVRQAPGKGLQWVAQISSDGRRTYYADAVQGRFTISRDNVKNTVYLQMNSLSDEDTAVYYCVREDYFDFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 56 - protein, 370 aa, synthetic construct
MALPVTALLLPLALLLHAARPGSQSVLNQPPSVSAALGQRITISCTGSHTNIGSGYDVQWYQQLPGKSPRTIIYGNSDRPSGVPVRFSGSKSGNTATLTITGIQAEDEADYYCQSYDDNLNDPYVFGSGTQLTVLGGGSSRSSSSGGGGSGGGGEVQLVQSGGDLVKPGGSLRLSCVASGFAFSGKTMQWVRQAPGKGLQWVAYIQHDGSRTSYADAVKGRFTISRDNAKNTLYLQMDSLRAEDTAVYYCADHPWYGPEYWGQGTPVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 57 - protein, 374 aa, synthetic construct
MALPVTALLLPLALLLHAARPGSSYELTQPPSVSAALGQGVTISCIGSDTNIGNHYDVQWYQQLPGKSPRTIIYGNSDRPSGVPGRFSGSKSGNTATLTITGIQAEDEADYHCQSYDDDLDGHIVFGGGTHLTVLGGGSSRSSSSGGGGSGGGGEVQLVQSGGDLVKPGGSLRLSCVASGFTFNKYHMSWVRQAPGKGLQWVGKITSDGSATGYADAVKGRFTISRDNAKNTFYLQMDSLRAEDTAMYYCARDQSSWGTGNFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 58 - protein, 369 aa, synthetic construct
MALPVTALLLPLALLLHAARPGSQSVLTQPTSVSGSLGQRVTISCTGSSSNVGYGNYVGWYQQLPGTGPRTLIYGSHYRPSGVPDRFSGSSSGSSATLTISGLQAEDEADYYCSSYDSTLSGYVFGSGTQLTILGGGSSRSSSSGGGGSGGGGEVQLVESGGDLVKPGGSLRLSCVGSGFTLSSYWMSWVRQAPGKGLQWIAEISGSGNTIYYGDAVKGRFTISRDNAKNTLYLQMNSLRDEDTAVYYCATEYYWNIDNWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 59 - protein, 371 aa, synthetic construct
MALPVTALLLPLALLLHAARPGSQPVLTQPPSVSAALGQRVTISCTGSKTNIGSGYDVQWYQQLPGKSPKTIIYGNSNRPSGVPARFSGSKSGSIATLTITGIQAEDEADYYCQSFDDNVDGYVFGSGTQLTILGGGSSRSSSSGGGGSGGGGEVPLVESGGDLVKPGGSLRLSCVASGFTFSSYGMSWVRQAPGKGLQWVAYINGGGSSTSYADAVRGRFTISRDNAENTLYLQMNSLRADDTAVYYCSNYYGIYWDSDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 60 - protein, 369 aa, synthetic construct
MALPVTALLLPLALLLHAARPGSQSALTQTASVSGSLGQKVTISCTGSRSNIGANYVGWYQQVPGIGPRTVIFGYNYRPSGVPDRFSSSKSGNSATLTISGLQAEDEAEYYCSTWDSSLNAYVFGSGTQLTVLGGGSSRSSSSGGGGSGGGGEVPLVESGGDLVKPGGSLRLSCVASGFTLSNYGMSWVRQSPGKGLQWVAAIRSGGNTYYADAVKGRFTISRDNAKNTLYLQMNSLRAEDTAVYYCASQWGDWGHFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 61 - protein, 368 aa, synthetic construct
MALPVTALLLPLALLLHAARPGSQAVLTQPASVSGPLGQRVTISCSGSTNNIGIVGAAWYQQFPGKAPKLLVYSDGSRPSGVPDRFSGSNSGNSATLTISGLQAEDEADYYCQSVDATHGTILFGGGTQLTVLGGGSSRSSSSGGGGSGGGGEVQLVETGGDLVKPAGSLRLSCAASGFPFSGYSMTWVRQAPEKGLQLVAGINSDGSHTYYTDDVKGRFTVSRDNTKYILYLQMNSLRVEDTAIYYCGSHSWGNFHYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 62 - protein, 364 aa, synthetic construct
MALPVTALLLPLALLLHAARPGSQSVLNQPPSVSVSLGQTATISCSGESLSKYYAQWFQQKAGQVPVLVIYKDTERPSGIPDRFSGSSSGNTHTLTISGARAEDEADYYCESEVSPDTIVFGGGTHLTVLGGGSSRSSSSGGGGSGGGGEVQLVESGGDLVKPGGSLRLSCVASGFTFSSYAMSWVRQAPGEGLQWVAYINSAGSTTYYADAVKGRFTISRDNARNTVYLQMNSLRAEDTAVYYCGRYYYGLDYWGPGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 63 - protein, 366 aa, synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTISGARAEDEADYYCESAVSSDTAVFGSGTQLTVLGGGSSRSSSSGGGGSGGGGEVQLVESGGDMVKPGGSLRLSCVASGFTFSSFWMYWVRQAPGKGLQWVGGINSGGSITSYADAVKGRFTISRDNARNTLYLQMDSLRDEDTAVYYCAKPDIYGSFDHWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 64 - protein, 369 aa, synthetic construct
MALPVTALLLPLALLLHAARPGSQPVLTQPPSVSGSLGQRVTISCTGSSSNVGYGDYVGWYQQLPGTSPRTLIYSSSSRPSGVPDRFSGSRSGSTATLTISGLQAEDEADYHCSSYDTSLNGFVFGSGTQLTILGGGSSRSSSSGGGGSGGGGELTLQESGPGLVKPAQTLSLTCTVSGDSVTSSHFWHWIRQRPGRGLEWMGYWSDITIYSPTFQGRISITVDTARNQFSLHLNSVTTEDSAVYFCARYNGYVDYLDVWGQGTLITVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 65 - protein, 367 aa, synthetic construct
MALPVTALLLPLALLLHAARPGSDIVMTQTPLSLSVSPGEPASISCKASQSLLHSNGNTYLYWFRQKPGQSPQRLIYLVSNRDAGVPDRFSGSGSGTDFTLRISRVEADDSGIYYCGQGKQDPITFGEGTHLEIKGGSSRSSSSGGGGSGGGGELTLQESGPGLVKPSQTLSLTCVVSGGSVTTSDYWNWLRQRPGRGLEYMGYWKGRTEYNPAFQGRISITADTAKNQFSLQLNSVTADDTAIYYCAHWGSGNLDYWGPGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 66 - protein, 369 aa, synthetic construct
MALPVTALLLPLALLLHAARPGSQSVLNQPPSVSGALGQTVTISCSGSHTNIGSGFDVQWYQQLPGKSPQTLVYANINRPSGVPVRFSGSKSGTTATLTITGIQAEDEADYYCQSYDDNVDGYVFGSGTQLTVLGGGSSRSSSSGGGGSGGGGEVQLVQSGGXLVKPGGSLRLSCVASGFTLSSNYMDWVRQAPGKGPQWVAQISTDGSRTYYADAVKGRFTISRDTAKNTLYLQMNSLRAEDTAMYYCAKNEYLAPTYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 67 - protein, 372 aa, synthetic construct
MALPVTALLLPLALLLHAARPGSDIVMTQTPLSLSVSPGETASISCKASQSLLHSNGNTYLNWFRQKPGQSPQVLIYQVSNRDTGVPDRFSGSGSGTDFTLRISRVEADDAGVYYCGQGIQYPPTFGAGTKVELKGGSSRSSSSGGGGSGGGGEEQLVEFGGDLVKPGGSLRLSCVASGFTFSSLSMNWIRQAPGKGLQWVSQISPDGTITGYTDAVKGRFTISRDNAKNTLYLQMNSLRDEDTAIYYCAKSRYTDTYPFDYWGPGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 68 - protein, 363 aa, synthetic construct
MALPVTALLLPLALLLHAARPGSSSVLTQPPSVSVSLGQTATISCSGESLSKYYTQWFQQKAGQAPILVIYKDTERPSGIPDRFSGSSSGNTHTLTISGARAEDEADYYCESLVDSDAYMFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 69 - protein, 364 aa, synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTITGIQAEDEGDYHCHSYDENLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 70 - protein, 363 aa, synthetic construct
MALPVTALLLPLALLLHAARPGSQSALTQPSSVSVSLGQTATISCSGESLNKYYVQWFQQKAGQVPILVIYRDTERPSGIPDRFSGSSSGNTHTLTITGTRAEDEADYYCESLVNDNTYVFGSGTQLTVLGGGSSRSSSSGGGGSGGGGEVQLVETGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 71 - protein, 363 aa, synthetic construct
MALPVTALLLPLALLLHAARPGSQSVLNQPPSVSVSLGQTATISCSGESLNKYYTQWFQQKAGQAPVLVIYKDTERPSGIPDRFSGSSSGNTHTLTISGARAEDEADYYCESVDSTDTYVFGSGTQLTILGGGSSRSSSSGGGGSGGGGEVHLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 72 - protein, 364 aa, synthetic construct
MALPVTALLLPLALLLHAARPGSQSVLTQPASVSVSLGQTATISCSGESLSKYYTQWFQQKAGQAPVLVIYKDTERPSGIPDRFSGSSSGNTHTLTISGAQAEDEADYYCQSYDDNVDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 73 - protein, 363 aa, synthetic construct
MALPVTALLLPLALLLHAARPGSQSALTQPSSVSVSLGQTATISCSGESLTKYYVQWFQQKAGQAPVLVIYRDTERPSGIPDRFSGSSSGNTHTLTISGARAEDEADYYCESMDGREAYVFGSGTQLTVLGGGSSRSSSSGGGGSGGGGEVQLVESGGDLVKPGGSLRLSCVASGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 74 - protein, 364 aa, synthetic construct
MALPVTALLLPLALLLHAARPGSQSVLTQPASVSVSLGQTATISCSGESLSKYYAQWFQQKAGQAPVLVIYKDTERPSGIPDRFSGSSSGNTHTLTITGIQAADEADYYCQSYDDNLDGYVFGSGTQLTVLGGGSSRSSSSGGGGSGGGGEVQLVESGGDLVKPGGSLRLSCVGSGFTFSSYWMSWVRQAPGKGLQWIADISDSGSSTDYADAVKGRFTISRDNAKNTLYLQMNSLRAEDTAMYYCARGLGMDYWGHGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 75 - protein, 365 aa, synthetic construct
MALPVTALLLPLALLLHAARPGSDIVMTQTPLSLSVSPGEPASISCKASQSLLHSDGNTYLHWYLQKPGQSPQLLIYSVSNRFTGVSDRFSGSGSGTDFTLRISRVEADDTGVYYCGQGTHLPFTFGQGTKLEIKGGSSRSSSSGGGGSGGGGEVQLVESGGDLVKPGGSLRLSCAASGFTFSHYDMNWVRQAPGKGLQWVASIAYDGSNTYYTDAVKGRFTISRDNAKNILFLLVNSLRAEDTAIYYCARGLVYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 76 - protein, 371 aa, synthetic construct
MALPVTALLLPLALLLHAARPGSSYELTQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTILGGGSSRSSSSGGGGSGGGGEVQLVESGGDLVKPGGSLRLSCVASGLTFSDNYMSWIRQVPGKGLQWVSRISTDGRGTSYADAVKGRFTISRDNAKNTLYLQMNSLRDEDTAVYYCARRSGIWGFDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 77 - protein, 363 aa, synthetic construct
MALPVTALLLPLALLLHAARPGSSSVLTQPPSVSVSLGQTATISCSGESLSEYYAQWFQQKAGQAPVLVIYKDTERASGIPDRFSGSSSGNTHTLTISGARAEDEADYYCESAVNSDGIVFGGGTHLTVLGGGSSRSSSSGGGGSGGGGEVQLVESGGDLVKPAGSLRLSCVTSGFTFSSYAMSWVRQAPGKGLQWVGSINSGGSTTSYADAVKGRFTISRDNAKNTVYLQMNSLRAEDTAVYYCARWWDFEYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 78 - protein, 368 aa, synthetic construct
MALPVTALLLPLALLLHAARPGSQSVLTQPTSVSGSLGQRVTISCSGSTNNIGIVGASWYQQLPGKAPKLLVYSDGDRPSGVPDRFSGSNSGNSDTLTITGLQAEDEADYYCQSFDTTLDAYVFGSGTQLTVLGGGSSRSSSSGGGGSGGGGEEQLVEFGGGLVRPGGSLRLSCVASGFTFSSYGMTWVRQSPGKGLQWVATISDTGSTYYADTVKGRFSISRDNAKNTLYLQMNSLRADDTALYYCTKDLGVWGMDNWGPGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 79 - protein, 362 aa, synthetic construct
MALPVTALLLPLALLLHAARPGSSSVLTQPPSVSVSLGQTATISCSGESLNDYYAQWFQQKAGQVPVLVIYRDTERPSGIPDRFSGSSSGNTHTLTISGARAEDEADYYCESEVSAGGAVFGEGTHLTVLGGGSSRSSSSGGGGSGGGGEVQLVETGGDLVKPGGSLRLSCVASGFTLRDYSMTWVRQTPEKGLQLVAGISTGGSTTYYIDAVKGRFTISRDNAKNTVYLQMNSLRAEDTAMYHCTTTALGYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 80 - protein, 363 aa, synthetic construct
MALPVTALLLPLALLLHAARPGSSSVLTQPPSVSVSLGQTATISCSGESLSEYYAQWFQQKAGQAPVLVIYKDTERPSGIPDRFSGSSSGNTHTLTISGARAEDEADYYCASADSSAIPVFGGGTHLTVLGGGSSRSSSSGGGGSGGGGEVQLVESGGDLVKPGGSLRLSCVASGFNFGNYAVSWVRQAPGKGLQWVAGISNDGTITGYTDVVEGRFIISRDNAKNTLYLQMNSLRAEDMAIYYCVAGGFLDYWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 81 - protein, 371 aa, synthetic construct
MALPVTALLLPLALLLHAARPGSQSVLTQPPSVSGSPGQRVTISCSGTTKNIGIVGASWYQQLPGKAPKLLVHSDGFRPSGVPDRFSASNSGDSDILTITGLQAEDEADYYCQSFDTTLDAHVFGSGTQLTVLGGGSSRSSSSGGGGSGGGGEVPLVESGGDLVKPGGSLRLSCVASGFTFSSYGMSWVRQSPGKGLQWVAGITYDGSNTYYTDTVRGRFTISRDNARNTVYLQMNSLRAEDTAVYYCVASPLYGTYGFDYWGHGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 82 - protein, 368 aa, synthetic construct
MALPVTALLLPLALLLHAARPGSQSVLTQPPSVSGSLGQKVTISCTGSSSSVGTHYVGWYQIPPGIGPRTVIYNNDYRPSGVPDRFSGSKSGNSATLTISGLQAEDEADYYCSSWDDSLGAYVLGSGTQLTILGGGSSRSSSSGGGGSGGGGELTLQESGPGLVKPSQTLSLTCAVSGGSVIRGYYWTWIRQRPGRGLEWMGYWAGSTDYNPAFQGRIFITAGTAKNQFSLEMTSMTTDDTAIYYCVGSLRGGWLFDNWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 83 - protein, 363 aa, synthetic construct
MALPVTALLLPLALLLHAARPGSQPVLTQPPSVSVSLGQTATISCSGESLSKYYAQWFQQKAGQAPVLVIYKDTERPSGIPDRFSGSSSGNTHTLTISGARAEDEADYYCESAVSTETVVFGGGTHLTVLGGGSSRSSSSGGGGSGGGGEVQLVETGGDLVMPAGSLRLSCVASGFTFSSSVMSWVRQAPGKGLEWVAGINGGGSFAAYGDAVKGRFTISRDNAKNTLYLQMNSLRAEDTAMYYCVGGGYMDFWGPGTSLFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 84 - protein, 366 aa, synthetic construct
MALPVTALLLPLALLLHAARPGSQSVLTQPTSVSGSLGQRVTISCSGSTNDIGIIGAAWYQQLPGKAPKLLLYSNKNRPSGVPDRFSGSNSGISATLTITGLQAEDEADYYCQSVDPALHTYVFGSGTQLTILGGGSSRSSSSGGGGSGGGGEVQLVEIGGDLVKPEGSLRLSCVVSGFTFSGYDMNWVRQAPGKGLQWVAYISSDGRRTVYTDAVKGRFTISRDNAKNTLYLQMNSLKSEDTAVYYCVKGGWLDFWGQGTLVTVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 85 - protein, 366 aa, synthetic construct
MALPVTALLLPLALLLHAARPGSDIVMTQNPLSLAVTPGELATISCRASQSLLHSDGKSYLNWYLQKPGQTPRPLIYEASRRFSGVSDRFNGSGSGTDFTLKISRVEAEDVGVYYCQQGLHFPPTFGAGTKVELKGGSSRSSSSGGGGSGGGGEVQLVESGGDLVKPGGSLRLSCAASGFTFTDYDMSWVRQAPGKGLQWVAAISYDGSSTYYTDAVKGRFTISRDNARNTLYLQMSSLRAEDTAMYYCVVSNMNLWGHGTSVFVSSASEQKLISEEDLPSGGGGSGGGGSSPTEPSSKTGNPRHLHVLIGTSVVKIPFTILLFFLLHRWCSNKKNAAVMDQEPAGNRTVNSEDSDEQDHQEVSYA